SEQUENCE LISTING (8 sequences)

SEQ ID NO: 1 - protein, 6 aa, Homo sapiens
HGSHDN

SEQ ID NO: 2 - protein, 12 aa, Homo sapiens
QSYDRYTHPALL

SEQ ID NO: 3 - protein, 17 aa, Homo sapiens
FIRYDGSNKYYADSVKG

SEQ ID NO: 4 - protein, 7 aa, Homo sapiens
YNDQRPS

SEQ ID NO: 5 - protein, 9 aa, Homo sapiens
FTFSSYGMH

SEQ ID NO: 6 - protein, 13 aa, Homo sapiens
SGSRSNIGSNTVK

SEQ ID NO: 7 - protein, 115 aa, Homo sapiens [sequence omitted]

SEQ ID NO: 8 - protein, 112 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

(19) United States
(12) Patent Application Publication
(10) Pub. No.: US 2010/0172862 A1
(43) Pub. Date: Jul. 8, 2010

(54) STABLE ANTIBODY COMPOSITIONS AND METHODS OF STABILIZING SAME

(75) Inventors: Ivan R. Correia, Winchester, MA (US); Czeslaw H. Radziejewski, Westborough, MA (US); Wolfgang Fraunhofer, Shrewsbury, MA (US); Nicholas W. Warne, Andover, MA (US); Angela Kantor, Pepperell, MA (US)

Correspondence Address: McCarter & English, LLP / Abbott Laboratories Ltd., 265 Franklin Street, Boston, MA, 02110, US

(73) Assignee: ABBOTT LABORATORIES, Abbott Park, IL (US)

(21) Appl. No.: 12/625,057

(22) Filed: Nov. 24, 2009

Related U.S. Application Data
(60) Provisional application No. 61/118,528, filed on Nov. 28, 2008.

(51) Int. Cl. A61K 38/20; C07K 16/18; A61K 39/395; A61K 38/19; A61K 38/21; A61P 37/04

(52) U.S. Cl. 424/85.2; 530/387.1; 424/130.1; 424/141.1; 424/85.1; 424/85.6

(57) ABSTRACT

The invention provides compositions and methods for inhibiting fractionation of immunoglobulins comprising a lambda light chain based on the observation that iron, in the presence of histidine, results in increased fragmentation of a recombinant fully human IgG molecule containing a lambda light chain due to cleavage in the hinge region. The invention further provides an aqueous pharmaceutical formulation comprising an antibody, or antigen-binding portion thereof, that binds the p40 subunit of IL-12/IL-23 and a buffer system comprising histidine, wherein the formulation has enhanced stability, including enhanced resistance to fragmentation.

### CROSS-REFERENCE TO RELATED APPLICATION

This application claims priority to U.S. Provisional Application Ser. No. 61/118,528 filed on Nov. 28, 2008, the contents of which are incorporated herein.

### BACKGROUND OF THE INVENTION

Interleukin-12 (IL-12) and the related cytokine IL-23 are members of the IL-12 superfamily of cytokines that share a common p40 subunit (Anderson et al. (2006) Springer Semin. Immunopathol. 27:425-42). IL-12 primarily stimulates differentiation of Th1 cells and subsequent secretion of interferon-gamma, whereas IL-23 preferentially stimulates differentiation of naïve T cells into effector T helper cells (Th17) that secrete IL-17, a proinflammatory mediator (Rosmarin and Strober (2005) J. Drugs Dermatol. 4:318-25; Harrington, et al. (2005) Nature Immunol. 6:1123-32; Park et al. (2005) Nature Immunol. 6:1132-41).

Human interleukin 12 (IL-12) is a cytokine with a unique structure and pleiotropic effects (Kobayashi, et al. (1989) J. Exp. Med. 170: 827-845; Seder, et al. (1993) Proc. Natl. Acad. Sci. 90:10188-92; Ling, et al. (1995) J. Exp. Med. 154:116-127; Podlaski, et al. (1992) Arch. Biochem. Biophys. 294:230-237). IL-12 is a heterodimeric protein comprising a 35 kDa subunit (p35) and a 40 kDa subunit (p40) which are both linked together by a disulfide bridge (referred to as the “p70 subunit”). The heterodimeric protein is produced primarily by antigen-presenting cells such as monocytes, macrophages and dendritic cells. These cell types also secrete an excess of the p40 subunit relative to the p70 subunit. The p40 and p35 subunits are genetically unrelated and neither has been reported to possess biological activity, although the p40 homodimer may function as an IL-12 antagonist. IL-12 plays a critical role in the pathology associated with several diseases involving immune and inflammatory responses. A review of IL-12, its biological activities, and its role in disease can be found in Gately et al. (1998) Ann. Rev. Immunol. 16: 495-521.

Functionally, IL-12 plays a central role in regulating the balance between antigen specific T helper type (Th1) and type 2 (Th2) lymphocytes, which govern the initiation and progression of autoimmune disorders, and is critical in the regulation of Th₁ lymphocyte differentiation and maturation. Cytokines released by the Th1 cells are inflammatory and include interferon γ (IFN γ, IL-2 and lymphotoxin (LT). Th2 cells secrete IL-4, IL-5, IL-6, IL-10 and IL-13 to facilitate humoral immunity, allergic reactions, and immunosuppression.

Human interleukin 23 (IL-23) is a heterodimeric protein comprising a 19 kDa subunit (p19) and the common 40 kDa subunit (p40), which are linked together by a disulfide bridge. IL-23, similarly to IL-12, is produced primarily by antigen-presenting cells such as monocytes, macrophages and dendritic cells. The dominant role of IL-23 involves the stimulation of a subset of CD4+ T-cells (also referred to as IL-17 T cells or Th17) to produce the cytokine IL-17. IL-17, in turn, is a critical component in the establishment and perpetuation of autoimmune inflammation, inducing the production of proinflammatory cytokines by endothelial cells and macrophages (Kastelein et al. (2007) Annu. Rev. Immunol. 25:221-42).

Consistent with the preponderance of Th1 responses in autoimmune diseases and the proinflammatory activities of IFN γ and IL-17, IL-12 and IL-23 play a major role in the pathology associated with many autoimmune and inflammatory diseases such as rheumatoid arthritis (RA), multiple sclerosis (MS), Psoriasis, Insulin-Dependent Diabetes Mellitus, and Crohn's disease (CD), for example.

Elevated levels of IL-12 p70 have been detected in the synovia of RA patients compared with healthy controls (Morita et al. (1998) Arthritis and Rheumatism 41:306-314). Cytokine messenger ribonucleic acid (mRNA) expression profile in the RA synovia identified predominantly Th1 cytokines. (Bucht et al. (1996) Clin. Exp. Immunol. 103:347-367). Using gene-targeted mice lacking the p19 subunit of IL-23 or the p40 subunit of IL-12/23, IL-23 was shown to be critical for the development of collagen induced arthritis (Murphy et al. (2003) J. Exp. Med. 198(12):1951-1957).

Human patients with MS have demonstrated an increase in IL-12/IL-23 expression as documented by p40 mRNA levels in acute MS plaques. (see, e.g., Windhagen et al. (1995) J. Exp. Med. 182:1985-96). In addition, ex vivo stimulation of antigen-presenting cells with CD40L-expressing T cells from MS patients resulted in increased IL-12 production compared with control T cells, consistent with the observation that CD40/CD40L interactions are potent inducers of IL-12. Using gene-targeted mice lacking IL-23, IL-23 was shown to be critical for autoimmune inflammation of the brain (Cua et al. (2003) Nature 421:7440748).

Increased expression of IFN γ and IL-12 has been observed in the intestinal mucosa of patients with CD (Fail et al. (1994) J. Interferon Res. 14:235-238; Parronchi et al. (1997) Am. J. Path. 150:823-832; Monteleone et al. (1997) Gastroenterology 112:1169-1178, and Berrebi et al. (1998) Am. J. Path. 152:667-672). The cytokine secretion profile of T cells from the lamina propria of CD patients is characteristic of a predominantly Th1 response, including greatly elevated IFN γ levels (Fuss, et al. (1996) J. Immunol. 157:1261-1270). Moreover, colon tissue sections from CD patients show an abundance of IL-12 expressing macrophages and IFN y expressing T cells (Parronchi et al. (1997) Am. J. Path. 150:823-832). Increased expression of IL-23 has also been observed in patients with Crohn's disease and in mouse models of inflammatory bowel disease. IL-23 is essential for T cell-mediated colitis and to promote inflammation through IL-17- and IL-6-dependent mechanisms in mouse models of colitis (see e.g., review by Zhang et al., (2007) Intern. Immunopharmacology 7:409-416).

The overexpression of IL-12/IL-23 p40 and IL-23 p19 messenger RNA in psoriatic skin lesions suggests that the inhibition of IL-12 and IL-23 with a neutralizing antibody to the IL-12/23 p40 subunit protein may offer an effective therapeutic approach for the treatment of psoriasis (Yawalkar, et al. (1998) J. Invest. Dermatol. 111: 1053-57; Lee et al. (2004) J. Exp. Med. 199: 125-30; Shaker et al. (2006) Clin. Biochem. 39: 119-25; Piskin et al. (2006) J. Immunol. 176: 1908-15; see also recent reviews by Torti et al. (2007) J. Am. Acad. Dermatol. 57(6):1059-1068; Fitch et al. (2007) Current Rheumatology Reports 9:461-467).). Both cytokines contribute to the development of the type IT helper cell (Th1) immune response in psoriasis, but each has a unique role (Rosmarin and Strober (2005) J. Drugs Dermatol. 4:318-25; Hong et al. (1999) J. Immunol. 162:7480-91; Yawalkar, et al. (1998) J. Invest. Dermatol. 111:1053-57). Such therapeutic approaches for the treatment of psoriasis are clearly needed in the art.

Due to the roles of human IL-12 and IL-23 in a variety of human disorders, therapeutic strategies have been designed to inhibit or counteract IL-12/IL-23 activity. In particular, antibodies that bind to, and neutralize, the p40 subunit of IL-12/IL-23 have been sought as a means to inhibit IL-12/IL-23 activity. Some of the earliest antibodies were murine monoclonal antibodies (mAbs), secreted by hybridomas prepared from lymphocytes of mice immunized with IL-12 (see e.g., PCT Publication No. WO 97/15327 to Strober et al.; Neurath et al. (1995) J. Exp. Med. 182:1281-1290; Duchmann et al. (1996) J. Immunol. 26:934-938). These murine IL-12 antibodies are limited for their use in vivo due to problems associated with administration of mouse antibodies to humans, such as short serum half life, an inability to trigger certain human effector functions and elicitation of an unwanted immune response against the mouse antibody in a human (the “human anti-mouse antibody” (HAMA) reaction).

In general, attempts to overcome the problems associated with use of fully-murine antibodies in humans, have involved genetically engineering the antibodies to be more “human-like.” For example, chimeric antibodies, in which the variable regions of the antibody chains are murine-derived and the constant regions of the antibody chains are human-derived, have been prepared (Junghans et al. (1990) Cancer Res. 50:1495-1502; Brown et al. (1991) Proc. Natl. Acad. Sci. USA 88:2663-2667; Kettleborough et al. (1991) Protein Engineering 4:773-783). However, because these chimeric and humanized antibodies still retain some murine sequences, they still may elicit an unwanted immune reaction, the human anti-chimeric antibody (HACA) reaction, especially when administered for prolonged periods.

A preferred IL-12/IL-23-inhibitory agent to murine antibodies or derivatives thereof (e.g., chimeric or humanized antibodies) is an entirely human anti-IL-12/IL-23 antibody, since such an agent should not elicit the HAMA reaction, even if used for prolonged periods. Recombinant human antibodies that bind the p40 subunit of human IL-12/IL-23 with high affinity and slow dissociation kinetics and that have the capacity to neutralize human IL-12, including hIL-12-induced phytohemagglutinin blast proliferation and hIL-12-induced human IFNγ production, have been described (see U.S. Pat. No. 6,914,128).

The selectivity of monoclonal antibodies (Mabs) for specific antigens makes them excellent therapeutic candidates. However, due to the structure of antibody molecules they are vulnerable to enzymatic and non-enzymatic degradation. For example, storage of antibodies at elevated temperatures for extended periods of time results in a non-enzymatic degradation of the antibody (Connell, G. E. and R. H. Painter (1966) Can. J. Biochem. 44(3):371-9; Cordoba, A. J. et al. (2005) J. Chromatogr. B Analyt. Technol. Biomed. Life Sci. 818(2):115-21; Cohen, S. L. et al. (2007) J. Am. Chem. Soc. 129(22):6976-7).

Human immunoglobulin gamma (IgG) antibodies are generally composed of two identical light chains and heavy chains. The heavy chain is of the gamma type whereas the light chain can either be of the kappa or lambda type, differing in their carboxyl terminal constant regions. Inter-chain disulfide bridges hold the heavy chains together. The number of disulfide bridges varies among the IgG subclasses. For IgG1, for example, there are two inter-heavy chain disulfide bridges and one disulfide-bridge holding each light and heavy chain together.

An IgG molecule is composed of an Fc region and two Fab regions that are linked by a hinge region. The hinge region is divided into 3 portions—the upper, the core and the lower regions (FIG. 1). The upper region links the Fab arms to the core whereas the lower region links the Fc portion to the core. The core region contains the inter-chain disulfide bonds and has high proline content. The length of the hinge region varies among the IgG subclasses and provides flexibility to the Fab arms, allowing both variation of the angle between the arms as well as freedom of rotation around their axis. As a result of its flexibility, the hinge region is exposed and thus is easily perturbed by temperature and storage for prolonged periods of time. For example, the hinge region is accessible to proteases such as papain and lys-C, which are routinely used to generate Fc and Fab fragments of the antibody. Other enzymes that cleave IgG molecules in this region include cathepsin L, plasmin, and metalloproteases.

Monoclonal antibodies in liquid formulation undergo non-enzymatic hydrolysis when stored at 5° C. for prolonged periods of time yielding Fab+Fc and Fab fragments (Jiskoot, W. et al. (1990) Pharm. Res. 7(12):1234-41; Alexander, A. J. and D. E. Hughes (1995) Anal. Chem. 67(20):3626-32; Cordoba, A. J. et al. (2005) J. Chromatogr. B Analyt. Technol. Biomed. Life Sci. 818(2):115-21; Liu, H. et al. (2006) J. Chrom. B Analyt. Technol. Biomed. Life Sci. 837:35-43; and Cohen, S. L. et al. (2007) J. Am. Chem. Soc. 129(22):6976-7). Fragmentation, typically monitored by size exclusion chromatography (SEC), increases at extreme pH conditions and high temperatures (Cohen, S. L. et al. (2007) J. Am. Chem. Soc. 129(22):6976-7). Cleavage occurred at multiple peptide bonds across the heavy chain region sequence Ser-Cys-Asp-Lys-Thr-His-Thr-Cys. Cleavage across the heavy chain sequence Cys-Asp-Lys-Thr-His-Thr-Cys resulted in the corresponding ladder of Fab fragment (48 kDa), whereas, cleavage between the Ser-Cys residues occurred via a beta elimination mechanism and resulted in heavy and light chain fragments (23 kDa).

Metal-induced fragmentation in the hinge region of an IgG molecule containing a kappa light chain was demonstrated in the recombinant monoclonal antibody, Campath (Smith, M. A. et al. (1996) Int. J. Pept. Protein Res. 48(1):48-55). Smith et al. reported copper mediated fragmentation at slightly alkaline pH and cleavage was specifically localized between the lysine and threonine residues in the hinge region of the heavy chain sequence Ser-Cys-Asp-Lys-Thr-His-Thr-Cys. The mechanism of cleavage was not revealed by the authors, however, cleavage was reduced at acidic conditions of pH 5-6.

A need remains to determine the parameters that surround fragmentation of antibody molecules in order to provide stable compositions (e.g., formulations) and methods for preventing cleavage of antibodies in their formulation during processing and storage.

For example, a need remains for an aqueous pharmaceutical formulation comprising an antibody, or fragment thereof, which is suitable for therapeutic use to inhibit or counteract detrimental IL-12 and/or IL-23 activity and which has an enhanced stability during processing and long term storage and which has enhanced resistance to fragmentation of the lambda light chain.

### SUMMARY OF THE INVENTION

The invention provides, in a first aspect, aqueous formulations comprising an antibody, or antigen binding portion thereof, that comprises a lambda chain, for example, an antibody that is suitable for therapeutic use to inhibit or counteract detrimental IL-12 and/or IL-23 activity and having improved properties as compared to art-recognized formulations. For example, the formulations of the invention have a shelf life of at least 24 months, e.g., in a liquid state or solid state. In another embodiment, the formulations of the invention maintain stability following at least 5 freeze/thaw cycles of the formulation.

The invention provides, in a second aspect, compositions and methods for inhibiting fragmentation of immunoglobulins comprising a lambda light chain based on the observation that iron, in the presence of histidine, results in increased fragmentation of an antibody containing a lambda light chain due to a specific cleavage in the hinge region. The presence of histidine alone in the formulation had no effect on the fragmentation. The level of fragmentation was dose dependent with regard to both iron and histidine levels. The elevated levels of fragmentation caused by iron and histidine were not observed in antibodies containing a kappa light chain. The lambda chain-containing antibody is cleaved at residues that are present in the hinge region, in the vicinity of the disulfide bond joining the light chain and the heavy chain.

In the first aspect, the invention provides a stable formulation comprising a molecule comprising at least a portion of a lambda light chain and a buffer system comprising histidine, wherein said formulation is substantially free of metal.

In an embodiment, the metal is Fe2+ or Fe3+. In another embodiment, the metal is Cu2+ or Cu1+.

In another embodiment, the invention further provides a stable formulation comprising a therapeutically effective amount of a molecule comprising a lambda light chain in a buffered solution comprising histidine with a pH of about 5 to about 7, wherein metal is present in a concentration that does not result in cleavage of the lambda light chain in the presence of histidine.

In another embodiment, the invention further provides a stable formulation comprising a molecule comprising at least a portion of a lambda light chain, a buffer system comprising imidazole, and a metal, wherein the molecule is not cleaved within the hinge region in the presence of a metal.

In an embodiment, the formulation is substantially free of metal following subjection to at least one procedure selected from the group consisting of filtration, buffer exchange, chromatography and resin exchange. In one embodiment, the buffer exchange comprises dialysis with a buffer selected from the group consisting of a buffer comprising histidine, a buffer comprising citrate and phosphate and a buffer comprising imidazole.

In an embodiment, the metal is present at a concentration of, for example, less than about 5,060 parts per billion (ppb), less than about 1,060 ppb, less than about 560 ppb, less than about 310 ppb, less than about 160 ppb, less than about 110 ppb and less than about 70 ppb. In a particular embodiment, the metal is present at a concentration of less than about 160 ppb, and more preferably at a concentration of less than about 70 ppb.

In an embodiment, the formulation comprises a molecule comprising a lambda light chain and at least one additional excipient selected from the group consisting of a polyol and a surfactant. In one embodiment, the formulation further comprises a stabilizer. In one embodiment, the formulation further comprises mannitol, polysorbate 80 and methionine. In one embodiment, the formulation further comprises a citrate buffer or a phosphate buffer. In one embodiment, the pH is about 5 or less. In another embodiment, the formulation comprises (a) 1-10% mannitol, (b) 0.001%-0.1% polysorbate-80 and (c) a buffer system comprising 1-100 mM histidine and 1-50 mM methionine, with a pH of 5 to 7. In yet another embodiment, the formulation comprises (a)₂₋₆% mannitol, (b) 0.005-0.05% polysorbate-80 and (c) a buffer system comprising 5-50 mM histidine and 5-20 mM methionine, with a p1-1 of 5 to 7. In a particular embodiment, the formulation comprises (a) about 4% mannitol, (b) about 0.01% polysorbate-80 and (c) a buffer system comprising about 10 mM histidine and about 10 mM methionine, with a pH of about 6.

In an embodiment, the invention provides an aqueous pharmaceutical formulation comprising (a) 1-250 mg/ml of a human antibody that binds to an epitope of a p40 subunit of IL-12/IL-23, (b) 1-10% mannitol, (c) 0.001%-0.1% polysorbate-80, (d) 1-50 mM methionine, and (e) 1-100 mM histidine, with a pH of 5 to 7, wherein the formulation is substantially free of metal.

In an embodiment, the pharmaceutical formulation does not have a conductivity of less than about 2.5 mS/com. In another embodiment, the pharmaceutical formulation is not the formulation used in Example 9 of U.S. Pat. No. 6,914,128.

In an embodiment, the molecule is a monoclonal antibody, or antigen binding portion thereof. In various embodiments, the concentration of the antibody, or antigen binding portion thereof, is, e.g., between about 1 and about 250 mg/ml, between about 40 and about 200 mg/ml, or is about 100 mg/ml.

In an embodiment, the antibody is a human antibody, or antigen binding portion thereof, capable of binding to an epitope of a p40 subunit of IL-12/IL-23. In an embodiment, the human antibody, or antigen-binding portion thereof, is capable of binding to the epitope of the p40 subunit when the p40 subunit is bound to a p35 subunit of IL-12. In another embodiment, the human antibody, or antigen-binding portion thereof, is capable of binding to the epitope of the p40 subunit when the p40 subunit is bound to a p19 subunit of IL-23. In yet another embodiment, the human antibody, or antigen-binding portion thereof, is capable of binding to the epitope of the p40 subunit when the p40 subunit is bound to the p35 subunit of IL-12 and also when the p40 subunit is bound to a p19 subunit of IL-23. In a particular embodiment, the human antibody, or antigen binding portion thereof, binds to an epitope of the p40 subunit of IL-12/IL-23 to which an antibody selected from the group consisting of Y61 and J695 binds.

In a particular embodiment, the invention still further provides an aqueous pharmaceutical formulation comprising (a) about 100 mg/ml of a human antibody that binds to an epitope of a p40 subunit of IL-12/IL-23, (b) about 4% mannitol, (b) about 0.01% polysorbate-80, (c) about 10 mM methionine, and (d) 10 mM histidine, with a pH of about 6.

In an embodiment, the human antibody, or antigen binding portion thereof, dissociates from the p40 subunit of IL-12/IL-23 with a K_{d} of 1×10⁻¹⁰ M or less or a k_{off} rate constant of 1×10⁻³ s⁻¹ or less, as determined by surface plasmon resonance.

In an embodiment, the human antibody, or antigen binding portion thereof, neutralizes the biological activity of the p40 subunit of IL-12/IL-23. In an embodiment, the human antibody, or antigen binding portion thereof neutralizes the biological activity of IL-12. In a particular embodiment, the neutralization of IL-12 function is achieved by interaction of the human antibody, or fragment thereof, with the p40 subunit of IL-12. In a particular embodiment, the human antibody, or an antigen binding portion thereof, inhibits phytohemagglutinin blast proliferation in an in vitro PHA assay with an IC₅₀ of 1×10⁻⁹M or less, or which inhibits human IFNγ production with an IC₅₀ of 1×10⁻¹⁰ M or less. In another embodiment, the human antibody, or binding portion thereof, neutralizes the biological activity of IL-23. In a particular embodiment the neutralization of IL-23 function is achieved by interaction of the human antibody, or fragment thereof, with the p40 subunit of IL-23.

In an embodiment, the human antibody, or antigen binding portion thereof, has a heavy chain CDR3 comprising the amino acid sequence of SEQ ID NO: 1 and a light chain CDR3 comprising the amino acid sequence of SEQ ID NO: 2. In another embodiment, the human antibody, or antigen binding portion thereof, has a heavy chain CDR2 comprising the amino acid sequence of SEQ ID NO: 3 and a light chain CDR2 comprising the amino acid sequence of SEQ ID NO: 4. In another embodiment, the human antibody, or antigen binding portion thereof, has a heavy chain CDR1 comprising the amino acid sequence of SEQ ID NO: 5 and a light chain CDR1 comprising the amino acid sequence of SEQ ID NO: 6. In yet another embodiment, the human antibody, or antigen binding portion thereof, has heavy chain variable region comprising the amino acid sequence of SEQ ID NO: 7 and a light chain variable region comprising the amino acid sequence of SEQ ID NO: 8. In a particular embodiment, the human antibody is the antibody J695, or an antigen binding portion thereof.

In an embodiment, the formulation has a shelf life of at least 24 months. In another embodiment, the formulation maintains stability following at least 5 freeze/thaw cycles of the formulation.

In an embodiment, the formulation further comprises an additional agent, e.g., an additional therapeutic agent.

In an embodiment, the additional therapeutic agent is selected from the group consisting of budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β monoclonal antibody, an anti-IL-1 receptor antibody, an anti-IL-6 monoclonal antibody, an anti-IL-6 receptor antibody, a growth factor, an elastase inhibitor, a pyridinyl-imidazole compound, an antibody or agonist of TNF, LT, IL-1, IL-2, IL-6, IL-7, IL-8, IL-15, IL-16, IL-17, IL-18, EMAP-II, GM-CSF, FGF, and PDGF, an antibody to CD2, CD3, CD4, CD8, CD20, CD25, CD28, CD30, CD40, CD45, CD69, CD90 or ligand thereof, methotrexate, FK506, rapamycin, mycophenolate mofetil, leflunomide, an NSAID, ibuprofen, prednisolone, a phosphodiesterase inhibitor, an S1P1 agonist, a bcl-2 inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα converting enzyme inhibitor, a T-cell signalling inhibitor, a metalloproteinase inhibitor, an angiotensin converting enzyme inhibitor, a soluble cytokine receptor, soluble p55 TNF receptor, soluble p75 TNF receptor, sIL-1RI, sIL-1RII, sIL-6R, an antiinflammatory cytokine, IL-4, IL-10, IL-11, IL-13, and TGFβ.

In another embodiment, the additional therapeutic agent is selected from the group consisting of an anti-TNF antibody and antibody fragments thereof, a TNFR-Ig construct, a TACE inhibitor, a PDE4 inhibitor, a corticosteroid, budenoside, dexamethasone, sulfasalazine, 5-aminosalicylic acid, olsalazine, an IL-1β converting enzyme inhibitor, IL-1ra, a tyrosine kinase inhibitor, a 6-mercaptopurine, and IL-11.

In yet another embodiment, the additional therapeutic agent is selected from the group consisting of methylprednisolone, cyclophosphamide, 4-aminopyridine, tizanidine, interferon-β1a, interferon-β1b, Copolymer 1, hyperbaric oxygen, intravenous immunoglobulin, clabribine, a TACE inhibitor, a kinase inhibitor, sIL-13R, an anti-P7, and p-selectin glycoprotein ligand (PSGL).

In another embodiment, the invention further provides a stable formulation comprising a molecule comprising at least a portion of a lambda light chain, a buffer system comprising histidine, and a metal chelator, wherein the molecule is not cleaved within the hinge region or is cleaved within the hinge region at a level which is less than the level of cleavage observed in the absence of the metal chelator.

In an embodiment, the metal is Fe2+ or Fe3+. In another embodiment, the metal is Cu2+ or Cu1+.

In an embodiment, the metal chelator is selected from the group consisting of citrate, a siderophore, calixerenes, an aminopolycarboxylic acid, a hydroxyaminocarboxylic acid, an N-substituted glycine, a 2-(2-amino-2-oxoethyl)aminoethane sulfonic acid (BES), a bidentate, tridentate or hexadentate iron chelator, a copper chelator, and derivatives, analogues, and combinations thereof. In a preferred embodiment, the metal chelator is desferrioxamine.

In the second aspect, the invention provides methods for inhibiting or preventing cleavage of a molecule comprising at least a portion of a lambda light chain in a histidine containing formulation, the method comprising the step of inhibiting or preventing the ability of metals to cleave the molecule. In an embodiment, the inhibiting or preventing comprises including at least one metal chelator in the formulation. In another embodiment, the inhibiting or preventing comprises subjecting the molecule to at least one procedure selected from the group consisting of filtration (e.g., ultrafiltration and diafiltration), buffer exchange, chromatography, and resin exchange. In one embodiment, the buffer exchange comprises dialysis with a buffer selected from the group consisting of a buffer comprising histidine, a buffer comprising citrate and phosphate and a buffer comprising imidazole.

In still another embodiment, the inhibiting or preventing comprises inhibiting or preventing cleavage by altering at least one amino acid in the lambda light chain or the heavy chain. In yet another embodiment, the inhibiting or preventing comprises inhibiting or preventing cleavage by altering the amino acid sequence in the lambda chain such that an amino acid sequence glutamic acid-cysteine-serine is changed. In yet another embodiment, the inhibiting or preventing comprises lowering the pH of the formulations towards more acidic levels, e.g., to a pH of 5 or less. In another embodiment, the inhibiting or preventing comprises including an additional buffer, such as a citrate buffer or a phosphate buffer, in the formulation. In an embodiment, the formulation comprises about 1-100 mM histidine, for example, about 10 mM histidine.

In an embodiment, the formulation comprises a level of iron that does not result in cleavage of the lambda chain containing antibody after 6 months at 25° C. or 40° C., e.g., iron is present at less than about 160 ppb.

In an embodiment, the molecule is present in a concentration range of about 1 mg/ml to about 300 mg/ml, for example about 2 mg/ml, for example about 7 mg/ml, for example about 100 mg/ml.

In an embodiment, the molecule is an immunoglobulin, for example, a monoclonal antibody. In a particular embodiment, the molecule is an anti-IL-12/23 antibody, for example, J695. In another embodiment, the antibody is an anti-CD-80 or and anti-IGF1,2 antibody.

In another embodiment, the molecule contains a hinge region selected from the group consisting of a DVD-Ig™, a Fab fragment, a F(ab′)₂ fragment, a chimeric antibody, a CDR-grafted antibody, a humanized antibody, a human antibody, a disulfide linked Fv, a single domain antibody, a multispecific antibody, a dual specific antibody, and a bispecific antibody. In an embodiment, the molecule comprises at least a portion of a heavy chain. In another embodiment, the portion of a heavy chain comprises the amino acid sequence serine-cysteine-aspartic acid-lysine (SCDK), or at least one modification that does not inhibit antibody binding. In another embodiment, the cleavage occurs in the hinge region between the serine and the cysteine residues. In yet another embodiment, the cleavage occurs between the cysteine and the aspartic acid residues.

In an embodiment, the metal is Fe2+ or Fe3+. In another embodiment, the metal is Cu2+ or Cu1+.

In an embodiment, the lambda light chain comprises the amino acid sequence of glutamic acid-cysteine-serine (ECS), or at least one modification that does not inhibit antibody binding. In another embodiment, the cleavage occurs in a hinge region of the lambda chain. In another embodiment, the cleavage occurs between the glutamic acid and the cysteine residues. In yet another embodiment, the cleavage occurs between the serine and the cysteine residues.

In an embodiment, the cleavage occurs at a temperature of about 2° C. to about 25° C., for example, about 2° C. to about 8° C. In an embodiment, the cleavage occurs at a pH of about 4 to about 8, for example about pH 5 to about 6.

In an embodiment, the at least one metal chelator is a siderophore selected from the group consisting of aerobactin, agrobactin, azotobactin, bacillibactin, N-(5-C3-L (5 aminopentyl)hydroxycarbamoyl)-propionamido)pentyl)-3(5-(N-hydroxyacetoamido)pentyl)carbamoyl)-proprionhydroxamic acid (deferoxamine, desferrioxamine or DFO or DEF), desferrithiocin, enterobactin, erythrobactin, ferrichrome, ferrioxamine B, ferrioxamine E, fluviabactin, fusarinine C, mycobactin, parabactin, pseudobactin, vibriobactin, vulnibactin, yersiniabactin, ornibactin, and derivatives, analogues, and combinations thereof (Roosenberg, J. M. et al. (2000) Studies and Syntheses of Siderophores, Microbial Iron Chelators, and Analogs as Potential Drug Delivery Agents. Current Medicinal Chem. 7: 159-197). In a preferred embodiment, the metal chelator is desferrioxamine.

In another embodiment, the at least one metal chelator is citrate or phosphate.

In another embodiment, the at least one metal chelator is an aminopolycarboxylic acid selected from the group consisting of ethylenediaminetetraacetic acid (EDTA), nitriloacetic acid (NTA), trans-diaminocyclohexane tetraacetic acid (DCTA), diethylenetriamine pentaacetic acid (DTPA), N-2-acetamido-2-iminodiacetic acid (ADA), aspartic acid, bis(aminoethyl)glycolether N,N,N′N′-tetraacetic acid (EGTA), glutamic acid, and N,N′-bis(2-hydroxybenzyl)ethylenediamine-N,N′-diacetic acid (HBED), and derivatives, analogues, and combinations thereof.

In another embodiment, the at least one metal chelator is a hydroxyaminocarboxylic acid selected from the group consisting of N-hydroxyethyliminodiacetic acid (HIMDA), N,N-bishydroxyethylglycine (bicine), and N-(trishydroxymethylmethyl)glycine (tricine), and derivatives, analogues, and combinations thereof.

In another embodiment, the at least one metal chelator is an N-substituted glycine, or derivative, analogue, or combination thereof. For example, the N-substituted glycine is selected from the group consisting of glycylglycine, and derivatives, analogues, and combinations thereof.

In another embodiment, the at least one metal chelator is 2-(2-amino-2-oxoethyl)aminoethane sulfonic acid (BES), or a derivative, analogue, and combination thereof.

In another embodiment, the at least one metal chelator is a calixarene, e.g., a macrocycle or cyclic oligomer based on a hydroxyalkylation product of a phenol and an aldehyde, or a derivative, analogue, or combination thereof (Gutsche, C. D. (1989) Calixarenes. Cambridge: Royal Society of Chemistry; Dharam, P. and Harjit, S. (2006) Syntheses, Structures and Interactions of Heterocalixarenes, Arcivoc.).

In another embodiment, the at least one metal chelator comprises a combination of DTPA and DEF. In another embodiment, the at least one metal chelator comprises a combination of EDTA, EGTA and DEF.

In another embodiment, the at least one metal chelator is a hydroxypyridine-derivate, a hydrazone-derivate, and hydroxyphenyl-derivate, or a nicotinyl-derivate, such as 1,2-dimethyl-3-hydroxypyridin-4-one (Deferiprone, DFP or Ferriprox); 2-deoxy-2-(N-carbamoylmethyl-[N′-2′-methyl-3′-hydroxypyridin-4′-one])-D-glucopyranose (Feralex-G), pyridoxal isonicotinyl hydrazone (P1H); 4,5-dihydro-2-(2,4-dihydroxyphenyl)-4-methylthiazole-4-carboxylic acid (GT56-252), 4-[3,5-bis(2-hydroxyphenyl)-[1,2,4]triazol-1-yl]benzoic acid (ICL-670); N,N′-bis(o-hydroxybenzyl)ethylenediamine-N,N′-diacetic acid (HBED), 5-chloro-7-iodo-quinolin-8-ol (clioquinol), or a derivative, analogue, or combination thereof.

In another embodiment, the at least one metal chelator is a copper chelator selected from the group consisting of triethylenetetramine (trientine), tetraethylenepentamine, D-penicillamine, ethylenediamine, bispyridine, phenantroline, bathophenanthroline, neocuproine, bathocuproine sulphonate, cuprizone, cis,cis-1,3,5,-triaminocyclohexane (TACH), tachpyr, and derivatives, analogues, and combinations thereof.

In another embodiment, the at least one metal chelator may be selected from the chelating agents, analogues and derivatives of agents described in the art, for example, that described in “Iron Chelators and Therapeutic Uses”, by Bergeron, R. et al., in Burger's Medicinal Chemistry and Drug Discovery, Sixth Edition, Volume 3: Cardiovascular Agents and Endocrines, edited by Abraham, D. J, John Wiley & Sons, Inc. 2003. Additionally, chelators may be selected from the chelating agents, analogues and derivatives of agents described in U.S. Pat. No. 6,083,966, in U.S. Pat. No. 6,521,652, in U.S. Pat. No. 6,525,080, in U.S. Pat. No. 6,559,315, in PCT/US2004/029318, in PCT/US2003/022012, in WO/2002/043722, and in WO 2004/007520.

In another embodiment, the formulation comprises at least one additional excipient selected from the group consisting of an amino acid, a sugar, a sugar alcohol, a buffer, a salt, and a surfactant.

In another embodiment, the formulation comprises at least one additional excipient selected from the group consisting of about 1 to about 60 mg/ml mannitol, about 1 to about 50 mM methionine, about 0.001% to about 0.5% (w/v) polysorbate 80, about 0.001% to about 1% (w/v) polyoxamer 188, about 1 to about 150 mM sodium chloride, about 1 to about 30 mM acetate, about 1 to about 30 mM citrate, about 1 to about 30 mM phosphate, and about 1 to about 30 mM arginine.

In another embodiment, the inhibiting or preventing of fragmentation comprises changing the pH of the formulation towards more acidic levels by adding acid, titrating or dialysis or various filtration processes known in the art to reduce pH such as, but not limited to, dialysis or tangential flow filtration.

In another embodiment, the inhibiting or preventing of fragmentation comprises use of specific buffers such as phosphate or citrate.

In another embodiment of the second aspect, the invention provides a method for detecting cleavage of a molecule comprising at least a portion of a lambda light chain in a histidine containing formulation, the method comprising the steps of including at least one metal chelator in the formulation and analyzing the at least a portion of the lambda light chain for cleavage.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 shows the hinge region of an antibody molecule.

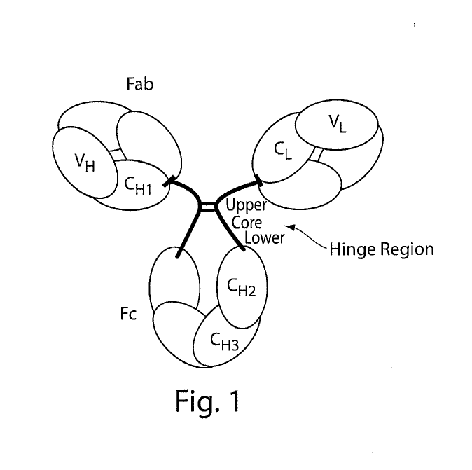

FIG. 2 shows fractionation (fractions 1-4) of the different species of J695 after size exclusion chromatography (SEC).

FIG. 3 shows evaluation of the different fractions from the SEC of FIG. 2 analyzed by SDS-PAGE showing a non-reducible (NR) species, a heavy chain (HC), a light chain (LC), and fragments of the HC(HC-Fc) in fraction 3 and the LC and HC-Fab in fraction 4.

FIG. 4 shows analysis by LC/ESI-MS of fraction 3 from FIG. 2, after deglycosylation, showing multiple cleavage sites on the HC in the hinge region. The peaks have been labeled from (a) to (e) and the identity of the peaks and cleavage site is provided in Table 1.

FIG. 5 shows analysis by MS of fraction 4 from FIG. 2 showing the corresponding Fab fragment in this fraction. Peaks are labeled from (f) to (j) and the identity of peaks and cleavage sites is provided in Table 1.

FIG. 6 shows analysis by MS of fraction 4 from FIG. 2, showing free LC from amino acid residues 1-215 and free HC from amino acid residues 1-217.

FIG. 7 shows analysis by CE-SDS of fraction 3 from FIG. 2 showing fragment 2 (Fab+Fc) whereas fraction 4 contained Fab and LC and HC fragments. Fragment 2 in the intact antibody is well resolved from other peaks.

FIG. 8 shows dialysis of J695 (Mab-lot 1) containing 500 ppb iron against citric acid buffer using a 10,000 MWCO membrane.

FIG. 9 shows different levels of metal salts (2.5, 10 and 50 ppm) spiked into a normal control lot of J695, incubated for 1 month at 40° C. and analyzed by CE-SDS.

FIG. 10 shows analysis by CE-SDS after incubation of J695 containing 500 ppb of iron with 1 mM of desferrioxamine, for 1 month at 40° C.

FIG. 11 shows a normal lot of J695 with no iron, after dialysis against water, and incubation with either histidine, iron, or both iron and histidine.

FIG. 12 shows a comparison of fragment 2 from FIG. 2 by ESI/LC-MS of stressed J695 containing 500 ppb of iron against a normal stressed lot.

FIG. 13 shows analysis of the corresponding Fab species revealing that the cleavage sites were comparable when stressed J695 containing iron was compared to a normal stressed lot.

FIG. 14 shows analysis of the LC and HC fragments revealing higher levels of fragments of the heavy (1-217) and light chains (1-215).

FIG. 15 shows investigation of iron-induced fragmentation of IgG molecules containing either a lambda or kappa light chain.

FIG. 16 shows the sequence of residues on lambda or kappa light chains and the bonds that are cleaved.

### DETAILED DESCRIPTION OF THE INVENTION

### I. Definitions

The term “antibody” broadly refers to any immunoglobulin (Ig) molecule comprised of four polypeptide chains, two heavy (H) chains and two light (L) chains, interconnected by disulfide bonds or any functional fragment, mutant, variant, or derivation thereof, which retains the essential epitope binding features of an Ig molecule. Such mutant, variant, or derivative antibody formats are known in the art, nonlimiting embodiments of which are discussed herein.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “Fc region” refers to the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (U.S. Pat. Nos. 5,648,260 and 5,624,821). The Fc portion of an antibody mediates several important effector functions, e.g., cytokine induction, antibody dependent cell mediated cytotoxicity (ADCC), phagocytosis, complement dependent cytotoxicity (CDC) and half-life/clearance rate of antibody and antigen-antibody complexes. Certain human IgG isotypes, particularly IgG1 and IgG3, mediate ADCC and CDC via binding to FcγRs and complement C1q, respectively. The dimerization of two identical heavy chains of an immunoglobulin is mediated by the dimerization of CH3 domains and is stabilized by the disulfide bonds within the hinge region (Huber et al. (1976) Nature 264:415-20; Thies et al. (1999) J. Mol. Biol. 293:67-79). Mutation of cysteine residues within the hinge regions to prevent heavy chain-heavy chain disulfide bonds destabilizes dimeration of CH3 domains. Residues responsible for CH3 dimerization have been identified (Dall'Acqua (1998) Biochem. 37:9266-73). Therefore, it is possible to generate a monovalent half-Ig. Monovalent half Ig molecules have been found in nature for both IgG and IgA subclasses (Seligman (1978) Ann. Immunol. 129:855-70; Biewenga et al. (1983) Clin. Exp. Immunol. 51:395-400). A half Ig molecule may have certain advantages in tissue penetration due to its smaller size than that of a regular antibody. In one embodiment, at least one amino acid residue is replaced in the constant region of the binding protein of the invention, for example the Fc region, such that the dimerization of the heavy chains is disrupted, resulting in half Ig molecules. The light chain may be either a kappa or lambda type.

The term “antigen-binding portion” of an antibody or “antibody portion” includes fragments of an antibody that retain the ability to specifically bind to an antigen (e.g., hIL-12 and/or hIL-23). Such antibody embodiments may also be bispecific, dual specific, or multi-specific, e.g., it specifically binds to two or more different antigens. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds. (2001) Antibody Engineering, Springer-Verlag, New York. pp. 790. In addition, single chain antibodies also include “linear antibodies” comprising a pair of tandem Fv segments (VH-CH1-VH-CH1) which, together with complementary light chain polypeptides, form a pair of antigen binding regions (Zapata et al. (1995) Protein Eng. 8(10):1057-1062; U.S. Pat. No. 5,641,870).

Still further, an antibody or antigen-binding portion thereof may be part of a larger immunoadhesion molecules, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

The term “multivalent binding protein” refers to a binding protein comprising two or more antigen binding sites. In an embodiment, the multivalent binding protein is engineered to have three or more antigen binding sites, and is generally not a naturally occurring antibody. The term “multispecific binding protein” also refers to a binding protein capable of binding two or more related or unrelated targets. Dual variable domain (DVD-Ig™) binding proteins comprise two or more antigen binding sites and are tetravalent or multivalent binding proteins. DVD-Ig™s may be monospecific, i.e., capable of binding one antigen, or multispecific, i.e., capable of binding two or more antigens. DVD-Ig™binding proteins comprising two heavy chain DVD-Ig™ polypeptides and two light chain DVD-Ig™ polypeptides are referred to as DVD-Ig™ Each half of a DVD-Ig™ comprises a heavy chain DVD-Ig™ polypeptide, and a light chain DVD-Ig™ polypeptide, and two antigen binding sites. Each binding site comprises a heavy chain variable domain and a light chain variable domain with a total of 6 CDRs involved in antigen binding per antigen binding site.

The term “bispecific antibody” refers to full-length antibodies that are generated by quadroma technology (Milstein, C. and A. C. Cuello (1983) Nature 305(5934):537-40), by chemical conjugation of two different monoclonal antibodies (Staerz, U. D. et al. (1985) Nature 314(6012):628-31), or by knob-into-hole or similar approaches that introduce mutations in the Fc region (Holliger, P. et al. (1993) Proc. Natl. Acad. Sci. USA 90:6444-8.18), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen to which it binds.

The term “dual-specific antibody” refers to a full-length antibody that can bind two different antigens (or epitopes) in each of its two binding arms (a pair of HC/LC) (PCT Publication No. WO 02/02773). Accordingly, a dual-specific binding protein has two identical antigen binding arms, with identical specificity and identical CDR sequences, and is bivalent for each antigen to which it binds.

An immunoglobulin constant domain refers to a heavy or light chain constant domain. Human IgG heavy chain and light chain constant domain amino acid sequences are known in the art.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method. In an embodiment, the monoclonal antibody is produced by hybridoma technology.

The term “chimeric antibody” refers to an antibody that comprises heavy and light chain variable region sequences from one species and constant region sequences from another species, such as antibodies having murine heavy and light chain variable regions linked to human constant regions.

The term “CDR-grafted antibody” refers to an antibody that comprises heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “human antibody” includes antibodies having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations preferably are introduced using the “selective mutagenesis approach” described in U.S. Pat. No. 6,914,128, the entire contents of which are incorporated by reference herein. The human antibody can have at least one position replaced with an amino acid residue, e.g., an activity enhancing amino acid residue which is not encoded by the human germline immunoglobulin sequence. The human antibody can have up to twenty positions replaced with amino acid residues that are not part of the human germline immunoglobulin sequence. In other embodiments, up to ten, up to five, up to three or up to two positions are replaced. In a preferred embodiment, these replacements are within the CDR regions as described in detail below. However, the term “human antibody”, as used herein, is not intended to include antibodies in which CDR sequences derived from the germline of another mammalian species, such as a mouse, have been grafted onto human framework sequences. Methods for generation human or fully human antibodies are known in the art and include EBV transformation of human B cells, selection of human or fully human antibodies from antibody libraries prepared by phage display, yeast display, mRNA display or other display technologies, and also from mice or other species that are transgenic for all or part of the human Ig locus comprising all or part of the heavy and light chain genomic regions defined further above. Selected human antibodies may be affinity matured by art recognized methods including in vitro mutagenesis, preferably of CDR regions or adjacent residues, to enhance affinity for the intended target.

The phrase “recombinant human antibody” includes human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody”, as used herein, is intended to refer to an antibody that is substantially free of other antibodies having different antigenic specificities (e.g., an isolated antibody that specifically binds human IL-12 and/or IL-23, e.g., binds the p40 subunit of human IL-12/IL-23, is substantially free of antibodies that specifically bind antigens other than human IL-12 and IL-23). An isolated antibody that specifically binds human IL-12 and/or IL-23 may, however, have cross-reactivity to other antigens, such as human IL-12 and/or IL-23 molecules from other species. Moreover, an isolated antibody may be substantially free of other cellular material and/or chemicals.

A “neutralizing antibody”, as used herein (or an “antibody that neutralizes human IL-12 and/or IL-23 activity” or an “antibody that neutralizes the activity of the p40 subunit of IL-12/IL-23”), is intended to refer to an antibody whose binding to human IL-12 and/or IL-23 (e.g., binding to the p40 subunit of IL-12/IL-23) results in inhibition of the biological activity of human IL-12 and/or IL-23 (e.g., biological activity of the p40 subunit of IL-12/IL-23). This inhibition of the biological activity of human IL-12 and/or IL-23 can be assessed by measuring one or more indicators of human IL-12 and/or IL-23 biological activity, such as inhibition of human phytohemagglutinin blast proliferation in a phytohemagglutinin blast proliferation assay (PHA), or inhibition of receptor binding in a human IL-12 and/or IL-23 receptor binding assay (e.g., an interferon-gamma induction Assay). These indicators of human IL-12 and/or IL-23 biological activity can be assessed by one or more of several standard in vitro or in vivo assays known in the art, and described in U.S. Pat. No. 6,914,128 (e.g., Example 3 at column 9, line 31 through column 113, line 55), the entire contents of which are incorporated by reference herein.

The term “humanized antibody” refers to an antibody that comprises heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. Also a “humanized antibody” is an antibody or a variant, derivative, analog or fragment thereof that specifically binds to an antigen of interest and which comprises a framework (FR) region having substantially the amino acid sequence of a human antibody and a complementary determining region (CDR) having substantially the amino acid sequence of a non-human antibody.

The term “hinge region” means the portion of a heavy chain molecule that joins the CH1 domain to the CH2 domain. The hinge region comprises approximately 25 residues and is flexible, thus allowing the two N-terminal antigen binding regions to move independently. Hinge regions can be subdivided into three distinct domains: upper, middle, and lower hinge domains (Roux et al. (1998) J. Immunol. 161: 4083). Some altered antibody molecules have been made in which the number of cysteine residues in the hinge region is reduced to one to facilitate assembly of antibody molecules as it is only necessary to form a single disulfide bond. This also provides a specific target for attaching the hinge region either to another hinge region or to an effector or reporter molecule (U.S. Pat. No. 5,677,425). The number of cysteine residues in the antibody hinge has also been increased (U.S. Pat. No. 5,677,425). Other mutated antibodies have been constructed in which the IgG1 hinge region and the CH2 domain have been replaced with the human IgG3 hinge region. (WO 97/11370). These molecules contain 11 sulfhydryl groups for substitution of multiple haptens via thiol groups.

The light chain component of the Ig protein is encoded by 2 separate loci, Igκ (kappa) and Igλ (lambda). The proportion of antibodies containing κ or λ light chains varies considerably between different species, e.g., in mice the κ:λ ratio is 95:5, compared to 60:40 in humans. In humans, while almost all λ producing cells have both κ alleles rearranged, the proportion of κ and λ producing cells are similar (Hieter, et al. (1981) Nature 290: 368-72; US 20040231012). B-cells express surface immunoglobulin (Ig) either with κ or λ light chain, a choice which is termed isotype exclusion. Light chain V-J rearrangement occurs at the transition from pre B-11 to immature B cells, where the surrogate light chain associated with membrane Igμ (mu) is replaced by κ or λ light chain (Osmond, et al. (1998) Immunol. Today 19, 65-68). Although the timing of light chain rearrangement is essentially defined, the processes that activate light chain locus rearrangement are not fully understood. Kappa and λ rearrangements are independent events (Arakawa, et al. (1996) Int. Immunol. 8: 91-99), the activation of which may be affected by differences in the strength of their respective enhancers. A region believed to be important in the regulation of the accessibility of the human λ locus has been identified about 10 Kb downstream of Cλ7 (Glozak and Blomberg (1996) Mol. Immunol. 33: 427-38; Asenbauer and Klobeck (1996) Eur. J. Immunol. 26: 142-50). Functional comparisons in reporter gene assays identified a core enhancer region that is flanked by elements that can drastically reduce enhancer activity in pre-B cells (Glozak and Blomberg (1996)). Although transfection studies showed that the κ and λ 3′ enhancer regions appear to be functionally equivalent, other (functional) sequences flanking the core enhancer motifs are remarkably dissimilar. Targeted deletion of the κ 3′ enhancer in transgenic mice showed that this region is not essential for κ locus rearrangement and expression but is required to establish the κ:λ ratio (Gorman, et al. (1996) Immunity 5: 241-52).

The human Ig λ locus on chromosome 22q 11.2 is 1.1 Mb in size and typically contains 70 Vλ genes and 7 Jλ-Cλ gene segments (Frippiat, et al. (1995) Hum. Mol. Genet. 4: 983-91; Kawasaki, et al. (1997) Genome Res. 7: 260-61). About half of the Vλ genes are regarded as functional and Jλ-Cλ, 1, 2, 3 and 7 are active. The Vλ genes are organized in 3 clusters which contain distinct V gene family groups. There are 10 Vλ gene families, with the largest VλIII being represented by 23 members. In human peripheral blood lymphocytes, the most J-C proximal V gene segments in cluster A, from families I, II and III, are preferentially rearranged, with the contribution of the 2a2 VA, segment (Giudicelli, et al. (1997) Nucl. Acids Res. 25: 206-11.) being unusually high (Ignatovich, et al. (1997) J. Mol. Biol. 268: 69-77). All λ gene segments have the same polarity, which allows deletional rearrangement (Combriato and Klobeck (1991) Eur. J. Immunol. 21: 1513-22). Sequence diversity of the Igλ repertoire is provided mainly by WAX combination. Additional CDR3 diversity due to N (nonencoded)- or P (palindromic)-nucleotide additions at the V to J junction, although not as extensive as seen in IgH rearrangement, seems to be much more frequently used in humans than in mice (Foster, et al. (1997) Clin. Invest. 99, 1614-27; Ignatovich, PhD thesis, University of Cambridge, 1998; Bridges et al. (1995) J. Clin. Invest. 96: 831-41; Victor et al. (1994) J. Immunol. 152: 3467-75), where the TdT (terminal deoxyribonucleotide transferase) activity is down-regulated at the time of light chain rearrangement. An alignment of several λ light chain sequences is provided below, indicating that there is a consensus sequence of

| QPKAXPXVTLFPPSSEELQANKATLVCLXSDFYPGAVTVAWKADXSPVKXGVETTXPSKQ | | |
|---|---|---|
| SNNKYAASSYLSLTPEQWKSHRSYSCXVTHEGSTVEKTVAPXECS; | | |
| where X is A, N, T, S, I, V, G, K, Q, or R. | | |
| | | 1 60 |
| hC11 | (1) | QPKANPTVTLFPPSSEELQANKATLVCLISDFYPGAVTVAWKADGSPVKAGVETTKPSKQ |
| hC12 | (1) | QPKAAPSVTLFPPSSEELQANKATLVCLISDFYPGAVTVAWKADSSPVKAGVETTTPSKQ |
| hC13 | (1) | QPKAAPSVTLFPPSSEELQANKATLVCLISDFYPGAVTVAWKADSSPVKAGVETTTPSKQ |
| hC17 | (1) | QPKAAPSVTLFPPSSEELQANKATLVCLVSDFYPGAVTVAWKADGSPVKVGVETTKPSKQ |
| | | 61 105 |
| hC11 | (61) | SNNKYAASSYLSLTPEQWKSHRSYSCQVTHEGSTVEKTVAPTECS |
| hC12 | (61) | SNNKYAASSYLSLTPEQWKSHRSYSCQVTHEGSTVEKTVAPTECS |
| hC13 | (61) | SNNKYAASSYLSLTPEQWKSHRSYSCQVTHEGSTVEKTVAPTECS |
| hC17 | (61) | SNNKYAASSYLSLTPEQWKSHRSYSCRVTHEGSTVEKTVAPAECS |

Human antibody kappa chains have been classified into four subgroups on the basis of invariant amino acid sequences (see, for example, Kabat et al. (1991), Sequences of Proteins of Immunological Interest (4th ed.), published by The U.S. Department of Health and Human Services). There appear to be approximately 80 human VK genes, but only one Subgroup IV VK gene has been identified in the human genome (see Klobeck, et al. (1985) Nucleic Acids Research, 13:6516-6528). The nucleotide sequence of Hum4VL is set forth in Kabat et al. (1991), supra. The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190:382-391; Kabat, E. A. et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within a antibody variable sequence. There are three CDRs in each of the variable regions of the heavy chain and the light chain, which are designated CDR1, CDR2 and CDR3, for each of the variable regions. The exact boundaries of these CDRs have been defined differently according to different systems. The system described by Kabat (Id.) not only provides an unambiguous residue numbering system applicable to any variable region of an antibody, but also provides precise residue boundaries defining the three CDRs. These CDRs may be referred to as Kabat CDRs. Chothia et al. found that certain sub-portions within Kabat CDRs adopt nearly identical peptide backbone conformations, despite having great diversity at the level of amino acid sequence (Chothia et al. (1987) Mol. Biol. 196:901-917; Chothia et al. (1989) Nature 342:877-883) These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45. Still other CDR boundary definitions may not strictly follow one of the herein described systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The term “chelator” broadly refers to an agent that binds to or forms complexes with metal ions. In an embodiment, such binding or complex formation includes one or more atoms of the metal chelator. The binding and complex formation can be any form and combination of bonds, e.g., covalent, dative, or ionic. In one embodiment, a chelator binds to or forms a complex with the metal ions and thereby sequesters the metal ions. Derivatives, analogues, and combination formats of metal chelators are known in the art, non-limiting embodiments of which are discussed below.

The term “normal stressed lot” means a lot that has been incubated at an elevated temperature (typically 25° C. or 40° C.) in the absence of metals. For example, in a normal stressed lot, cleavage of a molecule comprising at least a portion of a lambda light chain (e.g., an antibody) may occur in the hinge region, such as, for example, at multiple peptide bonds across the heavy chain region sequence Ser-Cys-Asp-Lys-Thr-His-Thr-Cys.

The phrase “substantially free of metal” or the “concentration of metal in the formulation that does not result in cleavage of the lambda light chain” refers to a concentration of metal in the formulation that is sufficiently low (e.g., less than about 160 ppb, preferably less than about 110 and more preferably less than about 70 ppb at a temperature of, e.g., 25° C. or 40° C.) such that a normal or acceptable level of fragmentation or cleavage of a lambda light chain containing antibody present in the formulation is observed, e.g., the cleavage level observed in a corresponding normal stressed lot, e.g., about 0.5% fragmentation. For example, the concentration of metal in the formulation is such that only less than about 0.1%, 0.2%, 0.3%, 0.4% or 0.5% of fragmentation or cleavage in the lambda light chain (e.g., the hinge region of the lambda chain) is observed. The level of fragmentation or cleavage of a lambda light chain containing antibody in a formulation may be determined, for example, by SEC, capillary electrophoresis and/or mass spectrometry.

The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

The term “pharmaceutical formulation” refers to preparations which are in such form as to permit the biological activity of the active ingredients to be unequivocally effective, and which contain no additional components which are significantly toxic to the subjects to which the formulation would be administered. “Pharmaceutically acceptable” excipients (e.g., vehicles, additives) are those which can reasonably be administered to a subject mammal to provide an effective dose of the active ingredient employed.

A “stable” formulation is one in which the antibody therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Preferably, the formulation is stable for 24 months at between 2 and 8° C. Further, the formulation is preferably stable for at least 18 months, and preferably for 24 months, at between −20 and −80° C. Furthermore, the formulation is preferably stable following freezing (to, e.g., −80° C.) and thawing (at, e.g., 25 to 37° C.) of the formulation, hereinafter referred to as a “freeze/thaw cycle.” Preferably, the formulation is stable following at least five freeze/thaw cycles.

An antibody “retains its physical stability” in a pharmaceutical formulation if it shows substantially no signs of aggregation, precipitation and/or denaturation upon visual examination of color and/or clarity, or as measured by UV light scattering or by size exclusion chromatography.

An antibody “retains its chemical stability” in a pharmaceutical formulation, if the chemical stability at a given time is such that the antibody is considered to still retain its biological activity as defined below. Chemical stability can be assessed by detecting and quantifying chemically altered forms of the antibody. Chemical alteration may involve size modification (e.g., clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g., occurring as a result of deamidation), which can be evaluated by ion-exchange chromatography, for example.

An antibody “retains its biological activity” in a pharmaceutical formulation, if the antibody in a pharmaceutical formulation is biologically active for its intended purpose. For example, biological activity is retained if the biological activity of the antibody in the pharmaceutical formulation is within about 30%, about 20%, or about 10% (within the errors of the assay) of the biological activity exhibited at the time the pharmaceutical formulation was prepared (e.g., as determined in an antigen binding assay).

“Isotonic” can mean, for example, that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example. A “tonicity agent” is a compound which renders the formulation isotonic.

A “polyol” is a substance with multiple hydroxyl groups, and includes sugars (reducing and nonreducing sugars), sugar alcohols and sugar acids. Preferred polyols herein have a molecular weight which is less than about 600 kD (e.g., in the range from about 120 to about 400 kD). A “reducing sugar” is one that contains a hemiacetal group that can reduce metal ions or react covalently with lysine and other amino groups in proteins and a “nonreducing sugar” is one that does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof. The polyol may also act as a tonicity agent. In one embodiment of the invention, one ingredient of the formulation is mannitol in a concentration of about 10 to about 100 mg/ml (e.g., 1-10%). In a particular embodiment of the invention, the concentration of mannitol is 30 to 50 mg/ml (e.g., 3-5%). In a preferred embodiment of the invention, the concentration of mannitol is about 40 mg/ml (e.g., 4%).

As used herein, “buffer” refers to a buffered solution that resists changes in pH by the action of its acid-base conjugate components. A buffer used in this invention has a pH in the range from about 4.0 to about 4.5, about 4.5 to about 5.0, about 5.0 to about 5.5, about 5.5 to about 6, about 6.0 to about 6.5, about 5.7 to about 6.3, about 6.5 to about 7.0, about 7.5 to about 8.0. In one embodiment, a buffer of the invention has a pH of about 5 or less. In one embodiment, a buffer of the invention has a pH of about 6. Examples of buffers that will control the pH in this range include acetate (e.g. sodium acetate), succinate (such as sodium succinate), gluconate, histidine, methionine, citrate, phosphate, imidazole, and other organic acid buffers. In one embodiment of the invention, the buffer system comprises histidine. In a particular embodiment of the invention, the buffer system comprises histidine and methionine. In one embodiment, the buffer system comprises 1-50 mM histidine (e.g., between 5-40 mM, between 10-30 mM, or between 10-20 mM) with a pH of 5-7, e.g., about 5 or about 6. In a preferred embodiment, the buffer system of the invention comprises 1-50 mM histidine (e.g., between 5-40 mM, between 10-30 mM, or between 10-20 mM) and 1-50 mM methionine (e.g., between 5-40 mM, between 10-30 mM, or between 10-20 mM) with a pH of 5-7, e.g., about 5 or about 6. In one embodiment, the buffer system comprises about 10 mM histidine, with a pH of about 6. In one embodiment, the buffer system comprises about 10 mM histidine, with a pH of about 5 or less. In a particularly preferred embodiment of the invention, the buffer comprises about 10 mM histidine and about 10 mM methionine with a pH of about 6. In another preferred embodiment of the invention, the buffer comprises about 10 mM histidine and about 10 mM methionine with a pH of about 5 or less.

In another embodiment of the invention, the buffer system comprises histidine and phosphate. In a particular embodiment, the buffer system comprises histidine at a concentration of between 1-50 mM (e.g., between 5-40 mM, between 10-30 mM, or between 10-20 mM) and preferably about 10 mM, and phosphate (e.g., sodium hydrogen phosphate) at a concentration of between 1-60 mM (e.g., between 10-50 mM, between 20-40 mM) and preferably 30 mM. In a preferred embodiment, the buffer system comprises histidine, methionine and phosphate, for example, the buffer system comprises histidine at a concentration of between 1-50 mM (e.g., between 5-40 mM, between 10-30 mM, or between 10-20 mM) and preferably about 10 mM, methionine at a concentration of between 1-50 mM (e.g., between 5-40 mM, between 10-30 mM, or between 10-20 mM) and preferably about 10 mM, and phosphate at a concentration of between 1-60 mM (e.g., between 10-50 mM, between 20-40 mM, or between 20-30 mM) and preferably about 30 mM.

In another embodiment, the buffer system comprises histidine and citrate. In a particular embodiment, the buffer system comprises histidine at a concentration of between 1-50 mM (e.g., between 5-40 mM, between 10-30 mM, or between 10-20 mM) and preferably about 10 mM, and citrate at a concentration of between 1-60 mM (e.g., between 10-50 mM, or between 20-40 mM) and preferably about 30 mM. In a preferred embodiment, the buffer system comprises histidine, methionine and citrate, for example, the buffer system comprises histidine at a concentration of between 1-50 mM (e.g., between 5-40 mM, between 10-30 mM, or between 10-20 mM) and preferably about 10 mM, methionine at a concentration of between 1-50 mM (e.g., between 5-40 mM, between 10-30 mM, or between 10-20 mM) and preferably about 10 mM, and citrate at a concentration of between 1-60 mM (e.g., between 10-50 mM, or between 20-40 mM) and preferably about 30 mM.

In yet another rembodiment, the buffer system comprises imidazole. In one embodiment, the buffer system comprises imidazole at a concentration of between 1-50 mM, between 5-40 mM, between 5-30 mM, between 10-30 mM, between 10-20 mM, and preferably, e.g., 10 mM. In a preferred embodiment, the buffer system comprises imidazole and methionine, e.g., imidazole at a concentration of between 1-50 mM (e.g., between 5-40 mM, between 5-30 mM, between 10-30 mM, or between 10-20 mM) and preferably 10 mM, and methionine at a concentration of between 1-50 mM (e.g., between 5-40 mM, between 10-30 mM, or between 10-20 mM) and preferably about 10 mM.

In still another embodiment, the buffer system comprises phosphate and citrate, e.g., phosphate (e.g., sodium hydrogen phosphate) at a concentration of between 1-50 mM (e.g., between 5-40 mM, between 5-30 mM, between 10-20 mM) and preferably 10 mM, and citrate (citric acid) at a concentration of between 1-50 mM (e.g., between 5-40 mM, between 5-30 mM, between 10-20 mM) and preferably 10 mM.

In any of the foregoing buffer systems, the pH is preferably between about 2 and 7, between about 3 and 7, between about 4 and 7, e.g., about 5 or less (e.g., between about 2 and 5, between about 2.5 and 5, between about 3 and 5, between about 3.5 and 5, between about 4.0 and 5 or between about 4.5 and 5) or about 6.

In a pharmacological sense, in the context of the present invention, a “therapeutically effective amount” or “effective amount” of an antibody refers to an amount effective in the prevention or treatment of a disorder for the treatment of which the antibody is effective. A “disorder” is any condition that would benefit from treatment with the antibody. This includes chronic and acute disorders or diseases including those pathological conditions which predisposes the subject to the disorder in question.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

“Treatment” refers to both therapeutic treatment and prophylactic or preventative measures. Those in need of treatment include those already with the disorder as well as those in which the disorder is to be prevented.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable carrier” is art recognized and includes a pharmaceutically acceptable material, composition or vehicle, suitable for administration to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient.

The phrase “human interleukin 12” or “human IL-12” (abbreviated herein as hl L-12, or IL-12), as used herein, includes a human cytokine that is secreted primarily by macrophages and dendritic cells. The term includes a heterodimeric protein comprising a 35 kD subunit (p35) and a 40 kD subunit (p40) which are both linked together with a disulfide bridge. The heterodimeric protein is referred to as a “p70 subunit”. The structure of human IL-12 is described further in, for example, Kobayashi, et al. (1989) J. Exp Med. 170:827-845; Seder, et al. (1993) Proc. Natl. Acad. Sci. 90:10188-10192; Ling, et al. (1995) J. Exp Med. 154:116-127; Podlaski, et al. (1992) Arch. Biochem. Biophys. 294:230-237; and Yoon et al. (2000) EMBO Journal 19(14): 3530-3541. The term human IL-12 is intended to include recombinant human IL-12 (rh IL-12), which can be prepared by standard recombinant expression methods.

The phrase “human interleukin 23” or “human IL-23” (abbreviated herein as hIL-23, or IL-23), as used herein, includes a human cytokine that is secreted primarily by macrophages and dendritic cells. The term includes a heterodimeric protein comprising a 19 kD subunit (p19) and a 40 kD subunit (p40) which are both linked together with a disulfide bridge. The heterodimeric protein is referred to as a “p40/p19” heterodimer. The structure of human IL-23 is described further in, for example, Beyer et al. (2008) J. Mol. Biol. 382:942-955; Lupardus et al. (2008) J. Mol. Biol. 382:931-941. The term human IL-23 is intended to include recombinant human IL-23 (rhIL-23), which can be prepared by standard recombinant expression methods.

The phrase “p40 subunit of human IL-12/IL-23” or “p40 subunit of human IL-12 and/or IL-23,” or “p40 subunit” as used herein, is intended to refer to a p40 subunit that is shared by human IL-12 and human IL-23. The structure of the p40 subunit of IL-12/IL-23 is described in, for example, Yoon et al. (2000) EMBO Journal 19(14): 3530-3541.

The term “activity” includes activities such as the binding specificity/affinity of an antibody for an antigen, for example, an anti-p40 antibody that binds to an IL-12 and/or IL-23 antigen and/or the neutralizing potency of an antibody, for example, an anti-p40 antibody whose binding to human IL-12 and/or human IL-23 inhibits the biological activity of human IL-12 and/or human IL-23, e.g. inhibition of PHA blast proliferation or inhibition of receptor binding in a human IL-12 receptor binding assay (see, e.g., Example 3 of U.S. Pat. No. 6,914,128).

The phrase “surface plasmon resonance” includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

### II. Compositions and Methods of the Invention

Using size exclusion chromatography (SEC), mass spectrometry (MS) and capillary electrophoresis (CE) to monitor fragmentation, a degradation pathway whereby both histidine and metal (either iron or copper) act together to fragment lambda light chain containing molecules was discovered. Both iron and histidine are needed to accelerate the kinetics of fragmentation in the hinge region of an antibody molecule at 40° C. Iron or histidine alone had little or no effect on accelerating the kinetics of fragmentation of the IgG molecule. Metal spiking studies conducted with a number of different metals showed that the presence of iron or copper in the antibody formulation results in cleavage of the antibody in a dose dependent manner. Chelation of iron with desferrioxamine, an iron specific chelator, blocked this fragmentation. Investigation of IgG molecules having either a lambda or a kappa chain show that this fragmentation mechanism is specific for molecules that contain a lambda chain. The kappa and lambda light chains differ in their C-terminal regions and the lambda light chain has an extra serine residue after the cysteine residue.

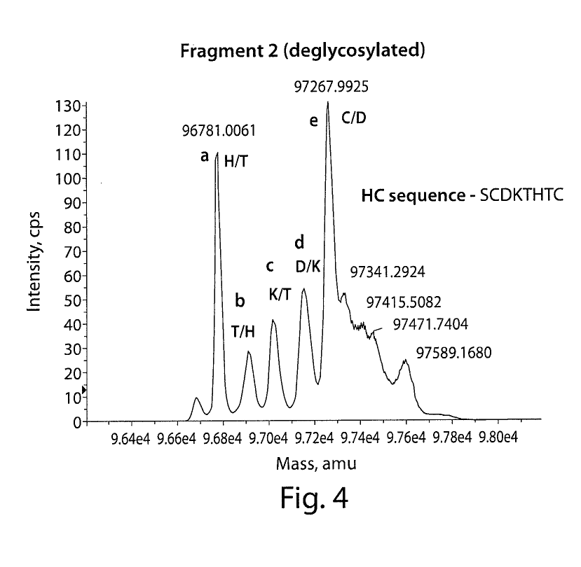

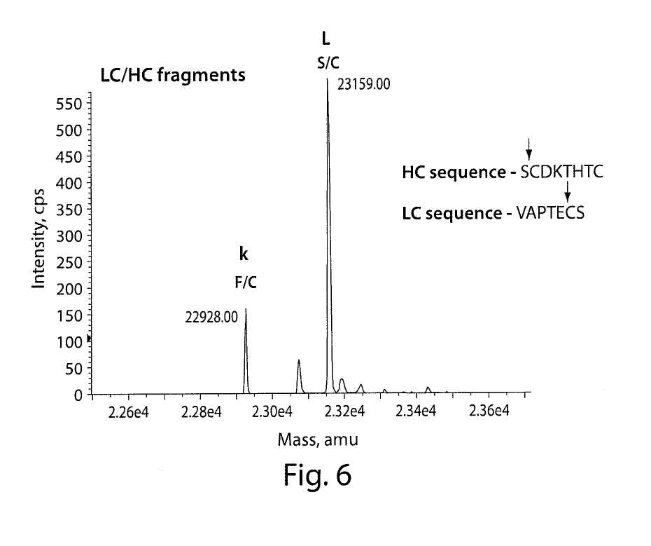

SEC was used to monitor aggregates and fragments and to fractionate fragments of antibody after incubation at elevated temperature for prolonged time. CE-SDS was used to not only accurately quantify fragments but to also quantify other degradation species. MS spectra of a normal stressed lot showed that the major cleavage sites on fragment 2 (Fab+Fc) are between residues C/D, D/K, K/T, T/H and H/T of the heavy chain (FIG. 4). Cleavage between the serine-217 and cysteine-218 residues (S/C) of the heavy chain was increased in iron and histidine containing formulations and consequently elevated levels of the HC fragment 1-217 are seen in the MS spectra (FIG. 6). Similar to the normal stressed lots, the corresponding Fab+Fc fragment beginning with cys-218 was not found. Instead, elevation of a Fab+Fc fragment that began with aspartic acid (cleavage between C/D) and a species that showed the addition of 27 Da to the aspartic acid fragment, was observed. Free LC (residues 1-217) was not observed in the MS spectra but elevated levels of LC cleaved between residues E/C giving fragment 1-215 that ended with glutamic acid were detected. These results show that iron induced cleavage was localized to residues around the disulfide bonds holding the HC and LC together.

Metal ions are known to catalyze the oxidation and degradation of proteins in different ways. They either react directly with thiol groups of cysteine residues (site specific) to produce radicals or they may react with oxygen to produce a number of reactive oxygen species such as the superoxide radical anion, hydroxyl radicals and hydrogen peroxide (Li, S. et al. (1995) Biotech. and Bioeng. 48:490-500; Li, S. et al. (1993) Pharm. Res. 10(11):1572-1579; Kocha, T. et al. (1997) BBA 1337:319-326). Reactive oxygen species (ROS) produced in the presence of metal ions and a reducing environment (DTT, ascorbate) will cleave the protein backbone (Kim, R. et al. (1985). While not wishing to be bound by any particular theory, it is possible that chelates of copper and histidine catalyze a variety of oxidations. Chelates of iron and histidine have been reported (Davison, A. J. (1968) J. Biol. Chem. 243(22):6064-6067; Lavanant, H. et al. (1999) Int. J. Mass Spectrom. 185/186/187:11-23). The lambda light chain has a free serine residue that is absent on the kappa chain. A recent report has shown that peptides ending with a C-terminal serine residue are efficiently hydrolyzed in the presence of metals (Yashiro, M. et al. (2003) Org. Biomol. Chem. 1:629-632).

In an embodiment, filtration methods include diafiltration, ultrafiltration, or a combination thereof. In an embodiment, buffer exchange methods include dialysis. In another embodiment, buffer exchange includes the use of desalting columns. In an embodiment, chromatography methods include the use of affinity chromatography such as protein A or weak cation exchange chromatography to capture the antibody.

In an embodiment, resin exchange methods include the use of Chelex-100 to bind and strip metals.

In an embodiment, amino acids in LC an HC are substituted, or deleted to inhibit metal and histidine related cleavage. Amino acids that may be substituted or deleted include the C-terminal serine residue present on the lambda light chain. Other residues include the serine residue adjacent to the cysteine residue on the heavy chain.

### III. Antibodies Suitable for Use in the Formulations of the Invention

The invention provides formulations comprising an antibody in a histidine buffered solution having a pH between about 5 and about 7 and having enhanced stability, preferably of at least about 24 months, e.g., at a temperature of 2-8° C. or at a temperature of between −20 and −180° C. In another embodiment of the invention, the claimed formulation remains stable following at least 5 freeze/thaw cycles. In a preferred embodiment, the amount of metal in the formulation is sufficiently low to prevent cleavage of the antibody, e.g., cleavage of the lambda light chain of the antibody. Preferably, the claimed formulation is free of metal. In another preferred embodiment, the formulation comprises a metal chelator, wherein the antibody is not cleaved or is cleaved less, e.g., within the hinge region of the lambda light chain, in the presence of a metal. In still another embodiment, the pharmaceutical formulation of the invention is suitable for single use sc injection.

Antibodies that can be used in the formulation include polyclonal, monoclonal, recombinant antibodies, single chain antibodies, hybrid antibodies, chimeric antibodies, humanized antibodies, or fragments thereof. Antibody-like molecules containing one or two binding sites for an antigen and a Fc-part of an immunoglobulin can also be used. In a preferred embodiment of the invention, antibodies used in the formulation comprise at least a portion of a lambda light chain. Preferred antibodies used in the formulations of the invention are human antibodies. In a preferred embodiment, the formulation contains an antibody which is an isolated human recombinant antibody, or an antigen-binding portion thereof. In another particular embodiment, the antibody is a lambda chain-containing antibody or antigen binding portion thereof.

In one aspect of the invention, the formulation contains a human antibody, e.g., human antibody comprising a lambda chain, that binds to an epitope of the p40 subunit of IL-12/IL-23. In one embodiment, the antibody binds to the p40 subunit when the p40 subunit is bound to the p35 subunit of IL-12. In one embodiment, the antibody binds to the p40 subunit when the p40 subunit is bound to the p19 subunit of IL-23. In one embodiment, the antibody binds to the p40 subunit when the subunit is bound to the p35 subunit of IL-12 and also when the p40 subunit is bound to the p19 subunit of 11-23. In a preferred embodiment, the antibody, or antigen-binding portion thereof, is an antibody like those described in U.S. Pat. No. 6,914,128, the entire contents of which are incorporated by reference herein. For example, in a preferred embodiment, the antibody binds to an epitope of the p40 subunit of IL-12 to which an antibody selected from the group consisting of Y61 and J695, as described in U.S. Pat. No. 6,914,128, binds. Especially preferred among the human antibodies is J695 as described in U.S. Pat. No. 6,914,128. Other antibodies that bind IL-12 and/or IL-23 and which can be used in the formulations of the invention include the human anti-IL-12 antibody C340, as described in U.S. Pat. No. 6,902,734, the entire contents of which are incorporated by reference herein.

In one embodiment, the formulation of the invention includes a combination of antibodies (two or more), or a “cocktail” of antibodies. For example, the formulation can include the antibody J695 and one or more additional antibodies.

In one aspect, the formulation of the invention contains J695 antibodies and antibody portions, J695-related antibodies and antibody portions, and other human antibodies and antibody portions with equivalent properties to J695, such as high affinity binding to hIL-12/IL-23 with low dissociation kinetics and high neutralizing capacity. For example, in one embodiment of the invention, the formulation contains a human antibody, or antigen-binding portion thereof, that dissociates from the p40 subunit of human IL-12/IL-23 with a K_{d} of 1.34×10⁻¹⁰ M or less or with a K_{off} rate constant of 1×10⁻³ s⁻¹ or less, as determined by surface plasmon resonance. Preferably, the antibody, or antigen-binding portion thereof, dissociates from the p40 subunit of human IL-12/IL-23 with a k_{off} rate constant of 1×10⁻⁴ s⁻¹ or less, and more preferably with a k_{off} rate constant of 1×10⁻⁵ S⁻¹ or less, or with a K_{d} of 1×10⁻¹⁰ M or less, and more preferably with a K_{d} of 9.74×10⁻¹¹ M or less.

The dissociation rate constant (K_{off}) of an IL-12/IL-23 antibody can be determined by surface plasmon resonance. Generally, surface plasmon resonance analysis measures real-time binding interactions between ligand (recombinant human IL-12 immobilized on a biosensor matrix) and analyte (antibodies in solution) by surface plasmon resonance (SPR) using the BIAcore system (Pharmacia Biosensor, Piscataway, N.J.). Surface plasmon analysis can also be performed by immobilizing the analyte (antibodies on a biosensor matrix) and presenting the ligand (recombinant IL-12/IL-23 in solution) (see, for example, assays described in Example 5 of U.S. Pat. No. 6,914,128, the contents of which are incorporated by reference herein). Neutralization activity of IL-12/IL-23 antibodies, or antigen binding portions thereof, can be assessed using one or more of several suitable in vitro assays (see for example, assays described in Example 3 of U.S. Pat. No. 6,914,128, the contents of which are incorporated by reference herein).

In another embodiment of the invention, the formulation contains a human antibody, or antigen-binding portion thereof, that neutralizes the biological activity of the p40 subunit of human IL-12/IL-23. In one embodiment, the antibody, or antigen-binding portion thereof, neutralizes the biological activity of free p40, e.g., monomer p40 or a p40 homodimer, e.g., a dimer containing two identical p40 subunits. In preferred embodiments, the antibody, or antigen-binding portion thereof, neutralizes the biological activity of the p40 subunit when the p40 subunit is bound to the p35 subunit 5′ of 11-12 and/or when the p40 subunit is bound to the p19 subunit of IL-23. In various embodiments, the antibody, or antigen-binding portion thereof, inhibits human IL-12-induced phytohemagglutinin blast proliferation in an in vitro PHA assay with an IC₅₀ of 1×10⁻⁷M or less, preferably with an IC₅₀ of 1×10⁻⁸ M or less, more preferably with an IC₅₀ of 1×10⁻⁹M or less, even more preferably with an IC₅₀ of 1×10⁻¹¹ M or less, and most preferably with an IC₅₀ of 1×10⁻¹¹ M or less. In other embodiments, the antibody, or antigen-binding portion thereof, inhibits human IL-12-induced human IFNγ production with an IC₅₀ of 1×10⁻¹⁰ M or less, preferably with an IC₅₀ of 1×10⁻¹¹ M or less, and more preferably with an IC₅₀ of 5×10⁻¹² M or less.

In yet another embodiment of the invention, the formulation contains a human antibody, or antigen-binding portion thereof, which has a heavy chain CDR3 comprising the amino acid sequence of SEQ ID NO: 1 and a light chain CDR3 comprising the amino acid sequence of SEQ ID NO: 2. In one embodiment, the human antibody, or antigen binding portion thereof, further has a heavy chain CDR2 comprising the amino acid sequence of SEQ ID NO: 3 and a light chain CDR2 comprising the amino acid sequence of SEQ ID NO: 4. In one embodiment, the human antibody, or antigen binding portion thereof, further has a heavy chain CDR1 comprising the amino acid sequence of SEQ ID NO: 5 and a light chain CDR1 comprising the amino acid sequence of SEQ ID NO: 6. In a particularly preferred embodiment, the antibody, or antigen binding portion thereof, has heavy chain variable region comprising the amino acid sequence of SEQ ID NO: 7, and a light chain variable region comprising the amino acid sequence of SEQ ID NO: 8. The antibody, or antigen binding portion thereof, of the formulations of the invention can comprise a heavy chain constant region selected from the group consisting of IgG1, IgG2, IgG3, IgG4, IgM, IgA and IgE constant regions. Preferably, the antibody heavy chain constant region is IgG1. In various embodiments, the antibody, or antigen binding portion thereof, is a Fab fragment, a F(ab′)₂ fragment, or a single chain Fv fragment.

Examples of lambda chain-containing antibodies, e.g., lambda chain-containing antibodies that may be included in formulations of the invention, are well known in the art and are understood to be encompassed by the invention. Examples of lambda chain-containing antibodies include, but are not limited to, the anti-IL-17 antibody Antibody 7 as described in International Application WO 2007/149032 (Cambridge Antibody Technology), the entire contents of which are incorporated by reference herein, the anti-IL-12/IL-23 antibody J695 (Abbott Laboratories), the anti-IL-13 antibody CAT-354 (Cambridge Antibody Technology), the anti-human CD4 antibody CE9y4PE (IDEC-151, clenoliximab) (Biogen IDEC/Glaxo Smith Kline), the anti-human CD4 antibody IDEC CE9.1/SB-210396 (keliximab) (Biogen IDEC), the anti-human CD80 antibody IDEC-114 (galiximab) (Biogen IDEC), the anti-Rabies Virus Protein antibody CR4098 (foravirumab), and the anti-human TNF-related apoptosis-inducing ligand receptor 2 (TRAIL-2) antibody HGS-ETR2 (lexatumumab) (Human Genome Sciences, Inc.).

### IV. Preparation of Formulations

The present invention features formulations (e.g., protein formulations and/or antibody formulations) having improved properties as compared to art-recognized formulations. For example, the formulations of the invention have an improved shelf life and/or stability as compared to art recognized formulations. In one embodiment, the formulations of the invention have a shelf life of at least 18 months, e.g., in a liquid state or in a solid state. In another embodiment, the formulations of the invention have a shelf life of at least 24 months, e.g., in a liquid state or in a solid state. In a preferred embodiment, the formulations of the invention have a shelf life of at least 24 months at a temperature of 2-8° C. In a preferred embodiment, the formulations of the invention have a shelf life of at least 18 months or of at least 24 months at a temperature of between about −20 and −80° C. In another embodiment, the formulations of the invention maintain stability following at least 5 freeze/thaw cycles of the formulation. In a preferred aspect, the formulations of the invention comprise a molecule, e.g., an antibody, comprising at least a portion of a lambda light chain, wherein the formulation provides enhanced resistance to fragmentation of the lambda light chain, e.g., reduced cleavage of the lambda light chain, as compared to art recognized formulations.

In a preferred aspect, the formulations of the invention are substantially free of metal. In a preferred embodiment, the formulations of the invention are substantially free of a metal selected from the group consisting of Fe2+ and Fe3+. In another preferred embodiment, the formulations of the invention are substantially free of a metal selected from the group consisting of Cu2+ and Cu1+. In a preferred embodiment, the formulations of the invention comprise an amount of metal that is sufficiently low to reduce or prevent cleavage of the lambda chain in the presence of histidine, e.g., the metal is present at a concentration of less than about 5,060 ppb, less than about 1,060 ppb, less than about 560 ppb, less than about 500 ppb, less than about 450 ppb, less than about 400 ppb, less than about 350 ppb, less than about 310 ppb, less than about 300 ppb, less than about 250 ppb, less than about 200 ppb, less than about 160 ppb, less than about 150 ppb, less than about 140 ppb, less than about 130 ppb, less than about 120 ppb, less than about 110 ppb, less than about 100 ppb, less than about 90 ppb, less than about 80 ppb, less than about 70 ppb, less than about 60 ppb, less than about 50 ppb, less than about 40 ppb, less than about 30 ppb, less than about 20 ppb, less than about 10 ppb, or less than about 1 ppb. In a preferred embodiment, the metal is present at a concentration of less than about 160 ppb. In a preferred embodiment, the metal is present at a concentration of less than about 110 ppb. In a particularly preferred embodiment, the metal is present at a concentration of less than about 70 ppb, e.g., a concentration of about 60 ppb. Maximum concentrations intermediate to the above recited concentrations, e.g., less than about 65 ppb, are also intended to be part of this invention. Further, ranges of values using a combination of any of the above recited values as upper and/or lower limits, e.g., concentrations between about 50 ppb and about 70 ppb, are also intended to be included.

In a preferred embodiment, the formulations of the invention are substantially free of metal following subjection to at least one procedure that removes metal, such as filtration, buffer exchange, chromatography or resin exchange. Procedures useful to remove metal from formulations of the invention are known to one of skill in the art and are further described herein, e.g., in the Examples below. In another preferred embodiment, the formulations of the invention comprise a metal chelator, e.g., such that the molecule is not cleaved within the hinge region or is cleaved within the hinge region at a level which is less than the level of cleavage observed in the absence of the metal chelator. In the formulations of the invention, the metal chelator may be, for example, a siderophore, calixerenes, an aminopolycarboxylic acid, a hydroxyaminocarboxylic acid, an N-substituted glycine, a 2-(2-amino-2-oxoethyl)aminoethane sulfonic acid (BES), a bidentate, tridentate or hexadentate iron chelator, a copper chelator, and derivatives, analogues, and combinations thereof. In one embodiment, the metal chelator is desferrioxamine. Metal chelators useful in formulations of the invention are known to one of skill in the art, and non-exclusive examples are described below.

Particular siderophores useful in formulations of the invention include, but are not limited to, aerobactin, agrobactin, azotobactin, bacillibactin, N-(5-C3-L (5 aminopentyl)hydroxycarbamoyl)-propionamido)pentyl)-3(5-(N-hydroxyacetoamido)pentyl)carbamoyl)-proprionhydroxamic acid (deferoxamine, desferrioxamine or DFO or DEF), desferrithiocin, enterobactin, erythrobactin, ferrichrome, ferrioxamine B, ferrioxamine E, fluviabactin, fusarinine C, mycobactin, parabactin, pseudobactin, vibriobactin, vulnibactin, yersiniabactin, ornibactin, and derivatives, analogues, and combinations thereof.

Aminopolycarboxylic acids useful in formulations of the invention include, but are not limited to, nitriloacetic acid (NTA), trans-diaminocyclohexane tetraacetic acid (DCTA), diethylenetriamine pentaacetic acid (DTPA), N-2-acetamido-2-iminodiacetic acid (ADA), aspartic acid, bis(aminoethyl)glycolether N,N,N′N′-tetraacetic acid (EGTA), glutamic acid, and N,N′-bis(2-hydroxybenzyl)ethylenediamine-N,N′-diacetic acid (HBED), and derivatives, analogues, and combinations thereof.

Hydroxyaminocarboxylic acids useful in formulations of the invention include, but are not limited to, N-hydroxyethyliminodiacetic acid (HIMDA), N,N-bishydroxyethylglycine (bicine), and N-(trishydroxymethylmethyl)glycine (tricine), and derivatives, analogues, and combinations thereof. N-substituted glycines, e.g., glycylglycine, as well as derivatives, analogues, or combinations thereof, are also useful as metal chelators in formulations of the invention. The metal chelator 2-(2-amino-2-oxoethyl)aminoethane sulfonic acid (BES), and derivatives, analogues, and combinations thereof, can also be used.

Particular calixarenes useful in formulations of the invention include, but are not limited to, a macrocycle or cyclic oligomer based on a hydroxyalkylation product of a phenol and an aldehyde, and derivatives, analogues, and combinations thereof. Particular copper chelators useful in the invention include triethylenetetramine (trientine), etraethylenepentamine, D-penicillamine, ethylenediamine, bispyridine, phenantroline, bathophenanthroline, neocuproine, bathocuproine sulphonate, cuprizone, cis,cis-1,3,5,-triaminocyclohexane (TACH), tachpyr, and derivatives, analogues, and combinations thereof.

Additional metal chelators that can be employed in formulations of the invention include citrate, a hydroxypyridine-derivate, a hydrazone-derivate, and hydroxyphenyl-derivate, or a nicotinyl-derivate, such as 1,2-dimethyl-3-hydroxypyridin-4-one (Deferiprone, DFP or Ferriprox); 2-deoxy-2-(N-carbamoylmethyl-[N′-2′-methyl-3′-hydroxypyridin-4′-one])-D-glucopyranose (Feralex-G), pyridoxal isonicotinyl hydrazone (P1H); 4,5-dihydro-2-(2,4-dihydroxyphenyl)-4-methylthiazole-4-carboxylic acid (GT56-252), 4-[3,5-bis(2-hydroxyphenyl)-[1,2,4]triazol-1-yl]benzoic acid (ICL-670); N,N′-bis(o-hydroxybenzyl)ethylenediamine-N,N′-diacetic acid (HBED), 5-chloro-7-iodo-quinolin-8-ol (clioquinol), and derivatives, analogues, and combinations thereof.

It will be recognized that combinations of two or more of any of the foregoing metal chelators can be used in combination in the formulations of the invention. For example, in a particular embodiment of the invention, the formulation comprises a combination of DTPA and DEF. In another embodiment, the formulation comprises a combination of EGTA and DEF.

In a preferred aspect, the formulations of the invention comprise a high protein concentration, including, for example, a protein concentration greater than about 45 mg/ml, a protein concentration greater than about 50 mg/ml, a protein concentration greater than about 100 mg/ml, a protein concentration greater than about 110 mg/ml, a protein concentration greater than about 120 mg/ml, a protein concentration greater than about 130 mg/ml, a protein concentration greater than about 140 mg/ml, a protein concentration greater than about 150 mg/ml, a protein concentration greater than about 160 mg/ml, a protein concentration greater than about 170 mg/ml, a protein concentration greater than about 180 mg/ml, a protein concentration greater than about 190 mg/ml, a protein concentration greater than about 200 mg/ml, a protein concentration greater than about 210 mg/ml, a protein concentration greater than about 220 mg/ml, a protein concentration greater than about 230 mg/ml, a protein concentration greater than about 240 mg/ml, a protein concentration greater than about 250 mg/ml, or a protein concentration greater than about 300 mg/ml. In a preferred embodiment of the invention, the protein comprises at least a portion of a lambda light chain. In a preferred embodiment of the invention, the protein is an antibody, e.g., an antibody comprising at least a portion of a lambda light chain. In a preferred embodiment of the invention, the antibody binds to the p40 subunit of Il-12/IL-23. In another preferred embodiment, the antibody is J695, e.g., as described in U.S. Pat. No. 6,914,128, the entire contents of which are incorporated by reference herein.

Preparation of the Antibody of Interest is Performed According to Standard methods known in the art. In a preferred embodiment of the invention, the antibody used in the formulation is expressed in a cell, such as, for example, a CHO cell, and purified by a standard series of chromatography steps. In a further preferred embodiment, the antibody is directed to the p40 subunit of IL-12/IL-23, and is prepared according to the methods described in U.S. Pat. No. 6,914,128, the entire contents of which are incorporated by reference herein.

After preparation of the antibody of interest, the pharmaceutical formulation comprising the antibody is prepared. The therapeutically effective amount of antibody present in the formulation is determined, for example, by taking into account the desired dose volumes and mode(s) of administration. In one embodiment of the invention, the concentration of the antibody in the formulation is between about 0.1 to about 250 mg of antibody per ml of liquid formulation. In one embodiment of the invention, the concentration of the antibody in the formulation is between about 1 to about 200 mg of antibody per ml of liquid formulation. In various embodiments, the concentration of the antibody in the formulation is between about 30 to about 140 mg per ml, between about 40 to about 120 mg/ml, between about 50 to about 110 mg/ml, or between about 60 to about 100 mg/ml. The formulation is especially suitable for large antibody dosages of more than 15 mg/ml. In various embodiments, the concentration of the antibody in the formulation is about 1, 10, 20, 30, 40, 50, 60, 70, 80, 90, 100, 110, 120, 130, 140, 150, 160, 170, 180, 190, 200, 210, 220, 230, 240 or 250 mg/ml. In a preferred embodiment, the concentration of the antibody is 50 mg/ml. In another preferred embodiment, the concentration of the antibody is 100 mg/ml. In a preferred embodiment, the concentration of the antibody is at least about 100 mg/ml, at least about 110 mg/ml or at least about 120 mg/ml.

In various embodiments of the invention, the concentration of the antibody in the formulation is about 0.1-250 mg/ml, 0.5-220 mg/ml, 1-210 mg/ml, about 5-200 mg/ml, about 10-195 mg/ml, about 15-190 mg/ml, about 20-185 mg/ml, about 25-180 mg/ml, about 30-175 mg/ml, about 35-170 mg/ml, about 40-165 mg/ml, about 45-160 mg/ml, about 50-155 mg/ml, about 55-150 mg/ml, about 60-145 mg/ml, about 65-140 mg/ml, about 70-135 mg/ml, about 75-130 mg/ml, about 80-125 mg/ml, about 85-120 mg/ml, about 90-H5 mg/ml, about 95-110 mg/ml, about 95-105 mg/ml, or about 100 mg/ml. Ranges intermediate to the above recited concentrations, e.g., about 31-174 mg/ml, are also intended to be part of this invention. For example, ranges of values using a combination of any of the above recited values as upper and/or lower limits are intended to be included.

In one embodiment, the invention provides a formulation with improved stability or an extended shelf life comprising of an active ingredient, preferably an antibody, in combination with a polyol, a surfactant and a buffer system with a pH of about 5 to 7. In one embodiment, the formulation further comprises a stabilizer. In one embodiment said formulation is free of metal. In a preferred embodiment, the formulation with improved stability of an extended shelf life comprises an active ingredient, preferably an antibody, and mannitol, histidine, methionine, polysorbate 80, hydrochloric acid, and water. In a further embodiment, the formulation of the invention has an extended shelf life of at least about 24 months at between about 2 and 8° C. in the liquid state. Freezing the formulation of the invention can also be used to further extend its shelf life. In a further embodiment, the formulation of the invention maintains stability following at least 5 freeze/thaw cycles of the formulation.

An aqueous formulation is prepared comprising the antibody in a pH-buffered solution. The buffer of this invention has a pH ranging from about 4 to about 8, preferably from about 4.5 to about 7.5, more preferably from about 5 to about 7, more preferably from about 5.5 to about 6.5, and most preferably has a pH of about 6.0 to about 6.2. In a particularly preferred embodiment, the buffer has a pH of about 6. In another preferred embodiment, the buffer has a pH of about 5 or less such as, for example, 2.5 to 5.0; 3.0 to 5.0, 3.5 to 5.0, 4.0 to 5.0, and 4.5 to 5.0. Ranges intermediate to the above recited pH's are also intended to be part of this invention. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate, phosphate, imidazole and other organic acid buffers. In a preferred embodiment of the invention, the formulation contains a buffer system comprising histidine. In a preferred embodiment of the invention, the buffer is histidine, e.g., L-histidine. In preferred embodiments, the formulation of the invention comprises a buffer system comprising about 1-100 mM histidine, preferably about 5-50 mM histidine, and most preferably 10 mM histidine. In another embodiment, the formulation comprises a buffer system comprising histidine and citrate or a buffer system comprising histidine and phosphate. In yet another embodiment, the formulation comprises a buffer system comprising imidazole. In yet another embodiment, the formulation comprises a buffer system comprising citrate and phosphate. One of skill in the art will recognize that sodium chloride can be used to modify the toxicity of the solution, e.g., at a concentration of 1-300 mM, and optimally 150 mM for a liquid dosage form.

A polyol, which acts as a tonicifier and may stabilize the antibody, is also included in the formulation. The polyol is added to the formulation in an amount that may vary with respect to the desired isotonicity of the formulation. Preferably the aqueous formulation is isotonic. The amount of polyol added may also vary with respect to the molecular weight of the polyol. For example, a lower amount of a monosaccharide (e.g., mannitol) may be added, compared to a disaccharide (such as trehalose). In a preferred embodiment of the invention, the polyol that is used in the formulation as a tonicity agent is mannitol. In a preferred embodiment, the composition comprises about 10 to about 100 mg/ml, or about 20 to about 80, about 20 to about 70, about 30 to about 60, about 30 to about 50 mg/ml of mannitol, for example, about 10, about 20, about 30, about 40, about 50, about 60, about 70, about 80, about 90, and about 100 mg/ml of mannitol In a preferred embodiment, the formulation comprises about 40 mg/ml of mannitol (corresponding to about 4% mannitol). In a preferred embodiment, the composition comprises between about 1% to about 10% mannitol, more preferably between about 2% to about 6% mannitol, and most preferably about 4% mannitol. In another embodiment of the invention, the polyol sorbitol is included in the formulation.

A stabilizer or antioxidant may also be added to the antibody formulations described herein. A stabilizer can be used in both liquid and lyophilized dosage forms. Formulations of the invention may comprise methionine, e.g., L-Methionine, as a stabilizer. For example, by getting oxidized, methionine may act to strengthen the stabilizing effect of the other buffers present in the formulation. However, in certain embodiments of the invention, under certain circumstances methionine is present in the formulations as part of the buffer system and not as a stabilizer, for example, methionine may be present in a formulation in an amount insufficient for acting as a stabilizer. Other stabilizers useful in formulations of the invention are known to those of skill in the art and include, but are not limited to, glycine and arginine. Cryoprotectants can be included for a lyophilized dosage form, principally sucrose (e.g., 1-10% sucrose, and optimally 0.5-1.0% sucrose). Other suitable cyroprotectants include trehalose and lactose.

A detergent or surfactant is also added to the antibody formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In a preferred embodiment of the invention, the formulation includes a surfactant that is a polysorbate. In another preferred embodiment of the invention, the formulation contains the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In one preferred embodiment, the formulation contains between 0.001 to about 0.1% polysorbate 80, or between about 0.005 and 0.05%, 20 polysorbate 80, for example, about 0.001, about 0.005, about 0.01, about 0.05, or about 0.1% polysorbate 80. In a preferred embodiment, about 0.01% polysorbate 80 is found in the formulation of the invention.

As described in the Examples herein, certain of the formulation components may be included or present in the formulation without negatively affecting the stability of the antibody molecule, e.g., without promoting or increasing fragmentation of the antibody molecule. For example, surfactants, e.g., polysorbates (e.g., polysorbate 80) or poloxamers (e.g., poloxamer 188), may be added to the formulation without promoting or increasing antibody fragmentation. Polyols, e.g., mannitol, may be added to the formulation without promoting or increasing antibody fragmentation. Amino acids, e.g., arginine, may also be added to the formulation without promoting or increasing antibody fragmentation. Organic based buffers, e.g., acetate, may be added to the formulation without promoting or increasing antibody fragmentation. Thus, acetate (acetic acid) may be used, for example, to lower the pH of the formulation without negatively affecting the stability of the antibody molecule. Further, salts, such as, e.g., NaCl, may be added to the formulation, since the ionic strength of the formulation has no effect on the stability, e.g., fragmentation, of the antibody molecule.

In a preferred embodiment of the invention, the formulation is a 1.0 mL solution in a container containing the ingredients shown below in Table 1. In another embodiment, the formulation is a 0.8 mL solution in a container.

TABLE 1
A 1.0 mL Solution¹⁾ of J695 Formulation for Injection

| Name of Ingredient | Quantity | | Function |
|---|---|---|---|
| Active substance: | | | |
| Antibody (J695)²⁾ | 50.0 or 100.0 | mg | Active substance |
| Excipients: | | | |
| Mannitol | 40 | mg | Tonicity agent |
| Polysorbate 80 | 0.10 | mg | Detergent/Surfactant |
| Histidine | 1.55 | mg | Buffer |
| Methionine | 1.49 | mg | Buffer |
| Water for injection | To one 1 | ml | Solvent |

| | | |
|---|---|---|
| Hydrochloric Acid | q.s. | pH adjustment to 6.0 |

¹⁾Density of the solution: 1.0398 g/mL
²⁾Is used as concentrate

In one embodiment, the formulation contains the above-identified agents (i.e., antibody, polyol/tonicity agent, surfactant and buffer) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. One or more other pharmaceutically acceptable carriers, excipients or stabilizers such as those described in Remington's Pharmaceutical Sciences 16th edition, Osol, A. Ed. (1980) may be included in the formulation provided that they do not significantly adversely affect the desired characteristics of the formulation. Acceptable carriers, excipients or stabilizers are nontoxic to recipients at the dosages and concentrations employed and include; additional buffering agents; co-solvents; antioxidants such as ascorbic acid; chelating agents such as EDTA; metal complexes (e.g. Zn-protein complexes); biodegradable polymers such as polyesters; and/or salt-forming counterions such as sodium.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection. Accordingly, preferably the antibody is prepared as an injectable solution. The injectable solution can be composed of either a liquid or lyophilized dosage form in a flint or amber vial, ampule or pre-filled syringe. In a preferred embodiment of the invention, the stable formulation comprising an antibody is prepared in a pre-filled syringe.

The formulation herein may also be combined with one or more other therapeutic agents as necessary for the particular indication being treated, preferably those with complementary activities that do not adversely affect the antibody of the formulation. Such therapeutic agents are suitably present in combination in amounts that are effective for the purpose intended. Such combination therapies may advantageously utilize lower dosages of the administered therapeutic agents (e.g., a synergistic therapeutic effect may be achieved through the use of combination therapy which, in turn, permits use of a lower dose of the antibody to achieve the desired therapeutic effect), thus avoiding possible toxicities or complications associated with the various monotherapies. In preferred embodiments of the invention, an antibody that binds the p40 subunit of II-12/IL-23 is coformulated with and/or coadministered with one or more additional therapeutic agents that are useful for treating disorders in which the activity of the p40 subunit of IL-12/IL-23 is detrimental. For example, an antibody or antibody portion of a formulation of the invention may be coformulated and/or coadministered with one or more additional antibodies that bind other targets (e.g., antibodies that bind other cytokines, e.g., IL-17, or that bind cell surface molecules). Furthermore, an antibody of a formulation of the invention may be used in combination with two or more of the foregoing therapeutic agents. Additional therapeutic agents which can be combined with the formulation of the invention are further described in U.S. Pat. No. 6,914,128, for example, at column 76, line 10 through column 78, line 53. The entire contents of U.S. Pat. No. 6,914,128 re incorporated herein by reference.

The formulations to be used for in vivo administration must be sterile. This is readily accomplished by filtration through sterile filtration membranes, prior to, or following, preparation of the formulation.

### V. Administration of Formulation

The formulation of the invention can be used in similar indications as those described in U.S. Pat. No. 6,914,128, the entire contents of which are incorporated by reference herein, and further detailed below.

In one aspect of the invention, the stable formulations of the invention comprise an antibody that binds to IL-12 and/or IL-23, e.g., binds to the p40 subunit of IL-12 and/or IL-23, and inhibits the activity of IL-12 and/or IL-23, e.g., inhibits the activity of the p40 subunit of IL-12 and/or IL-23. As used herein, the term “IL-12 and/or IL-23 activity-inhibiting formulation” is intended to include formulations comprising an antibody that binds to IL-12 and/or IL-23, e.g., binds to the p40 subunit of IL-12 and/or IL-23, and inhibits the activity of IL-12 and/or IL-23, e.g., inhibits the activity of the p40 subunit of IL-12 and/or IL-23.

The language “effective amount” of the formulation is that amount necessary or sufficient to inhibit IL-12 and/or IL-23 activity (e.g., to inhibit activity of the p40 subunit of IL-12/IL-23) e.g., prevent the various morphological and somatic symptoms of a detrimental IL-12 and/or IL-23 activity-associated state. In another embodiment, the effective amount of the formulation is the amount necessary to achieve the desired result. In one example, an effective amount of the formulation is the amount sufficient to inhibit detrimental IL-12 and/or IL-23 activity (e.g., detrimental activity of the p40 subunit of IL-12/IL-23). In another example, an effective amount of the formulation is 0.8 mL of the formulation containing 50 mg/ml or 100 mg/ml of antibody (e.g., 40 mg or 80 mg antibody), as described in Table 1. In another example, an effective amount of the formulation 1.0 mL of the formulation containing 50 mg/ml or 100 mg/ml of antibody (e.g., 50 mg or 100 mg antibody), as described in Table 1. The effective amount can vary depending on such factors as the size and weight of the subject, or the type of illness. For example, the choice of an IL-12 and/or IL-23 activity-inhibiting formulation can affect what constitutes an “effective amount”. One of ordinary skill in the art would be able to study the aforementioned factors and make the determination regarding the effective amount of the IL-12 and/or IL-23 activity inhibiting formulation without undue experimentation.

The regimen of administration can affect what constitutes an effective amount. The IL-12 and/or IL-23 activity-inhibiting formulation can be administered to the subject either prior to or after the onset of detrimental IL-12 and/or IL-23 activity. Further, several divided dosages, as well as staggered dosages, can be administered daily or sequentially, or the dose can be continuously infused, or can be a bolus injection. Further, the dosages of the IL-12 and/or IL-23 activity-inhibiting formulation can be proportionally increased or decreased as indicated by the exigencies of the therapeutic or prophylactic situation.

The term “treated,” “treating” or “treatment” includes the diminishment or alleviation of at least one symptom associated or caused by the state, disorder or disease being treated. For example, treatment can be diminishment of one or more symptoms of a disorder or complete eradication of a disorder.

Actual dosage levels of the active ingredients (antibody) in the pharmaceutical formulation of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the antibody found in the formulation, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition of the present invention required. For example, the physician or veterinarian could start doses of the compounds of the invention employed in the pharmaceutical formulation at levels lower than that required in order to achieve the desired therapeutic effect and gradually increase the dosage until the desired effect is achieved.

In general, a suitable daily dose of a formulation of the invention will be that amount of the formulation that is the lowest dose effective to produce a therapeutic effect. Such an effective dose will generally depend upon the factors described above. An effective amount of the formulation of the present invention is an amount that inhibits IL-12 and/or IL-23 activity (e.g., activity of the p40 subunit of IL-12/IL-23) in a subject suffering from a disorder in which IL-12 and/or IL-23 activity is detrimental. In a preferred embodiment, the formulation provides an effective dose of 40 mg, 50 mg, 80 or 100 mg per injection of the active ingredient, the antibody. In another embodiment, the formulation provides an effective dose which ranges from about 0.1 to 250 mg of antibody. If desired, the effective daily dose of the pharmaceutical formulation may be administered as two, three, four, five, six or more sub-doses administered separately at appropriate intervals throughout the day, optionally, in unit dosage forms.

In an embodiment of the invention, the dosage of the antibody in the formulation is between about 1 to about 200 mg. In an embodiment, the dosage of the antibody in the formulation is between about 30 and about 140 mg, between about 40 and about 120 mg, between about 50 and about 110 mg, between about 60 and about 100 mg, or between about 70 and about 90 mg. In a further embodiment, the composition includes an antibody dosage, or antigen binding fragment thereof, that binds to IL-12 and/or IL-23 (e.g., binds to the p40 subunit of IL-12 and/or IL-23, for example J695) for example, at about 1, 10, 20, 30, 40, 50, 60, 70, 80, 90, 100, 110, 120, 130, 140, 150, 160, 170, 180, 190, 200, 210, 220, 230, 240 or 250 mg.

Ranges intermediate to the above recited dosages, e.g., about 2-139 mg, are also intended to be part of this invention. For example, ranges of values using a combination of any of the above recited values as upper and/or lower limits are intended to be included.

It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The invention provides a pharmaceutical formulation with an extended shelf life, which, in one embodiment, is used to inhibit IL-12 and/or IL-23 activity (e.g., activity of the p40 subunit of IL-12 and/or IL-23) in a subject suffering from a disorder in which IL-12 and/or IL-23 activity is detrimental, comprising administering to the subject an antibody or antibody portion of the invention such that IL-12 and/or IL-23 activity in the subject is inhibited. Preferably, the IL-12 and/or IL-23 are human IL-12 and/or IL-23 and the subject is a human subject. Alternatively, the subject can be a mammal expressing an IL-12 and/or IL-23 with which an antibody of the invention cross-reacts. Still further the subject can be a mammal into which has been introduced IL-12 and/or IL-23 (e.g., by administration of IL-12 and/or IL-23 or by expression of an IL-12 and/or IL-23 transgene). A formulation of the invention can be administered to a human subject for therapeutic purposes (discussed further below). In one embodiment of the invention, the liquid pharmaceutical formulation is easily administratable, which includes, for example, a formulation which is self-administered by the patient. In a preferred embodiment, the formulation of the invention is administered through sc injection, preferably single use. Moreover, a formulation of the invention can be administered to a non-human mammal expressing an IL-12 and/or IL-23 with which the antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

As used herein, the term “a disorder in which the activity of the p40 subunit of IL-12 and/or IL-23 is detrimental” or “a disorder in which IL/12 and/or IL-23 activity is detrimental” is intended to include diseases and other disorders in which the presence of IL-12 and/or IL-23, e.g., the p40 subunit thereof, in a subject suffering from the disorder has been shown to be or is suspected of being either responsible for the pathophysiology of the disorder or a factor that contributes to a worsening of the disorder. Accordingly, a disorder in which IL-12 and/or IL-23 activity is detrimental is a disorder in which inhibition of the activity of IL-12 and/or IL-23, e.g., inhibition of the activity of the p40 subunit of IL-12 and/or IL-23, is expected to alleviate the symptoms and/or progression of the disorder. Such disorders may be evidenced, for example, by an increase in the concentration of IL-12 and/or IL-23, e.g., an increase in the concentration of the p40 subunit of IL-12 and/or IL-23, in a biological fluid of a subject suffering from the disorder (e.g., an increase in the concentration of IL-12 and/or IL-23, for example, the concentration of the p40 subunit of IL-12 and/or IL-23, in serum, plasma, synovial fluid, etc. of the subject), which can be detected, for example, using an anti-p40 IL-12 and/or IL-23 antibody as described above.

There are numerous examples of disorders in which IL-12 and/or IL-23 activity, e.g., the activity of the p40 subunit of IL-12 and/or IL-23, is detrimental. Examples of such disorders are described in U.S. Application No. 60/126,603, incorporated by reference herein. Examples of disorders in which IL-12 and/or IL-23 activity, e.g., the activity of the p40 subunit of IL-12 and/or IL-23, is detrimental are also described in U.S. Pat. No. 6,914,128, e.g., at column 81, line 9 through column 82, line 59, the entire contents of which are incorporated by reference herein.

The use of the formulations of the invention comprising an antibody that binds to IL-12 and/or IL-23, e.g., the p40 subunit of 11-12 and/or IL-23, in the treatment of specific disorders is discussed further below:

### A. Rheumatoid Arthritis:

Interleukin-12 and Interleukin-23 have been implicated in playing a role in inflammatory diseases such as rheumatoid arthritis. Inducible IL-12p40 message has been detected in synovia from rheumatoid arthritis patients and IL-12 has been shown to be present in the synovial fluids from patients with rheumatoid arthritis (see e.g., Morita et al., (1998) Arthritis and Rheumatism 41: 306-314). IL-12 positive cells have been found to be present in the sublining layer of the rheumatoid arthritis synovium. In the collagen induced arthritis (CIA) murine model for rheumatoid arthritis, treatment of mice with an anti-IL-12 mAb (rat anti-mouse IL-12 monoclonal antibody, C17.15) prior to arthritis profoundly suppressed the onset, and reduced the incidence and severity of disease. Treatment with the anti-IL-12 mAb early after onset of arthritis reduced severity, but later treatment of the mice with the anti-IL-12 mAb after the onset of disease had minimal effect on disease severity. Using gene-targeted mice lacking the p19 subunit of IL-23 or the p40 subunit of IL-12/23, IL-23 was shown to be critical for the development of collagen induced arthritis (Murphy et al. (2003) J. Exp. Med. 198(12):1951-1957).

Accordingly, the human antibodies, and antibody portions of the invention can be used to treat, for example, rheumatoid arthritis, juvenile rheumatoid arthritis, Lyme arthritis, rheumatoid spondylitis, osteoarthritis and gouty arthritis. Typically, the antibody, or antibody portion, is administered systemically, although for certain disorders, local administration of the antibody or antibody portion may be beneficial. An antibody, or antibody portion, of the invention also can be administered with one or more additional therapeutic agents useful in the treatment of autoimmune diseases.

### B. Crohn's Disease

Interleukin-12 and Interleukin-23 also play a role in inflammatory bowel disease, e.g., Crohn's disease and ulcerative colitis. Increased expression of IFN-γ and IL-12 occurs in the intestinal mucosa of patients with Crohn's disease (see e.g., Fais et al., (1994) J. Interferon Res. 14: 235-238; Parronchi et al., (1997) Amer. J. Pathol. 150: 823-832; Monteleone et al., (1997) Gastroenterology 112: 1169-1178; Berrebi et al., (1998) Amer. J Pathol. 152: 667-672). Anti-IL-12 antibodies have been shown to suppress disease in mouse models of colitis, e.g., TNBS induced colitis IL-2 knockout mice, and recently in IL-10 knock-out mice. Increased expression of IL-23 has also been observed in patients with Crohn's disease and in mouse models of inflammatory bowel disease, e.g., TNBS induced colitis and in RAG1 knockout mice. 11-23 has been shown to be essential for T cell-mediated colitis and to promote inflammation through IL-17- and IL-6-dependent mechanisms in mouse models of colitis, e.g., in IL-10 knockout mice (see e.g., review by Zhang et al., (2007) Intern. Immunopharmacology 7:409-416). Accordingly, the antibodies, and antibody portions, of the invention, can be used in the treatment of inflammatory bowel diseases.

### C. Multiple Sclerosis

Interleukin-12 and Interleukin-23 have been implicated as key mediators of multiple sclerosis. Expression of the inducible IL-12 p40 message or IL-12 itself can be demonstrated in lesions of patients with multiple sclerosis (Windhagen et al., (1995) J. Exp. Med. 182: 1985-1996, Drulovic et al., (1997) J. Neurol. Sci. 147: 145-150). Chronic progressive patients with multiple sclerosis have elevated circulating levels of IL-12. Investigations with T-cells and antigen presenting cells (APCs) from patients with multiple sclerosis revealed a self-perpetuating series of immune interactions as the basis of progressive multiple sclerosis leading to a Th1-type immune response. Increased secretion of IFN-γ from the T cells led to increased IL-12 production by APCs, which perpetuated the cycle leading to a chronic state of a Th1-type immune activation and disease (Balashov et al., (1997) Proc. Natl. Acad. Sci. 94: 599-603). The roles of IL-12 and IL-23 in multiple sclerosis have been investigated using mouse and rat experimental allergic encephalomyelitis (EAE) models of multiple sclerosis. In a relapsing-remitting EAE model of multiple sclerosis in mice, pretreatment with anti-IL-12 mAb delayed paralysis and reduced clinical scores, and treatment with anti-IL-12 mAb at the peak of paralysis or during the subsequent remission period reduced clinical scores. Also in the EAE mouse model, treatment with an antibody against the p19 subunit of IL-23 prevented induction of EAE and reversed established disease (Chen et al. 2006 J Clinical Investigation 116(5):1317-1326). Using gene-targeted mice lacking IL-23, IL-23 was shown to be critical for autoimmune inflammation of the brain (Cua et al. (2003) Nature 421:7440748). Antibodies against the p40 subunit of IL-12/IL-23 were shown to have beneficial activities in a nonhuman primate model of Multiple Sclerosis, e.g., EAE in the common marmoset (Hart et al. 2008 Neurodegenerative Dis. 5:38-52). (See also reviews by: Gran et al., 2004 Crit. Rev. Immunol. 24:111-128; McKenzie et al. 2006 Trends Immunol 27:17-23). Accordingly, the antibodies or antigen binding portions thereof of the invention may serve to alleviate symptoms associated with multiple sclerosis in humans.

### D. Insulin-Dependent Diabetes Mellitus

Interleukin-12 has been implicated as an important mediator of insulin-dependent diabetes mellitus (IDDM). IDDM was induced in NOD mice by administration of IL-12, and anti-IL-12 antibodies were protective in an adoptive transfer model of IDDM. Early onset IDDM patients often experience a so-called “honeymoon period” during which some residual islet cell function is maintained. These residual islet cells produce insulin and regulate blood glucose levels better than administered insulin. Treatment of these early onset patients with an anti-IL-12 antibody may prevent further destruction of islet cells, thereby maintaining an endogenous source of insulin. IL-23 has been implicated in exacerbating diabetes, based on the observation that IL-23 induced diabetes in mice if co-administered with sub diabetogenic multiple low doses of streptozotocin (see, e.g., review by Cooke 2006 Rev. Diabet. Stud. 3(2):72-75). Accordingly, the antibodies or antigen binding portions thereof of the invention may serve to alleviate symptoms associated with diabetes.

### E. Psoriasis

Interleukin-12 and Interleukin-23 have been implicated as key mediators in psoriasis. Psoriasis involves acute and chronic skin lesions that are associated with a TH1-type cytokine expression profile. (Hamid et al. (1996) J. Allergy Clin. Immunol. 1:225-231; Turka et al. (1995) Mol. Med. 1:690-699). In mice, both overexpression of the p40 subunit of IL-12/IL-23 and injection of recombinant IL-23 result in inflammatory skin disease, and administration of anti-IL-12 p40 antibodies to murine psoriasis models resolved the psoriatic lesions. IL-12 p35 and p40 mRNAs were detected in diseased human skin samples. In other studies, increased expression of both the p40 subunit of IL-12/IL-23 and the p19 subunit of IL-23 was observed in human psoriatic lesions, and decreased expression of IL-12 and IL-23 was observed after psoriasis therapy. A genetic polymorphism in the p40 subunit of IL-12 has been linked to increased susceptibility to psoriasis. (See, e.g., reviews by Torti et al. (2007) J. Am. Acad. Dermatol. 57(6):1059-1068; Fitch et al. (2007) Current Rheumatology Reports 9:461-467). IL-12 and IL-23 have also been identified as critical factors in psoriatic arthritis (see e.g., review by Hueber et al. 2007 Immunology Letters 114:59-65). Accordingly, the antibodies or antigen binding portions thereof of the invention may serve to alleviate chronic skin disorders such psoriasis, as well as psoriatic arthritis.

### F. Other Disorders

Interleukin 12 and/or Interleukin 23 play a critical role in the pathology associated with a variety of diseases involving immune and inflammatory elements. These diseases include, but are not limited to, rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. The human antibodies, and antibody portions of the invention can be used to treat autoimmune diseases, in particular those associated with inflammation, including, rheumatoid spondylitis, allergy, autoimmune diabetes, autoimmune uveitis.

Practice of the invention will be still more fully understood from the following examples, which are presented herein for illustration only and should not be construed as limiting the invention in any way.

The contents of all cited references (including literature references, patents, patent applications, and websites) that may be cited throughout this application are hereby expressly incorporated by reference. The practice of the invention will employ, unless otherwise indicated, conventional techniques of protein analysis, which are well known in the art.

### EXEMPLIFICATION

Example 1 provides methods and materials used in the performance of the invention, for example, as used in Examples 2-6. Example 2 describes the preparation of an exemplary liquid J695 antibody formulation. Example 3 provides experiments that demonstrate the stability of the liquid J695 formulation during repeated freeze/thaw cycles between −80° C. and 25° C. Example 4 provides experiments that demonstrate the stability of the liquid J695 formulation during long-term storage at various temperatures in the frozen state. Example 5 provides experiments that demonstrate the stability of the liquid J695 formulation during repeated freeze/thaw cycles between −80° C. and 37° C. Example 6 provides experiments that demonstrate the stability of the liquid J695 formulation during accelerated and long-term storage at various temperatures. Example 7 provides methods and materials used in performance of the invention, for example, as used in Examples 8-9. Example 8 provides demonstrates the cleavage of antibody containing lambda light chain in the presence of histidine and metal, e.g., copper or iron. Example 9 demonstrates antibody fragmentation and prevention thereof with regard to various parameters of antibody formulation and solution components. These parameters include, but are not limited to, solution pH, antibody concentration, ionic strength of the formulation, type and concentration of formulation buffer, surfactants, and stabilizing excipients. Example 10 shows fragmentation of J695 (100 and 2 mg/mL) at various levels of iron and at different temperatures.

### Example 1

### Analytical Methods Used to Monitor J695 Stability

### Example 1.1

### Cation Exchange HPLC

Cation Exchange HPLC was used to determine the identity and purity of the J695 drug substance using weak cation exchange high performance liquid chromatography (Shimadzu 10AD HPLC with SPD UV/VIS Detector or equivalent). Species were resolved on a weak cation-exchange stationary phase (Dionex ProPac WCX-10, 4 mm×250 mm, Dionex Corporation, Sunnyvale, Calif.) on the basis of charge. One hundred microliters, at a concentration of 1 mg/mL, were injected and the sample components were resolved utilizing increasing salt (sodium chloride) and decreasing pH gradient in a phosphate buffer system, (Mobile Phase A: 10 mM sodium phosphate dibasic, pH 7.5; Mobile Phase B: 20 mM sodium phosphate dibasic, 20 mM sodium acetate, 400 mM sodium chloride, pH 5.0) at a flow rate of 1.0 mL/min. Column temperature was maintained at 25° C. throughout the analysis and samples were maintained at 2-8° C. prior to being injected. Identity of peaks was determined by comparing the relative retention time of the main peak of interest (detected via absorbanse at 280 nm) for a sample against the reference standard material. The heterogeneity profile for the test sample chromatogram was compared to the reference standard chromatographic profile. The sum of the peak areas in the main isoform region, the acidic region and the basic region of the sample were each reported. All reagents were purchased from JT Baker, (Phillipsburg N.J.) unless stated differently.

### Example 1.2

### J695 Binding ELISA

The Binding ELISA was used to measure the relative binding capacity of the anti-IL-12 antibody J695 sample to IL-12 relative to that of reference standard. In this assay, rhIL-12 protein (ABC) was bound, through an overnight incubation at 2-8° C., to a 96 well microtiter plate (VWR International, West Chester, Pa.). Standard and samples were diluted serially in 50% 1×PBS with 50% Superblock blocking buffer (Pierce Biotechnology Inc, Rockford, Ill.) in PBS and 0.05% Surfactamp-20 (Pierce Biotechnology Inc, Rockford, Ill.), from 160 ng/mL to 0.625 ng/mL and loaded into the rhIL-12 coated wells of the 96 well microtiter plate. The captured J695 was then recognized with goat anti-human IgG-HRP (Pierce Biotechnology Inc, Rockford, Ill.). A TMB Substrate kit (Pierce Biotechnology Inc, Rockford, Ill.) was used as the substrate for a colorimetric readout. The percent relative binding capacity was calculated as the ratio of the “C” values from the 4-parameter curve fit for the standard and sample.

### Example 1.3

### Size Exclusion HPLC

Size Exclusion HPLC was used to determine the purity of J695 (Shimadzu 10AD HPLC with SPD UV/VIS Detector or equivalent). Ten microliters of a 2.0 mg/mL protein solution (maintained at 2-8° C.) were injected on the column to obtain sufficient signal for analysis. Species were separated isocratically at a flow rate of 0.75 mL per minute using a Superdex gel filtration column (GE Healthcare Bio-Sciences Corp, Piscataway, N.J.) or comparable stationary phase and 211 mM Na₂SO₄/92 mM Na₂HPO₄, pH 7.0 for the mobile phase. The column temperature was maintained at ambient temperature during the analysis. Test samples were injected in duplicate and monomeric J695 and other species were detected by absorbance at 214 nm. Purity was determined by comparing the area of J695 antibody to the total area of 214 nm absorbing components in the sample, excluding buffer-related peaks. The method was capable of resolving high molecular weight aggregates and antibody fragments from intact J695.

### Example 1.4

### Colloidal Blue-Stained Reducing and Non-Reducing SDS PAGE Gels

Colloidal Blue Stained Reducing and Non-reducing SDS PAGE gels were used to determine the purity of J695. Samples were prepared under reducing and non-reducing conditions by using sample buffer (2×tris-glycine SDS, Invitrogen Corp. Carlsbad, Calif.) with or without added mercaptoethanol, respectively. The samples and standards were initially in diluted in MilliQ water to 0.4 mg/mL and 0.1 mg/mL for reduced and non-reduced gels, respectively. Samples were diluted 1:1 with sample buffer and heated at approximately 60° C. for about 30 minutes with SDS, which binds and denatures proteins. The amount of SDS that binds to the protein was directly proportional to its molecular size. Molecular weight markers (Mark 12, unstained MW Markers, Invitrogen Corp. Carlsbad, Calif.), the test sample and standard (reduced and nonreduced) were loaded onto separate lanes of 12% (reduced) and 8-16% (non-reduced) tris-glycine commercial gels (Invitrogen Corp. Carlsbad, Calif.). Separation of protein species was completed in 1×tris-glycine running buffer using constant voltage of 60V for the first 30 minutes, and then 125V until the dye front has reached the bottom of the gel. Protein was detected with colloidal blue stain (Invitrogen Corp. Carlsbad, Calif.). A qualitative assessment of purity was achieved by comparison of the purity profile in the non-reduced gel to that of the test sample to the J695 reference standard. Scanning densitometry (UMAX scanner with Phoretix 1D densitometry software or equivalent) was used to determine the percent purity of the sample from the sum of the heavy and light chain detected on the gel run under reducing conditions.

### Example 1.5

### Protein Concentration (A₂₈₀)

Spectrophotometric measurement measured the protein concentration of J695 drug substance. Samples were diluted in triplicate to obtain an OD value at A₂₈₀ between 0.3 and 1.5 AU. Dilutions were prepared, in water, gravimetrically (by weight) using a Mettler Toledo Analytical balance. The spectrophotometer (Beckman DU800 or equivalent) was blanked at 280 nm. The absorbance of each sample and control was read at 280 nm, with the resulting values corrected for dilution and divided by the extinction coefficient to arrive at a protein concentration. For J695, the extinction coefficient value in AU/mg/mL was 1.42.

### Example 1.6

### J695 Bioassay

A J695 cell based bioassay measured the relative activity of J695 samples compared to a reference standard. NK-92 cells were stimulated with a defined concentration of IL-12 and mixed with variable concentrations of the anti IL-12 antibody J695. During the incubation period, the NK-92 cells secreted interferon-gamma (IFN-γ) in proportion to the amount of IL-12 in solution. The amount of IFN-γ was quantified using a commercially available ELISA kit. Using nonlinear regression, the IC₅₀ values of the sample and the reference standard were calculated. The activity of the individual sample was expressed as a percentage of the activity (mean IC₅₀ value) of the reference standard.

### Example 2

### Preparation of the J695 Formulation

The pharmaceutical formulation was made according to the following protocol.

Materials that were used in the formulation included: mannitol, histidine, methionine, polysorbate 80, water for the injections and hydrochloric acid, which was used as a 10% solution to adjust the pH, and protein concentrate (i.e., antibody concentrate).

### Example 2.1

### Preparation of 10 L of Buffer (Equivalent to 10.133 kg-Density of the Solution: 1.0133 g/mL)

Ingredients were weighed out as follows: 400.00 g mannitol, 15.50 g histidine, 14.90 g methionine, 1.00 g polysorbate 80, and 9.701 g of water for injection.

A 10% hydrochloric acid solution was prepared by combining 54.80 g of hydrochloric acid (37%) with 145.20 g of water for injection.

A buffer was prepared by dissolving the following pre-weighed ingredients (described above) in about 90% of the water for injection: mannitol, histidine, methionine, and polysorbate 80. The sequence of the addition of the buffer components did not impact buffer quality.

Following addition of all of the buffer constituents, the pH of the solution was adjusted to about pH 6 with the 10% hydrochloric acid and the final weight of the water was added.

### Example 2.2

### Preparation of 10 L of Formulation (Equivalent to 10.398 kg)

The buffer solution prepared in Example 2.1 was added to the thawed and, optionally, pooled antibody concentrate in the following manner: The J695 antibody concentrate was thawed in a water bath prior to the preparation of the pharmaceutical formulation. About 8.37 kg of antibody concentrate was used, which is equivalent to about 1.0 kg of protein with about 125 mg protein/mL protein concentrate. The density of the concentrate was about 1.0467 g/mL. The buffer was added while stirring, until the final weight of the bulk solution was reached.

The final formulation containing all of its ingredients was filtered through two sterile 0.22 μm membrane filters (hydrophilic polyvinylidene difluoride, 0.22 μm pore size) into a sterilized receptacle. The filtration medium used was filtration sterilized using nitrogen. Following sterilization, the formulation was packaged for use in either a vial or a pre-filled syringe.

The skilled artisan will appreciate that the weight quantities and/or weight-to-volume ratios recited herein, can be converted to moles and/or molarities using the art-recognized molecular weights of the recited ingredients. Weight quantities exemplified herein (e.g., g or kg) are for the volumes (e.g., of buffer or pharmaceutical formulation) recited. The skilled artisan will appreciate that the weight quantities can be proportionally adjusted when different formulation volumes are desired. For example, 32 L, 20 L, 5 L, or 1 L formulations would include 320%, 200%, 50% or 10%, respectively, of the exemplified weight quantities.

### Example 3

### Physico-Chemical Analysis Of Stabilized Liquid J695 Formulation During Repeated Freeze/Thaw Studies (−80° C./25° C.)

After the formulation buffer for the J695 antibody was selected, the drug substance was formulated in the same matrix as the finished product. The primary goal of protein formulation is to maintain the stability of a given protein in its native, pharmaceutically active form over prolonged periods of time to guarantee acceptable shelf-life of the pharmaceutical protein drug. Typically, long shelf-life is achieved by storing the protein in frozen from (e.g., at −80° C.) or by subjecting the protein to a lyophilization process, i.e., by storing the protein in lyophilized form, and reconstituting it immediately before use. However, it is well known to those skilled in the art that freezing and thawing processes often impact protein stability, meaning that even storage of the pharmaceutical protein in frozen form can be associated with the loss of stability due to the freezing and thawing step. Also, the first process step of lyophilization involves freezing, which can negatively impact protein stability. Since it is well known that the risk of encountering protein instability phenomena increases with increasing protein concentration, achieving formulation conditions that maintain protein stability at high protein concentrations is a challenging task.

The freeze thaw behavior of the J695 antibody at a protein concentration of 138 mg/mL was evaluated by cycling drug substance up to 5 times between the frozen state and the liquid state. Freezing was performed by means of a temperature controlled −80° C. freezer, and thawing was performed by means of a 25° C. temperature controlled water bath. About 30 mL of J695 solution each were filled in 30 mL PETG repositories for this experiment. Table 2 provides an overview on testing intervals and the number of freeze/thaw cycles performed. The criteria defining desirable quality and stability of J695 antibody for this study is listed in Table 3.

TABLE 2
Testing Intervals: Number Of Freeze (−80° C.) And Thaw
(25° C. Water Bath) Cycles Applied

Testing Intervals:
Number of Freeze/Thaw
Cycles and Sample
Requirements for Testing

| | | | | |
|---|---|---|---|---|
| storage | T₀ | 1 | 3 | 5 |
| temperature | | | | |
| for stress test | | | | |
| −80° C./25° C. | 2* | 1* | 1* | 2* |
| cycling study | | | | |

*Number defines the number of repositories pulled and tested

The criteria defining desirable quality and stability of J695 antibody for this study are the same as listed in Table 3.

TABLE 3
Parameters Defining Desirable Quality And Stability Of J695
Antibody For Various Stress Studies

| Test | Check Specifications |
|---|---|
| Clarity | ≦EP Reference Suspension IV |
| Color | ≦Reference Solution BY4 |
| pH | 5.5-6.5 |
| Activity | 70%-130% relative percent binding capacity |
| ELISA | |
| SEC HPLC | Purity: ≧98.0% Monomer |
| | ≦2% Aggregates |
| CEX-HPLC | The predominant chromatographic pattern |
| | Conforms to that of the reference material |
| | Sum of Major Isoforms ≧85% |
| | Sum of Acidic Region ≦15% |
| | Sum of Basic Region ≦10% |
| SDS-PAGE | The predominant banding pattern conforms to |
| Colloidal | that of the reference standard |
| Reduced | Purity: Sum of Heavy + Light Chain ≧97% |
| SDS-PAGE | The predominant banding pattern conforms to |
| Colloidal | that of the reference standard |
| Non-Reduced | |
| *Endotoxin | ≦0.2 EU/mg |
| *Bioburden | ≦1 CFU/mL |

*These tests were performed at the time zero and at the end of study.

Results of the experiment evaluating the effect of five freeze-thaw cycles where J695 is formulated at least 110 mg/mL at a pH of about 6 (6.2) are reported in Table 4. Table 4 shows that the J695 antibody can be subjected to repeated freeze/thaw cycles for at least five times without any detrimental effect on either chemical properties (cation exchange HPLC, size exclusion HPLC, color, pH), physicochemical properties (clarity, reduced and non reduced SDS PAGE) or biological activity (activity ELISA assay) when formulated in the pharmaceutical composition of the invention as described in Example 2.

TABLE 4
Test Results Of A Freeze/Thaw Study Of J695 Antibody Formulated At
138 mg/mL In The Formulation as described in Example 2

| | Stability parameter | | Number of freeze/thaw | |
|---|---|---|---|---|
| Test Criteria | compared | Shelf life Specification | cycles | Data |
| Color | < or = Reference | < or = EP Reference | Initial Testing | ≦ BY5 |
| | Solution BY6 | Solution B7 | | |
| | | | 1 | ≦ BY5 |
| | | | 3 | ≦ BY5 |
| | | | 5 | ≦ BY5 |
| Clarity | Report values relative | < or = EP Reference | Initial Testing | ≦11 |
| | to reference | Suspension IV | | |
| | solution/suspension | | 1 | ≦11 |
| | | | 3 | ≦11 |
| | | | 5 | ≦11 |
| pH | 5.5 to 6.5 | 5.0 to 5.4 | Initial Testing | 6.2 |
| | | | 1 | 6.2 |
| | | | 3 | 6.2 |
| | | | 5 | 6.2 |
| Activity | > or = 70% of | 65% to 130% | Initial Testing | 89 |
| ELISA | observed protein | | | |
| | concentration | | | |
| (QCA-260) | | | 1 | 93 |
| | | | 3 | 99 |
| | | | 5 | 101 |
| SEC HPLC | Purity: > or = 90.0% | Purity Monomer > or = | Initial Testing | 99.5 |
| | | 99% | | |
| | | | 1 | 99.4 |
| | | | 3 | 99.4 |
| | | | 5 | 99.4 |
| CEX-HPLC | The predominant | Lysine Variants > or = 80% | Initial Testing | Conforms |
| | chromatographic | | | |
| | pattern conforms to | | 1 | Conforms |
| | that of the reference | | | |
| | | | 3 | Conforms |
| | | | 5 | Conforms |
| CEX-HPLC | Peak 1 relative | 1st Acidic Region: < or = | Initial Testing | 1.00 |
| | retention time | 6% | | |
| | 0.95-1.05 | | 1 | 1.00 |
| | | | 3 | 1.00 |
| | | | 5 | 1.00 |
| SDS-PAGE | The predominant | N/A | Initial Testing | Conforms |
| | banding | | | |
| Colloidal | Pattern conforms to | | 1 | Conforms |
| Blue Stain | that of | | | |
| Reduced | the reference standard | | 3 | Conforms |
| | | | 5 | Conforms |
| SDS-PAGE | The predominant | N/A | Initial Testing | Conforms |
| | banding | | | |
| Colloidal | pattern conforms to | | 1 | Conforms |
| Blue Stain | that of | | | |
| Non-Reduced | the reference standard | | 3 | Conforms |
| | | | 5 | Conforms |
| Bioburden | < or = 1 CFU/mL | < or = 1 CFU/mL | Initial Testing | 0 |
| | | | 1 | NP |
| | | | 3 | NP |
| | | | 5 | 0 |
| Endotoxin | < or = 0.2 EU/mg | < or = 0.2 EU/mg | Initial Testing | <0.1 |
| | | | 1 | NP |
| | | | 3 | NP |
| | | | 5 | <0.1 |

### Example 4

### Physico-Chemical Analysis of Stabilized Liquid J695 Formulation During Long-Term Storage at Various Temperatures in the Frozen State

In order to accommodate shelf-life of final drug product as well as drug product manufacturing strategies, logistics and shipment of final drug product, the bulk protein (i.e., drug substance, active pharmaceutical ingredient, API) is formulated in a formulation that maintains stability of the pharmaceutical protein in the frozen state for longer time periods. Ideally, the protein formulation maintains stability at various temperatures in frozen state, e.g., at −80° C., −40° C., and −20° C., to accommodate flexibility of storage locations of the bulk protein between bulk protein manufacture and drug product fill-finish. Those skilled in the art will acknowledge that this is a very challenging task.

Storage stability of the J695 antibody at a protein concentration of 121 mg/mL was evaluated at various temperatures within a −20° C. and −80° C. range for prolonged periods of time at controlled temperature conditions. After defined storage periods, the bulk protein was thawed and the impact of storage time and storage temperature on J695 stability was evaluated. About 1600 mL of J695 solution each were filled in 2 L polyethylene terephthalate copolyester (PETG) repositories for this experiment. Table 4 provides an overview on testing intervals and respective storage temperatures of J695 antibody applied in this experiment.

Results of the experiment evaluating the effect of storage time and storage temperature where J695 is formulated at least 110 mg/mL at a pH of about 6 (6.2) are reported in Table 5.

TABLE 5
Testing Intervals: Storage Temperatures And Sample Pull
Points Applied During Stability Experiment

| Storage | | | | | | | | | |
|---|---|---|---|---|---|---|---|---|---|
| Temperature | Testing Intervals (Months) | | | | | | | | |
| (nominal) | 0 | 0.25 | 0.50 | 1 | 3 | 6 | 9 | 12 | 18 |
| −80° C. | 2** | 1 | 1 | 1 | 1 | 1 | 1 | 2 | 1 |
| −40° C.* | NP | NP | NP | NP | 1 | NP | 1 | NP | 1 |
| −35° C.* | NP | NP | NP | NP | 1 | NP | 1 | NP | 1 |
| −30° C.* | NP | NP | NP | NP | 1 | NP | 1 | NP | 1 |
| −25° C.* | NP | NP | NP | NP | 1 | NP | 1 | NP | 1 |
| −20° C.* | NP | NP | NP | NP | 1 | NP | 1 | NP | 1 |

*Number defines the number of repositories pulled and tested
NP = not performed

Table 6 demonstrates that the J695 antibody can be subjected to storage for at least 18 months at various temperatures within a −20° C. and −80° C. range without detrimental effect on physical and chemical stability. For instance, over a storage time of 18 months, J695 antibody samples exhibited monomer levels of at least 98% for all temperatures at which the frozen antibody solution was stored. Similarly, data of activity ELISA demonstrated that J695 antibody samples tested exhibited high activity, independent of the temperature at which the frozen J695 antibody solution was stored. With regard to chemical stability of J695 monitored by cation exchange HPLC, data demonstrated that chemical stability of J695 antibody is not impacted over at least 18 months when stored in frozen form at temperatures between −20° C. and −80° C. In summary, the data demonstrate that J695 antibody can be subjected to storage for at least 18 months at various temperatures within a −20° C. and −80° C. range without negative impact on either chemical properties (cation exchange HPLC, size exclusion HPLC, color, pH), physicochemical properties (clarity, reduced and non reduced SDS PAGE) or biological properties (activity ELISA assay, bioburden, endotoxin levels) when formulated in the pharmaceutical composition as described in Example 2

TABLE 6
Test Results Of A Stability Study Employing Various Storage Temperatures Of J695 Antibody Formulated At 121 mg/mL as
Described in Example 2

| | | | Duration of | Storage | Storage | Storage | Storage | Storage | Storage |
|---|---|---|---|---|---|---|---|---|---|
| | Check | Stability | Testing | Conditions | Conditions | Conditions | Conditions | Conditions | Conditions |
| Test Criteria | Specification | Criterion | (months) | −80° C. | −40° C. | −35° C. | −30° C. | −25° C. | −20° C. |
| Color | < or = Reference | < or = EP | Initial | | | | | | |
| | Solution BY6 | Reference | Testing | | | | | | |
| | | Solution B7 | 0.25 | < or = | NP | NP | NP | NP | NP |
| | | | | BY6 | | | | | |
| | | | 0.5 | < or = | NP | NP | NP | NP | NP |
| | | | | BY6 | | | | | |
| | | | 1 | < or = | NP | NP | NP | NP | NP |
| | | | | BY6 | | | | | |
| | | | 3 | < or = | < or = | < or = | < or = | < or = | < or = |
| | | | | BY6 | BY6 | BY6 | BY6 | BY6 | BY6 |
| | | | 6 | < or = | NP | NP | NP | NP | NP |
| | | | | BY6 | | | | | |
| | | | 9 | < or = | < or = | < or = | < or = | < or = | < or = |
| | | | | BY6 | BY6 | BY6 | BY6 | BY6 | BY6 |
| | | | 12 | < or = | NP | NP | NP | NP | NP |
| | | | | BY5 | | | | | |
| | | | 18 | < or = | < or = | < or = | < or = | < or = | < or = |
| | | | | BY5 | BY5 | BY5 | BY5 | BY5 | BY5 |
| Clarity | Report values | < or = EP | Initial | < or = II | | | | | |
| | relative to | Reference | Testing | | | | | | |
| | reference | suspension IV | 0.25 | < or = II | NP | NP | NP | NP | NP |
| | solution/ | | 0.5 | < or = II | NP | NP | NP | NP | NP |
| | suspension | | 1 | < or = II | NP | NP | NP | NP | NP |
| | | | 3 | < or = II | < or = II | < or = II | < or = II | < or = II | < or = II |
| | | | 6 | < or = II | NP | NP | NP | NP | NP |
| | | | 9 | < or = II | < or = II | < or = II | < or = II | < or = III | < or = III |
| | | | 12 | < or = II | NP | NP | NP | NP | NP |
| | | | 18 | < or = II | < or = II | < or = II | < or = III | < or = III | < or = III |
| pH | 5.5 to 6.5 | 5.0 to 6.4 | Initial | 6.3 | | | | | |
| | | | Testing | | | | | | |
| | | | 0.25 | 6.3 | NP | NP | NP | NP | NP |
| | | | 0.5 | 6.3 | NP | NP | NP | NP | NP |
| | | | 1 | 6.3 | NP | NP | NP | NP | NP |
| | | | 3 | 6.3 | 6.3 | 6.3 | 6.3 | 6.3 | 6.3 |
| | | | 6 | 6.3 | NP | NP | NP | NP | NP |
| | | | 9 | 6.3 | 6.3 | 6.3 | 6.3 | 6.3 | 6.3 |
| | | | 12 | 6.2 | NP | NP | NP | NP | NP |
| | | | 18 | 6.3 | 6.3 | 6.3 | 6.3 | 6.3 | 6.2 |
| Activity | > or 60% | 75% to 125% | Initial | 99 | | | | | |
| Elisa | Binding Activity | | Testing | | | | | | |
| | protein | | 0.25 | 96 | NP | NP | NP | NP | NP |
| | concentration | | 0.5 | 95 | NP | NP | NP | NP | NP |
| | | | 1 | 106 | NP | NP | NP | NP | NP |
| | | | 3 | 92 | 92 | 121 | 91 | 92 | 93 |
| | | | 6 | 87 | NP | NP | NP | NP | NP |
| | | | 9 | 99 | 100 | 110 | 102 | 98 | 105 |
| | | | 12 | 101 | NP | NP | NP | NP | NP |
| | | | 18 | 93 | 94 | 94 | 97 | 98 | 96 |
| SEC | Purity: > or | Purity | Initial | 99.4 | | | | | |
| HPLC | =90.0% | Monomer > | Testing | | | | | | |
| | | or =99% | 0.25 | 99.4 | NP | NP | NP | NP | NP |
| | | | 0.5 | 99.4 | NP | NP | NP | NP | NP |
| | | | 1 | 99.5 | NP | NP | NP | NP | NP |
| | | | 3 | 99.4 | 99.3 | 99.4 | 99.2 | 98.9 | 98.5 |
| | | | 6 | 99.4 | NP | NP | NP | NP | NP |
| | | | 9 | 99.4 | 99.4 | 99.4 | 99.4 | 98.2 | 99 |
| | | | 12 | 99.4 | NP | NP | NP | NP | NP |
| | | | 18 | 99.4 | 99.3 | 99.3 | 99.3 | 98.3 | 98.5 |
| CEX- | The predominant | Lysine Variants > or | Initial | Conforms | | | | | |
| HPLC | chromatographic | =80% | Testing | | | | | | |
| | pattern conforms to that of | | 0.25 | Conforms | NP | NP | NP | NP | NP |
| | the | | 0.5 | Conforms | NP | NP | NP | NP | NP |
| | reference standard | | 1 | Conforms | NP | NP | NP | NP | NP |
| | | | 3 | Conforms | Conforms | Conforms | Conforms | Conforms | Conforms |
| | | | 6 | Conforms | NP | NP | NP | NP | NP |
| | | | 9 | Conforms | Conforms | Conforms | Conforms | Conforms | Conforms |
| | | | 12 | Conforms | NP | NP | NP | NP | NP |
| | | | 18 | Conforms | Conforms | Conforms | Conforms | Conforms | Conforms |
| CEX- | Peak 1 relative retention | 1st Acidic Region: | Initial | 1.00 | | | | | |
| HPLC | time | < or =6% | Testing | | | | | | |
| | 0.98-1.05 | | 0.25 | 1.00 | NP | NP | NP | NP | NP |
| | | | 0.5 | 1.00 | NP | NP | NP | NP | NP |
| | | | 1 | 1.00 | NP | NP | NP | NP | NP |
| | | | 3 | 1.00 | 1.00 | 1.00 | 1.00 | 1.00 | 1.00 |
| | | | 6 | 1.00 | NP | NP | NP | NP | NP |
| | | | 9 | 1.00 | 1.00 | 1.00 | 1.00 | 1.00 | 1.00 |
| | | | 12 | 1.00 | NP | NP | NP | NP | NP |
| | | | 18 | 1.00 | 1.00 | 1.00 | 1.00 | 1.00 | 1.01 |
| SDS- | predominant banding | Clarity: > or =98% | Initial | Conforms | | | | | |
| PAGE | pattern | | Testing | | | | | | |
| Colloidal | conforms to that of the | | 0.25 | Conforms | NP | NP | NP | NP | NP |
| Blue Stain | reference standard | | 0.5 | Conforms | NP | NP | NP | NP | NP |
| Reduced | | | 1 | Conforms | NP | NP | NP | NP | NP |
| | | | 3 | Conforms | Conforms | Conforms | Conforms | Conforms | Conforms |
| | | | 6 | Conforms | NP | NP | NP | NP | NP |
| | | | 9 | Conforms | Conforms | Conforms | Conforms | Conforms | Conforms |
| | | | 12 | Conforms | NP | NP | NP | NP | NP |
| | | | 18 | Conforms | Conforms | Conforms | Conforms | Conforms | Conforms |
| SDS- | Predominant banding | >200 kDa Band(s): | Initial | Conforms | | | | | |
| PAGE | pattern | < or =3% | Testing | | | | | | |
| Colloidal | Conforms to that of the | | 0.25 | Conforms | NP | NP | NP | NP | NP |
| Blue Stain | reference standard | | 0.5 | Conforms | NP | NP | NP | NP | NP |
| Non- | | | 1 | Conforms | NP | NP | NP | NP | NP |
| Reduced | | | 3 | Conforms | Conforms | Conforms | Conforms | Conforms | Conforms |
| | | | 6 | Conforms | NP | NP | NP | NP | NP |
| | | | 9 | Conforms | Conforms | Conforms | Conforms | Conforms | Conforms |
| | | | 12 | Conforms | NP | NP | NP | NP | NP |
| | | | 18 | Conforms | Conforms | Conforms | Conforms | Conforms | Conforms |
| Bioburden | < or = 1 CFU/ml | | Initial | 0 | | | | | |
| | | | Testing | | | | | | |
| | | | 6 | NP | | | | | |
| | | | 12 | 0 | P | P | P | P | P |
| | | | 60 | P | P | P | P | P | P |
| Endotoxin | < or = 0.2 EU/mg | | Initial | <0.1 | | | | | |
| | | | Testing | | | | | | |
| | | | 6 | NP | | | | | |
| | | | 12 | <0.1 | P | P | P | P | P |
| | | | 60 | P | P | P | P | P | P |

### Example 5

### Physico-Chemical Analysis Of Stabilized Liquid J695 Formulation During Repeated Freeze/Thaw Studies (−80° C./37° C.)

After the formulation buffer for the J695 antibody was selected the drug substance was formulated in the same matrix as the finished product.

The freeze thaw behavior of the J695 antibody drug substance at a protein concentration of at least 100 mg/mL was evaluated by cycling two different drug substance batches (formulated as described in Example 2) five times from the frozen state to the liquid state. For this purpose 2 L PETG bottles were used containing approx. 1.6 L of J695 in the formulation as described in Example 2.

Table 7 shows the results of an experiment evaluating the effect of five freeze-thaw cycles in the formulation buffer starting from −80° C. The solutions were thawed within a water bath adjusted to 37° C. and were removed immediately after complete thawing for sample testing.

TABLE 7
Test Results Of A Freeze/Thaw Study Of J695 Antibody Formulated as
Described in Example 2*

| | Test criteria | | | | | | | | | | | |
|---|---|---|---|---|---|---|---|---|---|---|---|---|
| | | | 1 × | | 2 × | | 3 × | | 4 × | | 5 × | |
| | Initial | | Freeze/ | | Freeze/ | | Freeze/ | | Freeze/ | | Freeze/ | |
| | value | | Thawing | | Thawing | | Thawing | | Thawing | | Thawing | |
| | Batch No. | | | | | | | | | | | |
| | 1 | 2 | 1 | 2 | 1 | 2 | 1 | 2 | 1 | 2 | 1 | 2 |
| Clarity NTU | .07 | .72 | .14 | .78 | .03 | .82 | .64 | .63 | .78 | .89 | .56 | .71 |
| PCS Z-Average | .06 | .55 | .07 | .57 | .06 | .54 | .07 | .54 | .08 | .55 | .06 | .52 |
| [nm] | | | | | | | | | | | | |
| Subvisible | | | | | | | | | | | | |
| particles/1.0 mL | | | | | | | | | | | | |
| >1 μm | 44 | 9 | 39 | 06 | 4 | 7 | 2 | 6 | 6 | 2 | 8 | 07 |
| >10 μm | | | | | | | | | | | | |
| >25 μm | | | | | | | | | | | | |
| Size exclusion | | | | | | | | | | | | |
| HPLC | | | | | | | | | | | | |
| Aggregate [%] | .44 | .69 | .44 | .72 | .44 | .73 | .47 | .75 | .49 | .77 | .50 | .82 |
| Monomer [%] | 9.42 | 9.12 | 9.42 | 9.09 | 9.42 | 9.08 | 9.40 | 9.08 | 9.35 | 9.05 | 9.35 | 9.02 |
| Fragment [%] | .14 | .19 | .14 | .19 | .14 | .19 | .12 | .18 | .16 | .18 | .15 | .17 |

Table 7 shows that the J695 antibody drug substance in the formulation buffer can be freeze/thawed at least five times without any detrimental effect on physico-chemical properties, as monitored by clarity measurement, PCS, subvisible particle measurement and size exclusion HPLC.

For instance, over a series of five freeze/thaw cycles all J695 antibody samples tested exhibited monomer levels of at least 98%. Generally, freeze/thaw processing of antibody solutions is known for its high risks for inducing protein instability, which may be reflected increase in aggregate and elevated numbers of subvisible particles. When formulated in the pharmaceutical composition as described in Example 2, over a series of five freeze/thaw processing cycles virtually no change in aggregate levels (levels for all samples tested below 1%), no change in fragment levels (levels for all samples tested far below 0.5%), and no change in numbers of subvisible particles (data virtually unchanged throughout the whole freeze/thaw study) was monitored.

### Example 6

### Physico-Chemical Analysis Of Stabilized Liquid J695 Formulation During Accelerated And Long-Term Storage

Storage stability of the J695 antibody at a protein concentration of 100 mg/mL was evaluated at various temperatures for prolonged periods of time when J695 Drug Product was stored at controlled temperature conditions. After defined storage periods, samples were pulled and the impact of storage time and storage temperature on J695 stability was evaluated.

About 1 mL of J695 solution each were filled in 1 mL glass syringes for this experiment (primary packing: SFIF007A: SCF syringe, Becton Dickinson, combined with a Fluorotec piston stopper 4023/50). Table 8 provides an overview on testing intervals and respective storage temperatures of J695 antibody.

TABLE 8
Testing Intervals: Storage Temperatures And Sample Pull
Points Applied During Stability Experiment Of 100 mg/mL
J695 Drug Product

| | |
|---|---|
| Storage | Testing Interval in Months |

| Conditions | 0 | 1.5 | 3 | 6 | 12 | 24 |
|---|---|---|---|---|---|---|
| +5° C. | X | X | X | X | X | X |
| 25° C./60% RH | | X | X | X | — | — |
| 40° C./75% RH | | X | X | X | — | — |

X defines the time points at which J695 samples were pulled and analyzed.

The analytical tests used to assess the stability of the liquid drug product were either developed methods or pharmacopoeial methods. The methods were applied as described above for testing of J695 liquid drug product and were performed as described in the cited pharmacopoeia.

Results of the experiment evaluating the effect of storage time and storage temperature where J695 was formulated at 100 mg/mL at a pH of about 6 are reported in Table 9. Table 9 demonstrates that the J695 antibody can be subjected to storage for at least 24 months at a temperature range between 2° C. and 8° C. without detrimental effect on physical and chemical stability. For instance, over a storage time of 24 months, all J695 antibody samples tested remained virtually unchanged with regard to clarity, color, appearance, subvisible particle levels, and pH. Furthermore, over a period of at least 24 months, J695 formulated as described in Example 2 at 100 mg/mL exhibited monomer levels of at least 98%, with fragment levels being well below 0.5%. Even at accelerated storage conditions, J695 was highly stable, with monomer levels exceeding 90% even after storage at 40° C. for 6 months.

With regard to chemical stability, J695 antibody formulated in the composition as described in Example 2 at 100 mg/mL exhibited main isoform levels of at least 80% for at least 24 months at 2-8° C., with basic specimen levels being well below 10%, and acidic specimen levels being well below 20%. Even at accelerated storage conditions, J695 was highly stable, with main isoform levels exceeding 80%, basic specimen levels being well below 10% and acidic specimen levels being well below 20%, for all temperatures at which the frozen antibody solution was stored, even after storage at 25° C. for 6 months.

In summary, data demonstrate that J695 antibody can be subjected to storage for at least 24 months at 2 to 8° C. without negative impact on either chemical properties (cation exchange HPLC, size exclusion HPLC, color, pH), physicochemical properties (clarity, subvisible particle levels, size exclusion HPLC) or other properties (activity ELISA assay, protein concentration) when formulated in the pharmaceutical composition as described in Example 2.

TABLE 9
Test Results Of An Accelerated And Long-Term Stability Study Of J695
Antibody Formulated as Described in Example 2

| | | Duration | | | | |
|---|---|---|---|---|---|---|
| | | of testing | | Storage conditions [° C./% RH] | | |
| Test criteria | Quality Parameter | [months] | | +5 | +25/60 | 40/75 |
| Appearance | Colorless to slightly yellow | Initial | | Conforms | | |
| | solution | 1.5 | | Conforms | Conforms | Conforms |
| | | 3 | | Conforms | Conforms | Conforms |
| | | 6 | | Conforms | Conforms | Conforms |
| | | 12 | | Conforms | — | — |
| | | 24 | | Conforms | — | — |
| Clarity | Not more opalescent than | Initial | | <RS III | | |
| | reference suspension IV; ≦RS IV | 1.5 | | =RS II | <RS III | <RS III |
| | | 3 | | =RS II | <RS III | <RS III |
| | | 6 | | =RS II | =RS II | =RS III |
| | | 12 | | =RS II | — | — |
| | | 24 | | <RS II | — | — |
| Colour | Not more intensely colored than | Initial | | ≦BY6 | | |
| (visual comparison vs | reference solution BY4; ≦BY4 | 1.5 | | ≦BY6 | ≦BY6 | ≦BY6 |
| colour reference | | 3 | | ≦BY6 | ≦BY6 | ≦BY6 |
| solutions, EP) | | 6 | | ≦BY6 | ≦BY6 | ≦BY5 |
| | | 12 | | ≦BY6 | — | — |
| | | 24 | | ≦BY6 | — | — |
| PH | 5.5 to 6.5 | Initial | | 6.2 | | |
| | | 1.5 | | 6.1 | 6.1 | 6.1 |
| | | 3 | | 6.2 | 6.2 | 6.2 |
| | | 6 | | 6.2 | 6.2 | 6.2 |
| | | 12 | | 6.2 | — | — |
| | | 24 | | 6.2 | — | — |
| Particulate contamination | Report Result (Visual Score) | Initial | | 0 | | |
| Visible particles | | 1.5 | | 0.2 | 0 | 0 |
| (Visual score according to | 3 | | | 0 | 0 | 0.3 |
| German Drug Codex) | | 6 | | 0 | 0.2 | 0 |
| | | 12 | | 0.1 | — | — |
| | | 24 | | 3.1 | — | — |
| Particulate contamination | ≧10 μm: ≦6000 particles/syringe | Initial | ≧10 μm | 75 | | |
| Subvisible particles | ≧25 μm: ≦600 particles/syringe | | ≧25 μm | 7 | | |
| | | 1.5 | ≧10 μm | 68 | 158 | 147 |
| | | | ≧25 μm | 12 | 7 | 7 |
| | | 3 | ≧10 μm | 88 | 107 | 117 |
| | | | ≧25 μm | 8 | 6 | 15 |
| | | 6 | ≧10 μm | 169 | 168 | 454 |
| | | | ≧25 μm | 39 | 36 | 56 |
| | | 12 | ≧10 μm | 112 | — | — |
| | | | ≧25 μm | 16 | | |
| | | 24 | ≧10 μm | 39 | — | — |
| | | | ≧25 μm | 9 | | |
| Size Exclusion HPLC | Monomer: ≧90% | Initial | A¹ | 0.9 | | |
| | Aggregate: ≦5% | | M | 99.0 | | |
| | | | F | 0.1 | | |
| | | 1.5 | A | 1.0 | 1.3 | 2.3 |
| | | | M | 98.9 | 98.5 | 96.8 |
| | | | F | 0.1 | 0.2 | 0.9 |
| | | 3 | A | 1.0 | 1.5 | 3.1 |
| | | | M | 98.8 | 98.1 | 95.1 |
| | | | F | 0.2 | 0.4 | 1.8 |
| | | 6 | A | 1.1 | 1.8 | 4.4 |
| | | | M | 98.7 | 97.6 | 92.3 |
| | | | F | 0.2 | 0.6 | 3.3 |
| | | 12 | A | 1.1 | — | — |
| | | | M | 98.6 | | |
| | | | F | 0.3 | | |
| | | 24 | A | 1.4 | — | — |
| | | | M | 98.4 | | |
| | | | F | 0.2 | | |

| Cation Exchange HPLC | The predominant | Initial | Conforms to reference | | |
|---|---|---|---|---|---|
| | chromatographic pattern | | 88.5 | | |
| | conforms to that of the | | 0.5 | | |
| | reference material. | | 11.1 | | |
| | Main Isoforms ≧80% | 1.5 | Conforms | Conforms | Conforms not |
| | Basic Species ≦10% | | 88.0 | 86.6 | 79.6 |
| | Acidic Species ≦20% | | 0.6 | 0.6 | 0.7 |
| | | | 11.4 | 12.8 | 19.8 |
| | | 3 | Conforms | Conforms | Conforms not |
| | | | 87.7 | 85.2 | 73.3 |
| | | | 0.7 | 0.8 | 0.9 |
| | | | 11.6 | 14.0 | 25.8 |
| | | 6 | Conforms | Conforms | Conforms not |
| | | | 88.0 | 83.5 | 64.1 |
| | | | 0.6 | 0.7 | 1.0 |
| | | | 11.4 | 15.8 | 34.9 |
| | | 12 | Conforms | — | — |
| | | | 87.5 | | |
| | | | 0.5 | | |
| | | | 12.0 | | |
| | | 24 | Conforms | — | — |
| | | | 89.4 | | |
| | | | 0.1 | | |
| | | | 10.4 | | |
| Biological Activity ELISA | 70-143% | Initial | 94 | | |
| | | 1.5 | 108 | 101 | 90 |
| | | 3 | 95 | 94 | 71 |
| | | 6 | 87 | 83 | 63 |
| Protein concentration | 90-110 mg/mL | Initial | 95 | | |
| (OD 280 nm) | | 1.5 | 96 | 96 | 93 |
| | | 3 | 97 | 96 | 98 |
| | | 6 | 98 | 98 | 99 |
| | | 12 | 99 | | |
| | | 24 | 98 | | |

¹A: aggregates; M: monomer; F: fragments

### Example 7

### Methods and Materials for Cleavage Studies

### Example 7.1

### Materials

Methionine, histidine, arginine, mannitol, polysorbate 80, poloxamer 188, sodium chloride, phosphate, acetate, desferrioxamine, EDTA, sodium citrate, tris-hydrochloride, desferrithiocin, superoxide dismutase, and butyl hydroxytoluene of the highest grade were purchased from Sigma-Aldrich (St. Louis, Mo., USA). N-glycanase was purchased from Prozyme (San Leandro, Calif.). Iron (II) sulfate-7H₂O, magnesium sulfate, nickel (II) sulfate, cobalt (II) sulfate, and manganese (II) sulfate were purchased from Sigma-Aldrich (St. Louis, Mo., USA). Ferric chloride-6H₂O was purchased from Mallinckrodt (Phillipsburg, N.J., USA). Cupric sulfate-5H₂O was purchased from EMD Chemicals (Gibbstown, N.J., USA). Zinc sulfate-7H₂O was purchased from JT Baker (Phillipsburg, N.J., USA). The C18 trap was purchased from Michrom BioResources (Auburn, Calif., USA) and the capillary: bare uncoated capillary (50 μm id, 30 cm total length) and SDS MW sample buffer were purchased from Beckman Coulter (Fullerton, Calif., USA).

### Example 7.2

### Methods

### Example 7.2.1

### Deglycosylation of Antibody

Samples were enzymatically deglycosylated using N-glycanase to simplify the mass spectrum. About 30 μl of each sample (concentration about 1 g/mL) was added to 2 μl of 10% w/w n-octylglucoside and 2 μl of N-glycanase and the samples were incubated at 37° C. for 19 hours.

### Example 7.2.2

### Size Exclusion Chromatography

SEC was performed by using either of the two methods described below. (a) A Pharmacia Superdex 200 (10/300 GL) column (GE Healthcare, Piscataway, N.J.) was used for separating antibody fragment and aggregates from monomers. Separation was carried out under isocratic conditions using 211 mM Na₂SO₄ with 92 mM Na₂HPO₄, pH 7.0. Detection was performed at 214 nm and the flow rate maintained at 0.5 mL/minute. Typically, about 100 μl of a 1 mg/ml solution (100 μg load) was injected onto a column. Material fractionated from the column was concentrated and exchanged into 50 mM ammonium bicarbonate using a 10 kD Amicon Ultra-15 Centrifugal Filter Device (Millipore, USA). Fractionated material was typically re-injected onto the SEC column using the same method but with a smaller injection volume (20 μl of 1 mg/ml, 20 μg load). (b) A TSK Gel G3000 SWXL (Tosoh Bioscience) was used alternatively to monitor aggregates and fragments of the antibody. Separation was carried out under isocratic conditions using 211 mM Na₂SO₄ with 92 mM Na₂HPO₄, pH 7.0. Detection was performed at 214 nm and the flow rate maintained at 0.25 mL/minute. Typically, about 10 μl of a 2 mg/ml solution (20 μg load) was injected onto a column.

### Example 7.2.3

### Mass Spectrometry

Samples were analyzed on an API QSTAR pulsar QTOF mass spectrometer (Applied Biosystems, Foster City, Calif., USA) coupled to an Agilent 1100 capillary HPLC system (Agilent Technologies, Santa Clara, Calif., USA). The samples were introduced into the mass spectrometer and desalted using a C18 micro trap from Michrom BioResources (Auburn, Calif., USA). The samples were loaded under aqueous conditions (0.02% TFA, 0.08% formic acid in water) for the first five minutes to remove salts and then eluted under organic conditions (0.02% TFA, 0.08% formic acid in acetonitrile). Samples were run at an approximate concentration of 1 mg/mL, 10 μl injection for a 10 μg load. To help simplify the mass spectrum the samples were treated with 50 mM dithiothreitol (DTT) at room temperature for 30 minutes to reduce the disulfide bonds and release the light chain and heavy chain components. Alternatively, the samples were run non-reduced and deglycosylated to simplify the mass spectrum. To 30 μl (approximate concentration=1 mg/mL) of each sample was added 2 μl of 10% w/w n-octylglucoside and 2 μl of N-glycanase (Prozyme) and incubated at 37° C. for 19 hours. The mass spectrometer was set to run in a positive ion mode with a capillary voltage of 4500, m/z scan range of 1500-3500 for non-reduced and 500 to 2500 for the reduced samples. The instrument was tuned and calibrated using renin substrate peptide (Sigma Catalog No. R-8129). The deconvolution of the ESI mass spectra was performed using BioAnalyst software version 1.1.

### Example 7.2.4

### Capillary Electrophoresis

All studies were carried out on a Proteomelab PA800 CE system or a P/ACE MDQ system (Beckman Coulter, Inc, Fullerton, Calif.) and detection was performed at 214 nm. A bare uncoated capillary was used for the separation with dimensions of 50 μm id×30 cm total length (Beckman Coulter part number 338451) with a 0.2 micron detector window. Sample preparation was carried out under non-reducing conditions. About 100 μg of the sample was added to a 0.5 mL vial and the appropriate volume of Milli-Q water was added to obtain a final volume of 100 μl. 5 μl of 500 mM iodoacetamide was then added, followed by 50 μl of 50 mM Acetate pH 4, 1% SDS buffer for a final concentration of 1 mg/mL. The sample was mixed well and incubated at 60° C. for 10 minutes. The sample was finally transferred to an autosampler vial and placed in the 10° C. autosampler awaiting analysis. The method parameters for pre-run conditioning of the capillary were (using reverse flow) a basic rinse (0.1N sodium hydroxide) for 3 minutes at 70 psi followed by an acid rinse (0.1N hydrochloric acid) for 3 minutes at 70 psi, followed by a water rinse (Milli-Q water) for 1 minute at 70 psi, followed by a SDS-Gel fill (SDS MW Gel Buffer, Beckman Catalog No. 391163) for 10 minutes at 70 psi, followed by a Milli-Q water dip to clean the capillary. The sample was electrokinetically injected for 10 seconds at 15 kV followed by a Milli-Q water dip to clean the capillary. The voltage separation was for 35 minutes at 15 kV. The capillary temperature was 20-25° C. and the sample storage temperature was at 10° C.

### Example 7.2.5

### ICP-MS

Samples were submitted for low-resolution ICP-MS to QTI-Intertek (Whitehouse, N.J., USA) and high-resolution ICP-MS to AQura GmbH (Rodenbacher Chaussee 4, D-63457 Hanau, Germany). For low resolution ICP-MS, a Perkin Elmer Elan ICP-MS spectrometer was used whereas for high-resolution the HR-ICP-MS Thermo Element XR was used.

### Example 7.2.6

### Filtration

### Example 7.2.6.1

Ultrafiltration (UF) is a type of membrane filtration where hydrostatic pressure forces a liquid against a semipermeable membrane. The antibody is retained, while water and low molecular weight solutes such as the iron salts pass through the membrane. A Millipore 30 K Pellicon 2 regenerated cellulose membrane was installed as per Millipore's instructions. The manufacturer's torque specifications were maintained and the UF system was set up with the appropriate pressure gauges; tubing, and pumps. The appropriate valving was then opened to begin ultrafiltration. The inlet (feed) pressure and retentate pressure were maintained within the ranges specified and the permeate flow rate and pressures were closely monitored. Data was recorded every 15-30 minutes. After ultrafiltration was complete the final weight was recorded and the concentration determined by A₂₈₀.

### Example 7.2.6.2

Diafiltration (DF) is a tangential flow filtration process that is performed in conjunction with a filtration operation (usually UF), where buffer is added to replace the amount of solution lost through the filter to maintain a constant volume. DF is used to remove metals and replace the original solution with a new buffer. Fluid is pumped tangentially along the surface of the membrane (Millipore 30 K Pellicon 2 Regenerated Cellulose Membranes per Millipore instructions). Steady pressure is applied to force a portion of the fluid through the membrane to the filtrate side. As in UF, the IgG molecules are too large to pass through the membrane pores and are retained on the upstream side. The retained components do not build up at the surface of the membrane. Instead, they are swept along by the tangential flow. At least 8 diavolumes are used to remove iron.

### Example 8

### Fragmentation Analysis

### Example 8.1

### Fragmentation of an IgG Molecule (J695) in the Hinge Region

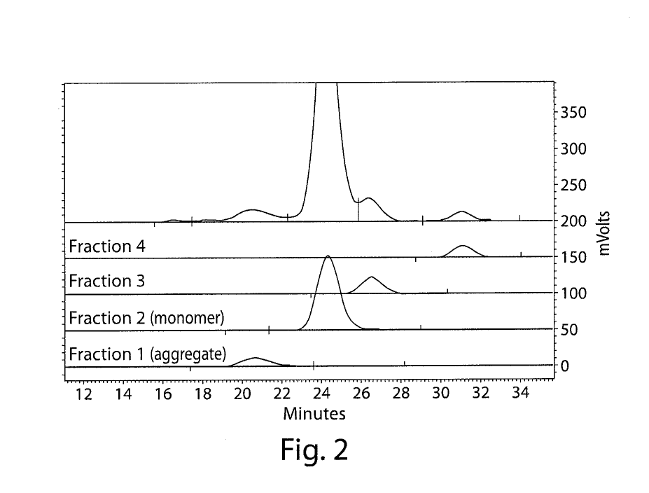

SEC is commonly used to monitor the decrease in the monomer peak and the appearance of additional peaks in a chromatogram. FIG. 2 shows a typical SEC profile of a monoclonal antibody after storage at 40° C. for about 6 months. Four fractions (fractions 1-4) were collected and subsequently analyzed by SDS-PAGE, MS and CE-SDS. Fractions 1 and 2 represent aggregate and monomer antibody, respectively. Fraction 3 contains a 100 kDa species formed by the loss of an Fab arm (Fab+Fc or fragment 2) and a low percentage of a non-reducible (NR) species composed of a thioether linkage between heavy (HC) and light chains (LC) (Tous, G. I. et al. (2005) Anal. Chem. 77(9):2675-82). Fraction 4 contains the Fab arm (Cordoba, A. J. et al. (2005) J. Chromatogr. B Analyt. Technol. Biomed. Life Sci. 818(2):115-21).

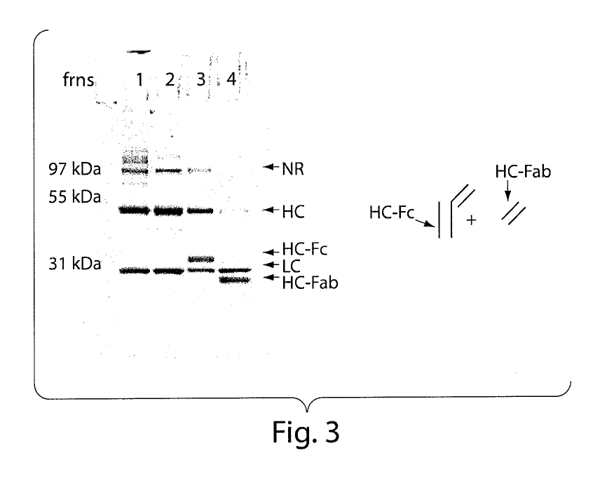

Analysis of aggregate and monomer by SDS-PAGE, under reducing conditions (FIG. 3, lanes 1 and 2), showed HC, LC and an NR species with an apparent mass of 100 kDa. Higher order aggregates that are non-reducible are found in fraction 1 and to a small extent in fraction 2. Analysis of fraction 3 under reducing conditions (lane 3) showed HC, LC, NR species and the Fc fragment of the heavy chain (HC-Fc). Analysis of fraction 4 (lane 4) showed the LC and the Fab fragment of the HC(HC-Fab).

Fractions 3 and 4 were also analyzed by ESI/LC-MS. FIG. 4 shows spectra obtained after deglycosylation of fraction 3. Multiple cleavage sites are observed in the hinge region of the heavy chain of the IgG molecule that resulted in the loss of the Fab arm (Peaks a-e, summarized in Table 9). The major sites of cleavage are observed in Peak-a between residues His-222 and Thr-223 (H/T) and Peak-e between residues Cys-218 and Asp-219 (C/D). Minor cleavage sites are found between T/H, K/T, and D/K. The cleavage site between Ser-217 and Cys-218 (S/C) was not found nor was the addition of 70 Da to the aspartic acid residue on the HC, previously reported at the higher pH of 8 by Cohen et al. et al. (2007) J. Am. Chem. Soc. 129(22):6976-7.

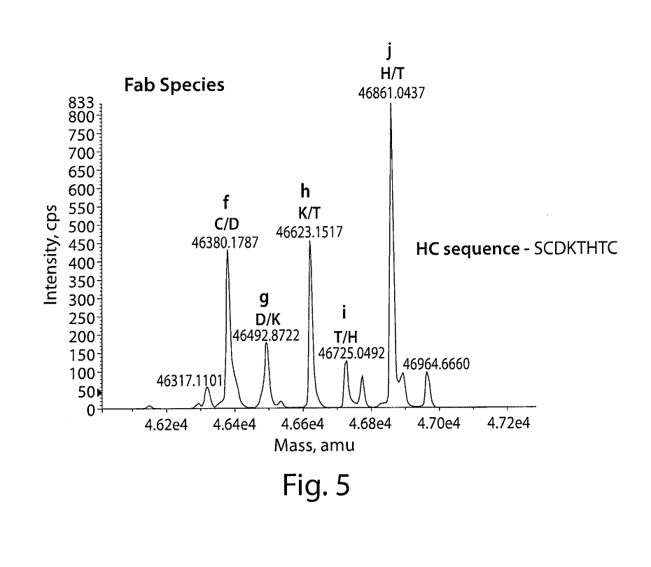

FIG. 5 shows MS spectra obtained from fraction 4, which contained the corresponding Fab species (peaks f-j, summarized in Table 10).

TABLE 10
Summary of ESI/LC-MS Spectra Of The Different Fragments After
Separation By SEC

| | | Theo. | Theo. | Observed | Cleavage |
|---|---|---|---|---|---|
| Peak | Residues | Glutamine | Pyroglutamate | mass | site |
| a | HC: 223-444 | 96804.1 | 96770.1 | 96781.0 | H/T |
| b | HC: 222-444 | 96941.2 | 96907.2 | 96914.0 | T/H |
| c | HC: 221-444 | 97042.3 | 97008.3 | 97022.1 | K/T |
| d | HC: 220-444 | 97170.5 | 97136.5 | 97157.3 | D/K |
| e | HC: 219-444 | 97285.6 | 97251.6 | 97268.0 | C/D |
| f | HC: 1-218 | — | 46377.9 | 46380.2 | C/D |
| g | HC: 1-219 | — | 46493.0 | 46492.9 | D/K |
| h | HC: 1-220 | — | 46621.1 | 46623.2 | K/T |
| i | HC: 1-221 | — | 46722.2 | 46725.0 | T/H |
| j | HC: 1-222 | — | 46859.4 | 46861.0 | H/T |
| k | LC: 1-215 | — | 22927.5 | 22927.9 | E/C |
| l | HC: 1-217 | — | 23159.0 | 23159.3 | S/C |

Major sites of cleavage are also seen in Peak (f) between C/D residues and Peak (j) between the H/T residues. Minor cleavage sites between D/K and T/H are found with a higher level of cleavage between K/T when compared to fragment 2 spectra. Also shown in FIG. 6 (peaks k and L, summarized in Table 10) is the presence of free light chain fragments (Peak (k)—residues 1-215) and heavy chain fragment (Peak (L)—residues 1-217) in Fraction 4. As noted above, neither the corresponding fragment 2 species that would contain fragment 218-444 nor the addition of 70 Da to the Asp-219 residue as reported by Cohen et al. (2007) J. Am. Chem. Soc. 129(22) 6976-7 was seen.

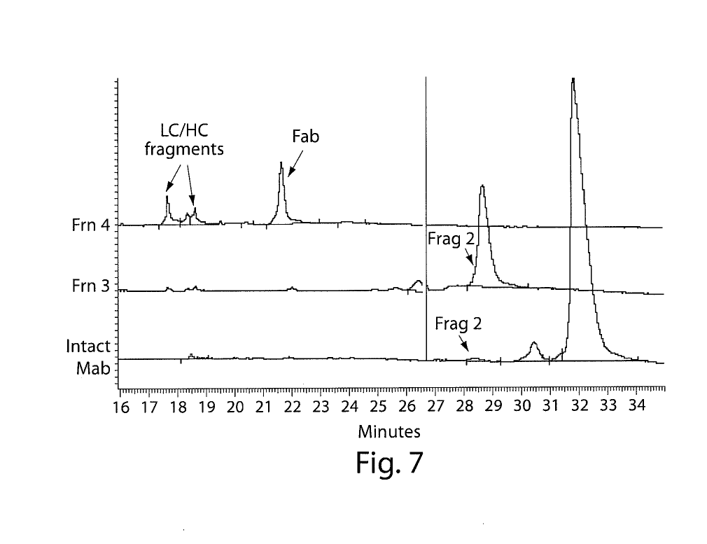

Fractions 3 and 4 were also analyzed by CE-SDS. FIG. 7 shows an electropherograms of fraction 3 and migrating position of the fragment 2 species (loss of Fab arm). As observed in the electropherogram of intact antibody, fragment 2 was well resolved from the main monomer peak as well as from other peaks, which consequently provided an accurate assessment of levels of this fragment for subsequent analysis. Fraction 4 showed intact Fab as well as LC and HC fragments.

### Example 8.2

### Presence of Iron or Copper Caused Cleavage of the IgG Molecule in a Dose Dependent Manner in the Presence of Histidine

In an embodiment, incubation of a lambda light chain containing anti-IL-12 antibody J695 lot 1 at 40° C. accelerates the fragmentation of the antibody in the hinge region when iron and histidine are present in the formulation (Table 11).

TABLE 11
Analysis By SEC Showed Enhanced Fragmentation And
Aggregation In J695 Lot 1 At 40° C.

| | Time | Percent | Percent | Percent |
|---|---|---|---|---|
| | Point | Monomer | Aggregate | Fragment |
| Mab-lot 1 | Initial | 99.4 | 0.43 | 0.17 |
| Normal lots | Initial | 99.5 ± 0.05 | 0.4 ± 0.07 | 0.1 ± 0.01 |
| 5° C. | | | | |
| Mab-lot 1 | 1 M | 99.2 | 0.58 | 0.20 |
| Normal lots | 1 M | 99.5 ± 0.05 | 0.5 ± 0.06 | 0.1 ± 0.01 |
| 25° C. | | | | |
| Mab-lot 1 | 1 M | 98.6 | 1.09 | 0.31 |
| Normal lots | 1 M | 99.0 ± 0.08 | 0.8 ± 0.08 | 0.2 ± 0.01 |
| 40° C. | | | | |
| Mab-lot 1 | 1 M | 93.1 | 2.22 | 4.73 |
| Normal lots | 1 M | 98.1 ± 0.1 | 1.4 ± 0.08 | 0.5 ± 0.04 |

At 40° C., the level of fragmentation in J695, lot 1 was as high as 4.73% when compared to an average of 0.5% obtained from 5 normal lots. Using CE-SDS the level of fragment 2 (Fab+Fc) was accurately estimated and shown to be 3 fold higher in J695 lot 1 (Table 12).

TABLE 12
Analysis By CE-SDS Of The Different Degradation Species

| | LC/HC | | | | | |
|---|---|---|---|---|---|---|
| | fragments | | HC/FAB | | Fragment 2 | |
| | Normal | | Normal | | Normal | |
| | lot | Mab-lot 1 | lot | Mab-lot 1 | lot | Mab-lot 1 |
| 40° C. | 0.45 | 1.11 | 0.58 | 0.86 | 1.59 | 4.20 |
| 25° C. | 0.16 | 0.33 | 0.30 | 0.33 | 0.84 | 1.05 |
| 5° C. | 0.17 | 0.28 | 0.28 | 0.24 | 0.74 | 0.69 |

Other degradation species were quantified by CE-SDS and the level of the Fab fragment was elevated. The levels of fragments (LC/HC fragments) were significantly elevated as well.

A number of studies conducted did not support protease activity as a cause for increased fragmentation in J695 lot 1. Incubation, for example, with a cocktail of protease inhibitors did not lower levels of fragmentation and two-dimensional gel electrophoresis and identification of host cell proteins, after removing the monoclonal antibody, also showed no evidence of contaminating proteases (results not shown).

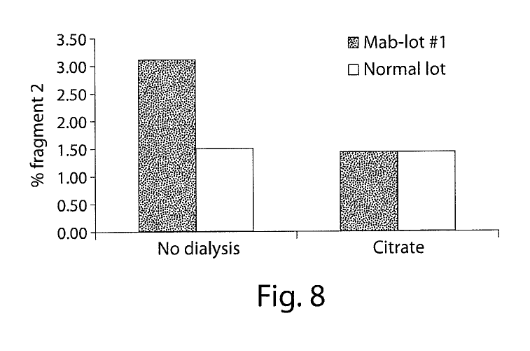

Normal levels of fragmentation were restored after dialysis against citric acid, using a 10,000 MWCO membrane at 40° C. (FIG. 8), suggesting that metals were involved in the enhanced fragmentation of J695 lot 1. A number of experiments were subsequently performed to evaluate the role of metals in fragmentation. J695 lot 1 as well as other lots were analysed for the presence of 64 different elements by ICP-MS. These studies demonstrated that J695 lot 1 had ten times the level of iron (500 ppb) when compared to 5 normal lots using high-resolution ICP-MS (Table 13).

TABLE 13
Analysis Of Iron Levels By High Resolution ICP-MS
Iron (ppb)

| | |
|---|---|
| Mab-lot #1 | 500 |
| Other lots | 63 ± 10 |

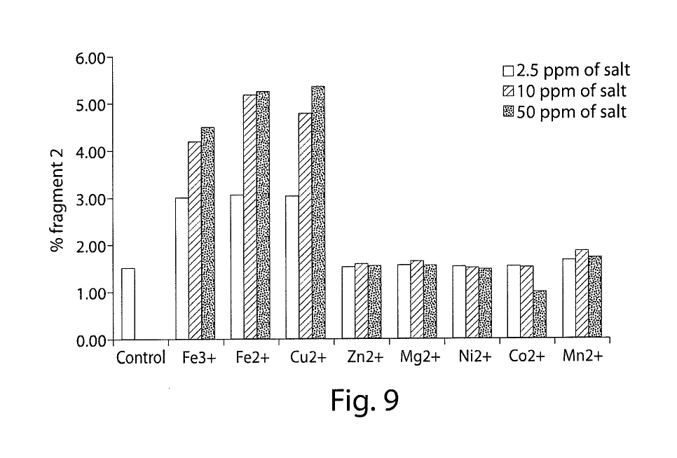

Antibody samples were spiked with different levels of metal salts (2.5, 10 and 50 ppm) into a normal lot and incubated at 40° C. As shown in FIG. 9, formulations with either oxidized states of iron or copper showed a dose dependent increase in fragmentation (fragment 2). Other metals tested had no effect on fragmentation. The level of fragmentation observed with 500 ppb of spiked iron (2.5 ppm of iron salt) was similar to that observed for J695 lot 1. Table 14 summarizes the degradation profile of the antibody induced by different metals as analysed by CE-SDS. The antibody samples were stored at 40° C. for 1 month before analysis. The level of Fab, free LC/HC fragments and fragment 2 (Fab+Fc) were all elevated in the presence of iron or copper and were unchanged in the presence of other metals.

TABLE 14
Analysis By CE-SDS Of The Fragmentation Profile
With Different Metals

| | LC/HC | | |
|---|---|---|---|
| | fragment | HC/FAB | Fragment 2 |
| 2.5 ppm | | | |
| Fe3+ | 1.40 | 0.72 | 3.02 |
| Fe2+ | 1.41 | 0.72 | 3.07 |
| Cu2+ | 1.26 | 0.72 | 3.04 |
| Zn2+ | 0.62 | 0.60 | 1.54 |
| Mg2+ | 0.65 | 0.60 | 1.58 |
| Ni2+ | 0.68 | 0.62 | 1.54 |
| Co2+ | 0.65 | 0.59 | 1.54 |
| Mn2+ | 0.65 | 0.62 | 1.66 |
| 10 ppm | | | |
| Fe3+ | 1.96 | 0.82 | 4.20 |
| Fe2+ | 2.45 | 0.92 | 5.20 |
| Cu2+ | 2.08 | 1.00 | 4.78 |
| Zn2+ | 0.66 | 0.61 | 1.59 |
| Mg2+ | 0.69 | 0.61 | 1.66 |
| Ni2+ | 0.68 | 0.59 | 1.52 |
| Co2+ | 0.68 | 0.60 | 1.52 |
| Mn2+ | 0.78 | 0.62 | 1.86 |
| 50 ppm | | | |
| Fe3+ | 2.22 | 0.92 | 4.50 |
| Fe2+ | 2.67 | 1.06 | 5.28 |
| Cu2+ | 2.56 | 1.21 | 5.37 |
| Zn2+ | 0.59 | 0.59 | 1.56 |
| Mg2+ | 0.66 | 0.60 | 1.58 |
| Ni2+ | 0.58 | 0.62 | 1.50 |
| Co2+ | 0.53 | 0.34 | 1.00 |
| Mn2+ | 0.76 | 0.63 | 1.72 |

### Example 8.3

### Chelation of Iron with Desferrioxamine, an Iron Specific Chelator, Blocked Fragmentation

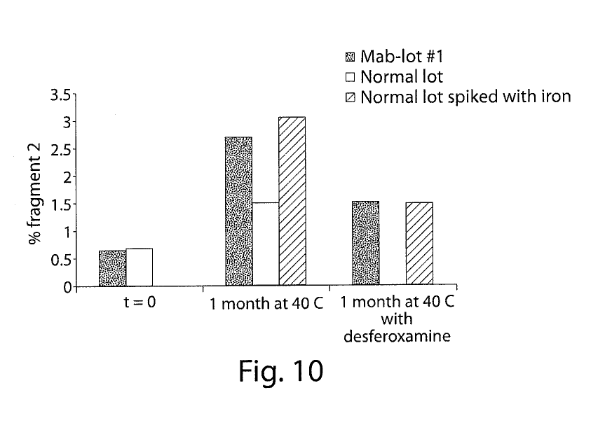

J695 lot 1 was incubated with 1 mM of desferrioxamine, an iron specific chelator. Normal levels of fragmentation were observed after incubation at 40° C. for 1 month (FIG. 10). Spiking a normal antibody lot with iron (500 ppb) showed elevated fragment levels that were restored to normal levels by pre-incubation with desferrioxamine (FIG. 10).

### Example 8.4

### Enhanced Fragmentation Catalyzed by Both Histidine and Iron

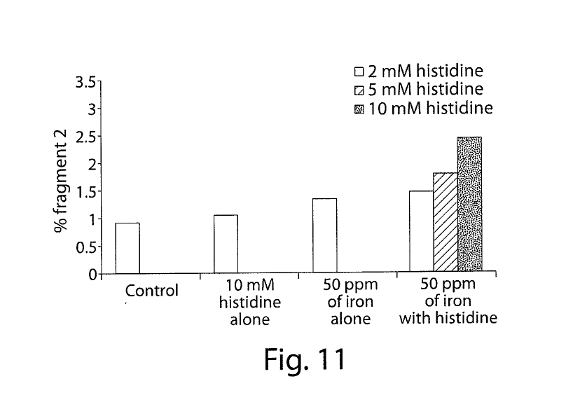

The contribution from histidine to metal induced fragmentation was investigated (FIG. 11). A normal lot of the monoclonal antibody was dialyzed against water. Iron alone (50 ppm) or histidine alone (10 mM) were added or iron (50 ppm) with different concentrations of histidine (2, 5 and 10 mM) at a constant pH of 6.0 were added to the monoclonal antibody and incubated at 40° C. for one week. As seen in FIG. 11 neither the presence of histidine nor iron alone resulted in a significant increase in antibody fragmentation over control levels. However, when antibody was incubated with iron and histidine together, a dose dependent increase in fragmentation was observed, which indicated that the level of histidine added to the formulation could play a significant role in iron induced fragmentation.

### Example 8.5

### Comparison of MS Spectra Between a Normal Stressed Lot and Metal Catalyzed Fragmentation in J695 Lot 1 Show a Distinct Cleavage Profile

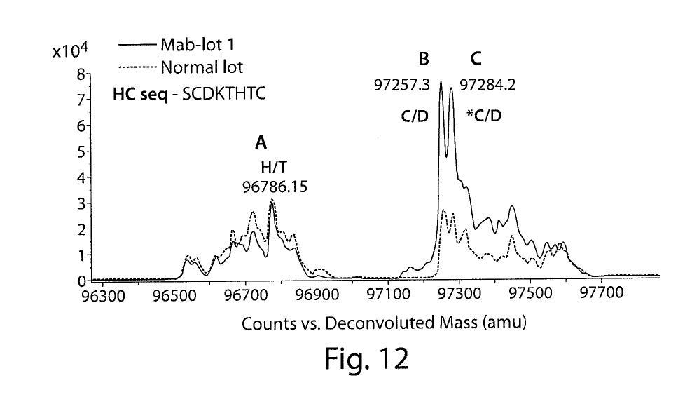

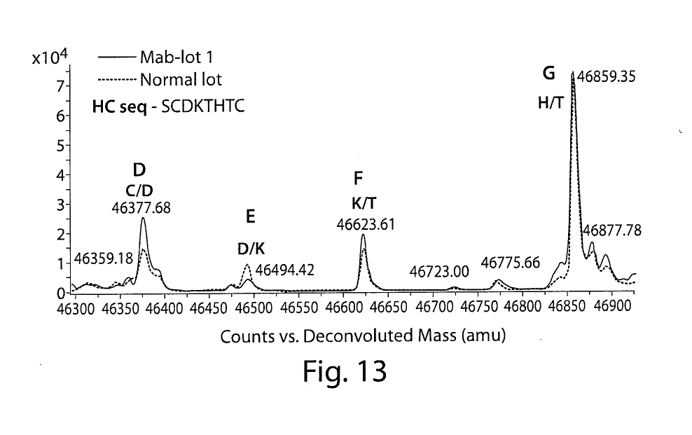

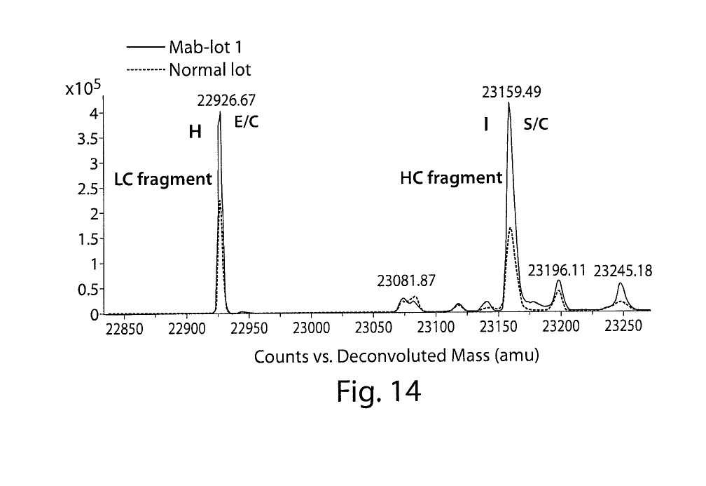

FIG. 12 shows a comparison of MS spectra after deglycosylation of fragment 2 (Fab+Fc). Cleavage between Cys-218 and Asp-219 (C/D) in the hinge region sequence SCDKTHTC was significantly elevated in J695 lot 1 whereas cleavage at other cleavage sites on the molecule was not increased. However, analysis of the Fab species (FIG. 13) showed that levels of the corresponding Fab fragment at this cleavage site (residues 1-218) in J695 lot 1 was comparable to that of a normal stressed lot, whereas free HC fragment cleaved between Ser-217 and Cys-218 (S/C) was significantly elevated giving an HC fragment from residues 1-217 (FIG. 14). Cohen et al. ((2007) J. Am. Chem. Soc. 129(22) 6976-7) have recently demonstrated that cleavage between the S/C bond occurs via a β-elimination mechanism. This mechanism is prevalent at higher pH (pH 8) and is preceded by the breaking of the LC-HC disulfide bond and subsequent hydrolysis of the dehydroalanine residue resulting in an Fab fragment that ends with serine amide (addition of 1 Da mass) and a C-terminal Fc fragment with a pyruvoyl group (addition of 70 Da mass to the aspartic residue). Results indicated an increase in the cleavage site between residues C/D and an addition of 27 Da to the aspartic acid residue (Peak C in Table 14) suggesting a different mechanism of hydrolysis. Elevated levels of free light chain that is cleaved between E/C (residues 1-215) in J695 lot 1 (FIG. 14) were observed. Table 15 summarizes the data collected for comparison of the different MS spectra.

TABLE 15
Summary Of ESI/LC-MS Spectra Of The Different Fragments

| | | Theo. | Theo. | Observed | Cleavage |
|---|---|---|---|---|---|
| Peak | Residues | Glutamine | Pyroglutamate | mass | site |
| A | HC: 223-444 | 96804.1 | 96770.1 | 96777.0 | H/T |
| B | HC: 219-444 | 97285.6 | 97251.6 | 97257.3 | C/D |
| C | HC: 219-444 | | | 97284.2 | C/D |
| D | HC: 1-218 | 46377.9 | | 46377.7 | C/D |
| E | HC: 1-219 | 46493.0 | | 46494.4 | D/K |
| F | HC: 1-220 | 46621.1 | | 46623.6 | K/T |
| G | HC: 1-222 | 46859.4 | | 46859.4 | H/T |
| H | LC: 1-215 | 22927.5 | | 22926.7 | E/C |
| I | HC: 1-217 | 23159.0 | | 23159.5 | S/C |

### Example 8.6

### Cleavage Mechanism is Specific for Molecules that Contain a Lambda Chain

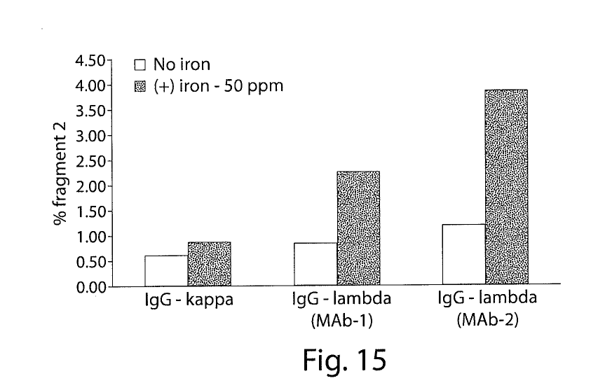

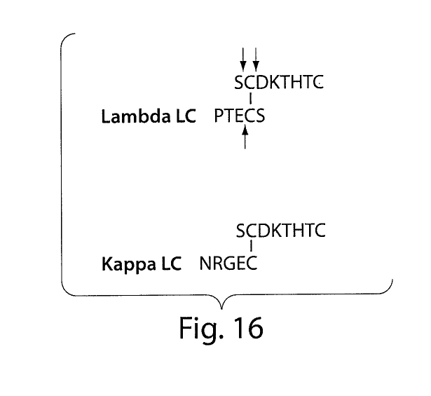

The ability of iron and histidine to catalyze hydrolysis of antibody molecules that possessed either kappa or lambda light chain was investigated. Two IgG molecules with lambda LCs were cleaved by iron and histidine whereas IgG molecules with a kappa LCs were not cleaved (FIG. 15). FIG. 16 shows the sequence of residues around which hydrolysis of the IgG molecule is observed.

### Example 9

### Accelerated Stability Studies

In an embodiment, incubation of a lambda light chain containing anti-IL-12 antibody J695 at 40° C. accelerates the fragmentation of the antibody in the hinge region when iron and histidine are present in the formulation. Consequently, an incubation temperature of 40° C. for these studies was chosen. In order to clearly differentiate between fragmentation of the antibody induced by temperature per se and fragmentation induced by the presence of iron and histidine, all accelerated stability studies were designed and performed such that a positive control (i.e., the antibody formulation containing iron and histidine) was blanked by a reference formulation (i.e., the respective formulation containing histidine, but lacking iron).

All the various J695 formulations tested in the experiments listed in Examples 9.1 through 9.15 were filled in sterile, non-pyrogenic, polypropylene cryogenic vials and incubated at 40° C. for up to 3 months. At predetermined points of time (i.e., at TO, after T1 month and after T3 months of storage at 40 C/75% RH), samples of all formulations were pulled, and the extent of antibody fragmentation in the various formulations was determined by SEC as described in 7.2.2.

### Example 9.1

### Fragmentation of J695 in the Presence of Iron at Solution pH 5

Antibody J695 was formulated at 2 mg/mL, pH 5.0 in the following compositions:
a) 10 mM methionine, 10 mM histidine, 40 mg/mL mannitol, 0.01% (m/v) polysorbate 80; and
b) 10 mM methionine, 10 mM histidine, 40 mg/mL mannitol, 0.01% (m/v) polysorbate 80 and 0.5 ppm iron.

Additionally, antibody J695 was formulated at 100 mg/mL, pH 5.0 in the following compositions:
c) 10 mM methionine, 10 mM histidine, 40 mg/mL mannitol, 0.01% (m/v) polysorbate 80; and
d) 10 mM methionine, 10 mM histidine, 40 mg/mL mannitol, 0.01% (m/v) polysorbate 80, 0.5 ppm iron.

The results of these studies are listed in Table 15.1. The results demonstrate that the presence of iron and histidine in J695 formulations promotes J695 fragmentation as compared to the control (i.e., the formulation lacking iron) at 2 mg/mL. However, the results also demonstrate that reduction to pH 5 protected J695 from iron-histidine mediated fragmentation. This reduction in fragmentation was not observed at pH 6.0 or pH 7.0 (see, e.g., Tables 15.2 and Table 15.3 below).

TABLE 15.1
Fragment levels of J695 with different formulations
during accelerated stability studies.

| | | T1 month, | T3 months, |
|---|---|---|---|
| Formulation composition | T0, 5° C. | 40° C. | 40° C. |
| pH 5, 2 mg/mL J695, 10 mM methionine, 10 mM | 3.9 | 4.5 | 11.4 |
| histidine, 40 mg/mL mannitol, 0.01% (m/v) polysorbate 80 | | | |
| pH 5, 2 mg/mL J695, 10 mM methionine, 10 mM | 4.3 | 6.1 | 18.9 |
| histidine, 40 mg/mL mannitol, 0.01% (m/v) polysorbate | | | |
| 80, 0.5 ppm iron | | | |
| pH 5, 100 mg/mL J695, 10 mM methionine, 10 mM | 3.2 | 5.6 | 14.1 |
| histidine, 40 mg/mL mannitol, 0.01% (m/v) polysorbate 80 | | | |
| pH 5, 100 mg/mL 1695, 10 mM methionine, 10 mM | 3.2 | 5.8 | 14.8 |
| histidine, 40 mg/mL mannitol, 0.01% (m/v) polysorbate | | | |
| 80, 0.5 ppm iron | | | |

### Example 9.2

### Fragmentation of J695 in the Presence of Iron at Solution pH 6

Antibody J695 was formulated at 2 mg/mL, pH 6.0 in the following compositions:
a) 10 mM methionine, 10 mM histidine, 40 mg/mL mannitol, 0.01% (m/v) polysorbate 80;
b) 10 mM methionine, 10 mM histidine, 40 mg/mL mannitol, 0.01% (m/v) polysorbate 80 and 0.5 ppm iron; and
c) 10 mM methionine, 10 mM histidine, 40 mg/mL mannitol, 0.01% (m/v) polysorbate 80 and 2.5 ppm iron.

Additionally, antibody J695 was formulated at 100 mg/mL, pH 6.0 in the following compositions:
d) 10 mM methionine, 10 mM histidine, 40 mg/mL mannitol, 0.01% (m/v) polysorbate 80;
e) 10 mM methionine, 10 mM histidine, 40 mg/mL mannitol, 0.01% (m/v) polysorbate 80, 0.5 ppm iron; and
f) 10 mM methionine, 10 mM histidine, 40 mg/mL mannitol, 0.01% (m/v) polysorbate 80, 2.5 ppm iron.

The results of these studies are listed in Table 15.2. The results demonstrate that the presence of iron and histidine in J695 formulations leads to substantial J695 fragmentation as compared to the control (i.e., the formulation lacking iron) at both 2 mg/mL and 100 mg/mL J695. The results further demonstrate that an increase in iron levels results in increased J695 fragment levels.

TABLE 15.2
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 10 mM methionine, 10 mM | 3.3 | 4.2 | 11.0 |
| histidine, 40 mg/mL mannitol, 0.01% (m/v) polysorbate 80 | | | |
| pH 6, 2 mg/mL J695, 10 mM methionine, 10 mM | 3.5 | 14.0 | 27.9 |
| histidine, 40 mg/mL mannitol, 0.01% (m/v) polysorbate | | | |
| 80, 0.5 ppm iron | | | |
| pH 6, 2 mg/mL J695, 10 mM methionine, 10 mM | 3.5 | 15.0 | 28.6 |
| histidine, 40 mg/mL mannitol, 0.01% (m/v) polysorbate | | | |
| 80, 2.5 ppm iron | | | |
| pH 6, 100 mg/mL J695, 10 mM methionine, 10 mM | 3.1 | 3.9 | 10.8 |
| histidine, 40 mg/mL mannitol, 0.01% (m/v) polysorbate 80 | | | |
| pH 6, 100 mg/mL J695, 10 mM methionine, 10 mM | 3.6 | 7.2 | 18.4 |
| histidine, 40 mg/mL mannitol, 0.01% (m/v) polysorbate | | | |
| 80, 0.5 ppm iron | | | |
| pH 6, 100 mg/mL J695, 10 mM methionine, 10 mM | 3.1 | 9.3 | 23.7 |
| histidine, 40 mg/mL mannitol, 0.01% (m/v) polysorbate | | | |
| 80, 2.5 ppm iron | | | |

### Example 9.3

### Fragmentation of J695 in the Presence of iron at Solution pH 7

Antibody J695 was formulated at 2 mg/mL, pH 7.0 in the following compositions:
a) 10 mM methionine, 10 mM histidine, 40 mg/mL mannitol, 0.01% (m/v) polysorbate 80; and
b) 10 mM methionine, 10 mM histidine, 40 mg/mL mannitol, 0.01% (m/v) polysorbate 80 and 0.5 ppm iron.
   Additionally, antibody J695 was formulated at 100 mg/mL, pH 7.0 in the following compositions:
c) 10 mM methionine, 10 mM histidine, 40 mg/mL mannitol, 0.01% (m/v) polysorbate 80; and
d) 10 mM methionine, 10 mM histidine, 40 mg/mL mannitol, 0.01% (m/v) polysorbate 80, 0.5 ppm iron.

The results of these studies are listed in Table 15.3. The results demonstrate that the presence of iron and histidine in J695 formulations fosters J695 fragmentation as compared to the control (i.e., the formulation lacking iron) over a broad protein concentration range, i.e., 2 mg/mL up to 100 mg/mL. The results further demonstrate that fragmentation of J695 as a consequence of iron-histidine mediated fragmentation is dependent on the pH of the formulation. As shown in Table 15.3, formulations having a pH above 6.0 (Table 15.3) are more prone to fragmentation. At 100 mg/mL and at pH 7.0 fragmentation plateaus, whereas at 2 mg/mL it continues to increase at pH 7.0.

TABLE 15.3
Fragment levels of J695 with different formulations during accelerated
stability studies.

| | | T1 month, | T3 months, |
|---|---|---|---|
| Formulation composition | T0, 5° C. | 40° C. | 40° C. |
| pH 7, 2 mg/mL J695, 10 mM methionine, 10 mM | 3.5 | 6.1 | 15.4 |
| histidine, 40 mg/mL mannitol, 0.01% (m/v) polysorbate 80 | | | |
| pH 7, 2 mg/mL J695, 10 mM methionine, 10 mM | 3.2 | 25.6 | 49.8 |
| histidine, 40 mg/mL mannitol, 0.01% (m/v) polysorbate | | | |
| 80, 0.5 ppm iron | | | |
| pH 7, 100 mg/mL J695, 10 mM methionine, 10 mM | 3.3 | 4.9 | 13.9 |
| histidine, 40 mg/mL mannitol, 0.01% (m/v) polysorbate 80 | | | |
| pH 7, 100 mg/mL J695, 10 mM methionine, 10 mM | 2.9 | 10.6 | 24.0 |
| histidine, 40 mg/mL mannitol, 0.01% (m/v) polysorbate | | | |
| 80, 0.5 ppm iron | | | |

### Example 9.4

### Fragmentation of J695 at Conditions of Various Ionic Strength

Antibody J695 was formulated at 2 mg/mL, pH 6.0 in the following compositions:
a) 10 mM methionine, 10 mM histidine, 40 mg/mL mannitol, 0.01% (m/v) polysorbate 80;
b) 10 mM methionine, 10 mM histidine, 40 mg/mL mannitol, 0.01% (m/v) polysorbate 80 and 0.5 ppm iron;
c) 10 mM methionine, 10 mM histidine, 40 mg/mL mannitol, 0.01% (m/v) polysorbate 80 and 150 mM of NaCl; and
d) 10 mM methionine, 10 mM histidine, 40 mg/mL mannitol, 0.01% (m/v) polysorbate 80, 0.5 ppm iron and 150 mM NaCl.
   Additionally, J695 was formulated at 100 mg/mL, pH 6.0 in the compositions (a) to (d) as listed above.

The results of these studies are listed in Table 15.4. The results demonstrate that the presence of iron and histidine in J695 formulations leads to substantial J695 fragmentation as compared to the control (i.e., the formulation lacking iron) at both 2 mg/mL and 100 mg/mL J695. The results further demonstrate that ionic strength does not impact iron-histidine mediated fragmentation of J695.

TABLE 15.4
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 10 mM methionine, 10 mM | 3.3 | 4.2 | 11.0 |
| histidine, 40 mg/mL mannitol, 0.01% (m/v) polysorbate 80 | | | |
| pH 6, 2 mg/mL J695, 10 mM methionine, 10 mM | 3.5 | 14.0 | 27.9 |
| histidine, 40 mg/mL mannitol, 0.01% (m/v) polysorbate | | | |
| 80, 0.5 ppm iron | | | |
| pH 6, 100 mg/mL J695, 10 mM methionine, 10 mM | 3.1 | 3.9 | 10.8 |
| histidine, 40 mg/mL mannitol, 0.01% (m/v) polysorbate 80 | | | |
| pH 6, 100 mg/mL J695, 10 mM methionine, 10 mM | 3.6 | 7.2 | 18.4 |
| histidine, 40 mg/mL mannitol, 0.01% (m/v) polysorbate | | | |
| 80, 0.5 ppm iron | | | |
| pH 6, 2 mg/mL J695, 10 mM methionine, 10 mM | 2.9 | 4.9 | 13.5 |
| histidine, 40 mg/mL mannitol, 0.01% (m/v) polysorbate | | | |
| 80, 150 mM NaCl | | | |
| pH 6, 2 mg/mL J695, 10 mM methionine, 10 mM | 2.6 | 9.6 | 20.2 |
| histidine, 40 mg/mL mannitol, 0.01% (m/v) polysorbate | | | |
| 80, 0.5 ppm iron, 150 mM NaCl | | | |
| pH 6, 100 mg/mL J695, 10 mM methionine, 10 mM | 2.7 | 4.2 | 11.9 |
| histidine, 40 mg/mL mannitol, 0.01% (m/v) polysorbate | | | |
| 80, 150 mM NaCl | | | |
| pH 6, 100 mg/mL J695, 10 mM methionine, 10 mM | 2.6 | 6.7 | 18.0 |
| histidine, 40 mg/mL mannitol, 0.01% (m/v) polysorbate | | | |
| 80, 0.5 ppm iron, 150 mM NaCl | | | |

### Example 9.5

### Fragmentation of J695 Formulated in Arginine Buffer in the Presence of Iron and Histidine at Solution pH 6

Antibody J695 was formulated at 2 mg/mL, pH 6.0 in the following compositions:
a) 30 mM arginine, 10 mM histidine, 40 mg/mL mannitol, 0.01% (m/v) polysorbate 80; and
b) 30 mM arginine, 10 mM histidine, 40 mg/mL mannitol, 0.01% (m/v) polysorbate 80 and 0.5 ppm iron.
   Additionally, J695 was formulated at 100 mg/mL, pH 6.0 in the following compositions:
c) 30 mM arginine, 10 mM histidine, 40 mg/mL mannitol, 0.01% (m/v) polysorbate 80; and
d) 30 mM arginine, 10 mM histidine, 40 mg/mL mannitol, 0.01% (m/v) polysorbate 80, 0.5 ppm iron.

The results of these studies are listed in Table 15.5. The results demonstrate that the presence of iron and histidine in J695 formulations results in J695 fragmentation as compared to the control (i.e., the formulation lacking iron) and that the presence of other organic and amino acid based buffers, such as arginine, does not impact iron-histidine mediated fragmentation of J695, regardless of protein concentration (e.g., 2 mg/mL or 100 mg/mL).

TABLE 15.5
Fragment levels of J695 formulations during accelerated stability studies.

| | | T1 month, | T3 months, |
|---|---|---|---|
| Formulation composition | T0, 5° C. | 40° C. | 40° C. |
| pH 6, 2 mg/mL J695, 30 mM arginine, 10 mM histidine, | 3.2 | 7.1 | 15.0 |
| 40 mg/mL mannitol, 0.01% (m/v) polysorbate 80 | | | |
| pH 6, 2 mg/mL J695, 30 mM arginine, 10 mM histidine, | 3.3 | 10.3 | 23.2 |
| 40 mg/mL mannitol, 0.01% (m/v) polysorbate 80, 0.5 ppm | | | |
| iron | | | |
| pH 6, 100 mg/mL J695, 30 mM arginine, 10 mM histidine, | 2.2 | 4.7 | 13.8 |
| 40 mg/mL mannitol, 0.01% (m/v) polysorbate 80 | | | |
| pH 6, 100 mg/mL J695, 30 mM arginine, 10 mM histidine, | 2.5 | 7.7 | 20.5 |
| 40 mg/mL mannitol, 0.01% (m/v) polysorbate 80, 0.5 ppm | | | |
| iron | | | |

### Example 9.6

### Fragmentation of J695 Formulated in Phosphate Buffer in the Presence of Iron and Histidine at Solution pH 6

Antibody J695 was formulated at 2 mg/mL, pH 6.0 concentration in the following compositions:
a) 30 mM phosphate, 10 mM histidine, 40 mg/mL mannitol, 0.01% (m/v) polysorbate 80; and
b) 30 mM phosphate, 10 mM histidine, 40 mg/mL mannitol, 0.01% (m/v) polysorbate 80 and 0.5 ppm iron.
   Additionally, antibody J695 was formulated at 100 mg/mL, pH 6.0 in the following compositions:
c) 30 mM phosphate, 10 mM histidine, 40 mg/mL mannitol, 0.01% (m/v) polysorbate 80; and
d) 30 mM phosphate, 10 mM histidine, 40 mg/mL mannitol, 0.01% (m/v) polysorbate 80, 0.5 ppm iron.

The results of these studies are listed in Table 15.6. The results demonstrate that the presence of iron and histidine in J695 formulations does not result in J695 fragmentation both at 2 mg/mL and at 100 mg/mL. The results further demonstrate that the use of phosphate in antibody formulations reduces iron-histidine mediated fragmentation of antibodies, regardless of protein concentration (e.g., 2 mg/mL or 100 mg/mL).

TABLE 15.6
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 30 mM phosphate, 10 mM histidine, | 2.5 | 9.3 | 20.5 |
| 40 mg/mL mannitol, 0.01% (m/v) polysorbate 80 | | | |
| pH 6, 2 mg/mL J695, 30 mM phosphate, 10 mM histidine, | 2.2 | 9.3 | 21.8 |
| 40 mg/mL mannitol, 0.01% (m/v) polysorbate 80, 0.5 ppm | | | |
| iron | | | |
| pH 6, 100 mg/mL J695, 30 mM phosphate, 10 mM | 2.0 | 4.4 | 12.6 |
| histidine, 40 mg/mL mannitol, 0.01% (m/v) polysorbate 80 | | | |
| pH 6, 100 mg/mL J695, 30 mM phosphate, 10 mM | 2.1 | 4.8 | 13.6 |
| histidine, 40 mg/mL mannitol, 0.01% (m/v) polysorbate | | | |
| 80, 0.5 ppm iron | | | |

### Example 9.7

### Fragmentation of J695 Formulated in Acetate Buffer in the Presence of Iron and Histidine at Solution pH 6

Antibody J695 was formulated at 2 mg/mL, pH 6.0 in the following compositions:
a) 30 mM acetate, 10 mM histidine, 40 mg/mL mannitol, 0.01% (m/v) polysorbate 80; and
b) 30 mM acetate, 10 mM histidine, 40 mg/mL mannitol, 0.01% (m/v) polysorbate 80 and 0.5 ppm iron.
   Additionally, J695 was formulated at 100 mg/mL, pH 6.0 in the following compositions:
c) 30 mM acetate, 10 mM histidine, 40 mg/mL mannitol, 0.01% (m/v) polysorbate 80; and
d) 30 mM acetate, 10 mM histidine, 40 mg/mL mannitol, 0.01% (m/v) polysorbate 80, 0.5 ppm iron.

Incubation at various temperatures, sample pull, and analysis of fragmentation in the resulting four formulations was performed as outlined in Example 9.1.

The results of these studies are provided in Table 15.7. The results demonstrate that the presence of iron and histidine in J695 formulations results in J695 fragmentation as compared to the control (i.e., the formulation lacking iron) and that the presence of other organic based buffers, such as acetate, does not impact iron-histidine mediated fragmentation of J695, regardless of protein concentration (e.g., 2 mg/mL or 100 mg/mL).

TABLE 15.7
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 30 mM acetate, 10 mM histidine, 40 mg/mL | 2.4 | 5.7 | 17.2 |
| mannitol, 0.01% (m/v) polysorbate 80 | | | |
| pH 6, 2 mg/mL J695, 30 mM acetate, 10 mM histidine, 40 mg/mL | 2.4 | 12.5 | 26.4 |
| mannitol, 0.01% (m/v) polysorbate 80, 0.5 ppm | | | |
| iron | | | |
| pH 6, 100 mg/mL J695, 30 mM acetate, 10 mM histidine, | 1.9 | 5.1 | 14.7 |
| 40 mg/mL mannitol, 0.01% (m/v) polysorbate 80 | | | |
| pH 6, 100 mg/mL J695, 30 mM acetate, 10 mM histidine, | 2.0 | 7.6 | 20.7 |
| 40 mg/mL mannitol, 0.01% (m/v) polysorbate 80, 0.5 ppm | | | |
| iron | | | |

### Example 9.8

### Fragmentation of J695 in the Presence of Polysorbate 80

J695 was formulated at 2 mg/mL, pH 6 in the following compositions:
a) 10 mM methionine, 10 mM histidine, 40 mg/mL mannitol
b) 10 mM methionine, 10 mM histidine, 40 mg/mL mannitol, and 0.5 ppm iron
c) 10 mM methionine, 10 mM histidine, 40 mg/mL mannitol, 0.01% (m/v) polysorbate 80
d) 10 mM methionine, 10 mM histidine, 40 mg/mL mannitol, 0.01% (m/v) polysorbate 80, and 0.5 ppm iron
   Additionally, J695 was formulated at 100 mg/mL, pH 6.0 in the compositions (a) to (d) as listed above. The results of this experiment are provided in Table 15.9 and are discussed below in Example 9.9.

### Example 9.9

### Fragmentation of J695 in the Presence of Poloxamer 188

J695 was formulated at 2 mg/mL, pH 6.0 in the following compositions:
a) 10 mM methionine, 10 mM histidine, 40 mg/mL mannitol;
b) 10 mM methionine, 10 mM histidine, 40 mg/mL mannitol, and 0.5 ppm iron;
c) 10 mM methionine, 10 mM histidine, 40 mg/mL mannitol, 0.1% (m/v) poloxamer 188; and
d) 10 mM methionine, 10 mM histidine, 40 mg/mL mannitol, 0.1% (m/v) poloxamer 188, and 0.5 ppm iron.
   Additionally, J695 was formulated at 100 mg/mL, pH 6.0, in the compositions (a) to (d) as listed above

The results of the experiments described in examples 9.8 and 9.9 are provided in Table 15.9. The results demonstrate that the presence of iron and histidine in J695 formulations results in J695 fragmentation as compared to the control (i.e., the formulation lacking iron) and that the presence or absence of surfactants, such as polysorbate 80 or poloxamer 188, does not impact iron-histidine mediated fragmentation of J695, regardless of protein concentration (e.g., 2 mg/mL or 100 mg/mL) and surfactant type and concentration.

TABLE 15.9
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 10 mM methionine, 10 mM | 2.1 | 4.8 | 13.4 |
| histidine, 40 mg/mL mannitol | | | |
| pH 6, 2 mg/mL J695, 10 mM 10 mM methionine, 10 mM | 2.1 | 9.7 | 26.2 |
| histidine, 40 mg/mL mannitol, 0.5 ppm iron | | | |
| pH 6, 100 mg/mL J695, 10 mM 10 mM methionine, 10 mM | 1.9 | 5.2 | 14.4 |
| histidine, 40 mg/mL mannitol | | | |
| pH 6, 100 mg/mL J695, 10 mM 10 mM methionine, 10 mM | 2.3 | 7.8 | 21.7 |
| histidine, 40 mg/mL mannitol, 0.5 ppm iron | | | |
| pH 6, 2 mg/mL J695, 10 mM 10 mM methionine e, 10 mM | 2.0 | 4.7 | 13.0 |
| histidine, 40 mg/mL mannitol, 1 mg/mL poloxamer 188 | | | |
| pH 6, 2 mg/mL J695, 10 mM 10 mM methionine 10 mM | 2.1 | 9.0 | 24.4 |
| histidine, 40 mg/mL mannitol, 0.5 ppm iron, 1 mg/mL | | | |
| poloxamer 188 | | | |
| pH 6, 100 mg/mL J695, 10 mM 10 mM methionine, 10 mM | 2.0 | 5.1 | 15.0 |
| histidine, 40 mg/mL mannitol, 1 mg/mL poloxamer | | | |
| 188 | | | |
| pH 6, 100 mg/mL J695, 10 mM 10 mM methionine, 10 mM | 2.1 | 8.0 | 21.4 |
| histidine, 40 mg/mL mannitol, 0.5 ppm iron, 1 mg/mL | | | |
| poloxamer 188 | | | |

### Example 9.10

### Fragmentation of J695 in the Presence of Mannitol

J695 was formulated at 2 mg/mL, pH 6.0 in the following compositions:
a) 10 mM methionine, 10 mM histidine, 0.01% (m/v) polysorbate 80;
b) 10 mM methionine, 10 mM histidine, 0.01% (m/v) polysorbate 80, and 0.5 ppm iron;
c) 10 mM methionine, 10 mM histidine, 150 mg/mL mannitol, 0.01% (m/v) polysorbate 80; and
d) 10 mM methionine, 10 mM histidine, 150 mg/mL mannitol, 0.01% (m/v) polysorbate 80, and 0.5 ppm iron.
   Additionally, J695 was formulated at 100 mg/mL, pH 6.0 in the compositions (a) to (d) as listed above. Incubation at various temperatures, sample pull, and analysis of J695 fragmentation in the resulting eight formulations was performed as outlined in Example 9.1.

The results of these studies are listed in Table 15.10. The results demonstrate that the presence of iron and histidine in J695 formulations results in J695 fragmentation as compared to the control (i.e., the formulation lacking iron) and that this fragmentation process is not impacted by various concentrations of sugars and sugar alcohols such as mannitol (e.g., 0 and 150 mg/mL), and protein concentration (e.g., 2 mg/mL and 100 mg/mL J695).

TABLE 15.10
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 10 mM methionine, 10 mM | 2.1 | 4.9 | 15.8 |
| histidine, 0 mg/mL mannitol, 0.1 mg/mL polysorbate 80 | | | |
| pH 6, 2 mg/mL J695, 10 mM methionine, 10 mM | 2.0 | 13.9 | 30.0 |
| histidine, 0 mg/mL mannitol, 0.1 mg/mL polysorbate 80, | | | |
| 0.5 ppm iron | | | |
| pH 6, 2 mg/mL J695, 10 mM methionine, 10 mM | 2.0 | 4.6 | 14.0 |
| histidine, 150 mg/mL mannitol, 0.1 mg/mL polysorbate 80 | | | |
| pH 6, 2 mg/mL J695, 10 mM methionine, 10 mM | 2.0 | 13.5 | 29.1 |
| histidine, 150 mg/mL mannitol, 0.1 mg/mL polysorbate | | | |
| 80, 0.5 ppm iron | | | |
| pH 6, 100 mg/mL J695, 10 mM methionine, 10 mM | 1.8 | 5.1 | 14.7 |
| histidine, 0 mg/mL mannitol, 0.1 mg/mL polysorbate 80 | | | |
| pH 6, 100 mg/mL J695, 10 mM methionine, 10 mM | 1.8 | 8.2 | 22.0 |
| histidine, 0 mg/mL mannitol, 0.1 mg/mL polysorbate 80, | | | |
| 0.5 ppm iron | | | |
| pH 6, 100 mg/mL J695, 10 mM methionine, 10 mM | 1.8 | 4.4 | 12.8 |
| histidine, 150 mg/mL mannitol, 0.1 mg/mL polysorbate 80 | | | |
| pH 6, 100 mg/mL J695, 10 mM methionine, 10 mM | 1.8 | 7.6 | 21.5 |
| histidine, 150 mg/mL mannitol, 0.1 mg/mL polysorbate | | | |
| 80, 0.5 ppm iron | | | |

### Example 9.11

### Fragmentation of J695 in the Presence of Desferrioxamine

J695 was formulated at 2 mg/mL, pH 6.0 in the following compositions:
a) 10 mM methionine, 10 mM histidine, 40 mg/mL mannitol, 0.01% (m/v) polysorbate 80;
b) 10 mM methionine, 10 mM histidine, 40 mg/mL mannitol, 0.01% (m/v) polysorbate 80 and 0.5 and 2.5 ppm iron;
c) 10 mM methionine, 10 mM histidine, 40 mg/mL mannitol, 0.01% (m/v) polysorbate 80 and 1 mM desferrioxamine; and
d) 10 mM methionine, 10 mM histidine, 40 mg/mL mannitol, 0.01% (m/v) polysorbate 80, 0.5 and 2.5 ppm iron and 1 mM desferrioxamine.
   Additionally, J695 was formulated at 100 mg/mL, pH 6.0 in the compositions (a) to (d) as listed above. Incubation at various temperatures, sample pull, and analysis of J695 fragmentation in the resulting twelve formulations was performed as outlined in Example 9.1.

The key results of these studies are listed in Table 15.11. The results demonstrate that the presence of desferrioxamine in J695 formulations does not negatively impact the stability of J695 as compared to the control (i.e., the formulation lacking desferrioxamine). The results further demonstrate that desferrioxamine reduces the histidine iron mediated fragmentation in J695 formulation over a broad iron concentration and over a broad protein concentration (e.g., at 2 mg/mL and 100 mg/mL J695).

TABLE 15.11
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 10 mM methionine, 10 mM | 1.8 | 4.7 | 12.0 |
| histidine, 40 mg/mL mannitol, 0.1 mg/mL polysorbate 80, | | | |
| 1 mM desferrioxamine | | | |
| pH 6, 2 mg/mL J695, 10 mM methionine, 10 mM | 1.9 | 4.7 | 12.2 |
| histidine, 40 mg/mL mannitol, 0.1 mg/mL polysorbate 80, | | | |
| 1 mM desferrioxamine, 0.5 ppm iron | | | |
| pH 6, 2 mg/mL J695, 10 mM methionine, 10 mM | 1.8 | 4.6 | 12.3 |
| histidine, 40 mg/mL mannitol, 0.1 mg/mL polysorbate 80, | | | |
| 1 mM desferrioxamine, 2.5 ppm iron | | | |
| pH 6, 100 mg/mL J695, 10 mM methionine, 10 mM | 1.8 | 4.7 | 12.3 |
| histidine, 40 mg/mL mannitol, 0.1 mg/mL polysorbate 80, | | | |
| 1 mM desferrioxamine | | | |
| pH 6, 100 mg/mL J695, 10 mM methionine, 10 mM | 1.6 | 4.9 | 12.4 |
| histidine, 40 mg/mL mannitol, 0.1 mg/mL polysorbate 80, | | | |
| 1 mM desferrioxamine, 0.5 ppm iron | | | |
| pH 6, 100 mg/mL J695, 10 mM methionine, 10 mM | 1.7 | 4.9 | 12.6 |
| histidine, 40 mg/mL mannitol, 0.1 mg/mL polysorbate 80, | | | |
| 1 mM desferrioxamine, 2.5 ppm iron | | | |

### Example 9.12

### Fragmentation of J695 in the Presence of a Citrate Salt

J695 was formulated at 2 mg/mL, pH 6.0 in the following compositions:
a) 10 mM methionine, 10 mM histidine, 40 mg/mL mannitol, 0.01% (m/v) polysorbate 80;
b) 10 mM methionine, 10 mM histidine, 40 mg/mL mannitol, 0.01% (m/v) polysorbate 80 and 0.5 ppm iron;
c) 10 mM methionine, 10 mM histidine, 40 mg/mL mannitol, 0.01% (m/v) polysorbate 80 and 30 mM citrate; and
d) 10 mM methionine, 10 mM histidine, 40 mg/mL mannitol, 0.01% (m/v) polysorbate 80, 0.5 ppm iron and 30 mM citrate.

Additionally, J695 was formulated at 100 mg/mL, pH 6.0 in the compositions (a) to (d) as listed above. Incubation at various temperatures, sample pull and analysis of J695 fragmentation in the resulting eight formulations was performed as outlined in Example 9.1.

The key results of these studies are listed in Table 15.12. The results demonstrate that the presence of citrate in the J695 formulations does not negatively impact stability of J695 compared to the control (i.e., the formulation lacking citrate). The results further demonstrate that citrate reduces the histidine iron mediated fragmentation in J695 formulations over a broad protein concentration (2 mg/mL and 100 mg/mL J695).

TABLE 15.12
Fragment levels of J695 with different formulations during accelerated
stability studies.

| | | T1 month, | T3 months, |
|---|---|---|---|
| Formulation composition | T0, 5° C. | 40° C. | 40° C. |
| pH 6, 2 mg/mL J695, 10 mM methionine, 10 mM | 1.8 | 4.4 | 12.5 |
| histidine, 40 mg/mL mannitol, 0.1 mg/mL polysorbate 80, | | | |
| 30 mM citrate | | | |
| pH 6, 2 mg/mL J695, 10 mM methionine, 10 mM | 1.7 | 4.6 | 12.5 |
| histidine, 40 mg/mL mannitol, 0.1 mg/mL polysorbate 80, | | | |
| 30 mM citrate, 0.5 ppm iron | | | |
| pH 6, 100 mg/mL J695, 10 mM methionine, 10 mM | 1.6 | 4.6 | 13.1 |
| histidine, 40 mg/mL mannitol, 0.1 mg/mL polysorbate 80, | | | |
| 30 mM citrate | | | |
| pH 6, 100 mg/mL J695, 10 mM methionine, 10 mM | 1.7 | 4.9 | 13.0 |
| histidine, 40 mg/mL mannitol, 0.1 mg/mL polysorbate 80, | | | |
| 30 mM citrate, 0.5 ppm iron | | | |

### Example 9.13

### Fragmentation of J695 in the Presence of Desferrithiocin

J695 is formulated at 2 mg/mL, pH 6.0 in the following compositions:
a) 10 mM methionine, 10 mM histidine, 40 mg/mL mannitol, 0.01% (m/v) polysorbate 80;
b) 10 mM methionine, 10 mM histidine, 40 mg/mL mannitol, 0.01% (m/v) polysorbate 80 and 0.5 ppm iron;
c) 10 mM methionine, 10 mM histidine, 40 mg/mL mannitol, 0.01% (m/v) polysorbate 80 and 0.1 mM desferrithiocin; and
d) 10 mM methionine, 10 mM histidine, 40 mg/mL mannitol, 0.01% (m/v) polysorbate 80, 0.5 ppm iron and 0.1 mM desferrithiocin.

Additionally, J695 is formulated at 100 mg/mL, pH 6.0 in the compositions (a) to (d) as listed above. Incubation at various temperatures, sample pull, and analysis of J695 fragmentation in the resulting eight formulations is performed as outlined in Example 9.1.

### Example 9.14

### Mutations of Residues in the Hinge Region

J695 with specific residues mutated in the hinge region is formulated at 2 mg/mL, pH 6 in the following compositions:
a) 10 Mm methionine, 10 mM histidine, 40 mg/mL mannitol, 0.01% (m/v) polysorbate 80; and
b) 10 mM methionine, 10 mM histidine, 40 mg/mL mannitol, 0.01% (m/v) polysorbate 80 and 0.5 ppm iron.

Additionally, J695 is formulated at 100 mg/mL in the compositions (a) to (b) as listed above. Incubation at various temperatures, sample pull, and analysis of J695 fragmentation in the resulting four formulations is performed as outlined in Example 9.1.

### Example 9.15

### Removal of Histidine from the Formulation

J695 was formulated at 17 mg/mL, pH 6.0 in the following compositions:
a) 10 mM methionine, 10 mM imidazole, 40 mg/mL mannitol; and
b) 10 mM methionine, 10 mM imidazole, 40 mg/mL mannitol and 100 ppm iron (II) sulfate.

Incubation of the compositions was performed at 40° C. for 2 weeks and analysis was carried out by non-reducing CE-SDS as described in Example 7.2.4.

The key results of these studies are listed in Table 15.13 below. The results demonstrate that removal of histidine and replacement with imidazole does not result in fragmentation of J695 in the presence of iron. These results demonstrate that removal of histidine inhibits or prevents fragmentation of J695 in the presence of iron.

TABLE 15.13
Fragment levels of J695 without histidine in formulation and after
accelerated stability studies. J695 was formulated at 17 mg/mL, pH 6.0 as specified in a)
and b) above.

| | LC & others | HC/FAB | (HCLC) | Fragment 2 | (2)HC(1)LC | intact |
|---|---|---|---|---|---|---|
| 10 mM methionine, 10 mM | 1.16 | 0.46 | 0.21 | 1.29 | 3.24 | 93.65 |
| imidazole, 40 mg/mL | | | | | | |
| mannitol | | | | | | |
| 10 mM methionine, 10 mM | 0.95 | 0.49 | 0.42 | 1.25 | 3.14 | 93.76 |
| imidazole, 40 mg/mL | | | | | | |
| mannitol and 100 ppm iron | | | | | | |
| (II) sulfate | | | | | | |

### Example 9.16

### Removal of Iron Via Ultrafiltration/Diafiltration or by Dialysis

J695 containing iron (500 ppm) and J695 without iron (60 ppm) as a control were dialyzed into either formulation buffer (10 mM histidine, 10 mM methionine, 4% mannitol, pH 6.0) or citrate/phosphate buffer (10 mM sodium hydrogen phosphate, 10 mM citric acid; pH=6.0). The samples were then incubated for 1 month at 40° C. The samples were analyzed by non-reducing CE-SDS following incubation to determine the amount of fragment present.

The results are provided in Table 15.14 below. The results demonstrate that dialysis against formulation buffer or citrate/phosphate buffer results in a reduction in fragmentation. Dialysis into the citrate/phosphate buffer resulted in a greater decrease in fragmentation as compared to dialysis against formulation buffer, indicating a possible role of citrate/phosphate in binding iron and stripping iron from protein.

TABLE 15.14
Fragment levels of J695 after dialysis and accelerated stability studies.

| | LC & others | HC/FAB | (HCLC) | Fragment 2 | (2)HC(1)LC | intact |
|---|---|---|---|---|---|---|
| J695 with 60 ppm of iron after 1 month | 0.62 | 0.6 | 0.23 | 1.51 | 2.55 | 94.48 |
| 40° C. | | | | | | |
| J695 with 500 ppm of iron after 1 month | 1.65 | 0.91 | 0.3 | 3.41 | 3.97 | 89.66 |
| 40° C. | | | | | | |
| J695 with 500 ppm iron and dialysis with | 0.57 | 0.6 | 0.21 | 1.45 | 2.69 | 94.48 |
| citrate buffer after 1 month 40° C. | | | | | | |
| J695 with 500 ppm iron and dialysis with | 1.02 | 0.78 | 0.22 | 2.1 | 3.21 | 92.66 |
| formulation buffer after 1 month 40° C. | | | | | | |

### Example 10

### Fragmentation Of J695 at Various Levels of Iron and at Different Temperatures

Analysis by SEC showed enhanced fragmentation in J695 at 25° C. and at 40° C. with increasing iron levels. No impact of iron, spiked up to 10,000 ppb, was observed after 6 months of storage at 5° C.

### Example 10.1

### Fragmentation of J695 (100 mg/mL) at Various Levels of Iron and at Different Temperatures

After the formulation buffer for the J695 antibody was selected, the drug substance was formulated in the same matrix as the finished product. The main goal of protein formulation is to maintain the stability of a given protein in its native, pharmaceutically active form over prolonged periods of time to guarantee an acceptable shelf-life of the pharmaceutical protein drug. The recommended storage temperature for the J695 pre-filled syringe (PFS) is from 2-8° C. and the normal iron levels measured in various lots of J695 was about 60 ppb (Table 16). The impact of spiking different levels of iron on fragmentation after storing the PFS at the recommended storage temperature of 5° and at elevated temperatures of 25° C. and 40° C. for up to 6 months was assessed.

The antibody, J695, was formulated at 100 mg/mL in a pre-filled syringe (PFS), maintained at pH 6.0 in the following nominal compositions:
1. 10 mM methionine, 10 mM histidine, 40 mg/mL mannitol, 0.01% (m/v) polysorbate 80
2. 10 mM methionine, 10 mM histidine, 40 mg/mL mannitol, 0.01% (m/v) polysorbate 80 and 10 ppb iron as Fe (II) sulfate
3. 10 mM methionine, 10 mM histidine, 40 mg/mL mannitol, 0.01% (m/v) polysorbate 80 and 50 ppb iron as Fe (II) sulfate
4. 10 mM methionine, 10 mM histidine, 40 mg/mL mannitol, 0.01% (m/v) polysorbate 80 and 100 ppb iron as Fe (II) sulfate
5. 10 mM methionine, 10 mM histidine, 40 mg/mL mannitol, 0.01% (m/v) polysorbate 80 and 250 ppb iron as Fe (II) sulfate
6. 10 mM methionine, 10 mM histidine, 40 mg/mL mannitol, 0.01% (m/v) polysorbate 80 and 500 ppb iron as Fe (II) sulfate
7. 10 mM methionine, 10 mM histidine, 40 mg/mL mannitol, 0.01% (m/v) polysorbate 80 and 1 ppm iron as Fe (II) sulfate
8. 10 mM methionine, 10 mM histidine, 40 mg/mL mannitol, 0.01% (m/v) polysorbate 80 and 5 ppm iron as Fe (II) sulfate
9. 10 mM methionine, 10 mM histidine, 40 mg/mL mannitol, 0.01% (m/v) polysorbate 80 and 10 ppm iron as Fe (II) sulfate

The resulting formulations were filled into pre-filled syringes (PFS) and incubated at 5, 25 and 40° C. for up to 6 months. At predetermined points of time, samples of all formulations were pulled, and the extent of antibody fragmentation in the various formulations was determined by SEC. As seen in Table 16, there was no impact of iron (spiked up to 10,000 ppb) on fragmentation observed after 6 months at the recommended storage conditions. These studies indicate that at the recommended storage conditions the J695 formulation maintained the stability of a given protein in its native, pharmaceutically active form over prolonged periods of time to provide an acceptable shelf-life of the pharmaceutical protein drug.

The impact on fragmentation at elevated temperature by spiking different levels of iron into J695 pre-filled syringes and storing at 25° and 40° C. was also evaluated. As seen in Table 16, spiking iron above about 160 ppb (corresponding to 60 ppb normal Fe level+100 ppb added for the spiking experiment) lead to increased fragmentation at 25° C. and 40° C. as assessed by SEC.

TABLE 16

| storage in | | Temperature | | |
|---|---|---|---|---|
| months | characteristic | 5° C. | 25° C. | 40° C. |
| spike 10 ppb | | | | |
| 0 | fragments | 1.7 | n/a | n/a |
| 1 | fragments | 1.4 | 1.6 | 3.6 |
| 3 | fragments | 1.4 | 2.2 | 7.7 |
| 6 | fragments | 1.2 | 3.1 | 13.2 |
| spike 50 ppb | | | | |
| 0 | fragments | 1.8 | n/a | n/a |
| 1 | fragments | 1.3 | 1.6 | 4.1 |
| 3 | fragments | 1.4 | 2.4 | 8.8 |
| 6 | fragments | 1.3 | 3.4 | 14.6 |
| spike 100 ppb | | | | |
| 0 | fragments | 1.7 | n/a | n/a |
| 1 | fragments | 1.3 | 1.5 | 4.7 |
| 3 | fragments | 1.4 | 2.6 | 10.2 |
| 6 | fragments | 1.4 | 3.8 | 16.3 |
| spike 250 ppb | | | | |
| 0 | fragments | 1.6 | n/a | n/a |
| 1 | fragments | 1.2 | 1.8 | 6.7 |
| 3 | fragments | 1.4 | 3.6 | 13.9 |
| 6 | fragments | 1.4 | 5.3 | 20.5 |
| spike 500 ppb | | | | |
| 0 | fragments | 1.7 | n/a | n/a |
| 1 | fragments | 1.1 | 2.2 | 8.4 |
| 3 | fragments | 1.5 | 4.7 | 17.6 |
| 6 | fragments | 1.7 | 7.5 | 25.0 |
| spike 1,000 ppb | | | | |
| 0 | fragments | 1.7 | n/a | n/a |
| 1 | fragments | 2.7 | 3.8 | 12.0 |
| 3 | fragments | 1.2 | 4.5 | 19.1 |
| 6 | fragments | 1.3 | 8.2 | 27.8 |
| spike 5,000 ppb | | | | |
| 0 | fragments | 1.9 | n/a | n/a |
| 1 | fragments | 4.3 | 4.6 | 13.5 |
| 3 | fragments | 1.2 | 5.4 | 21.2 |
| 6 | fragments | 1.5 | 10.1 | 30.9 |
| spike 10,000 ppb | | | | |
| 0 | fragments | 1.9 | n/a | n/a |
| 1 | fragments | 4.2 | 4.8 | 13.3 |
| 3 | fragments | 1.3 | 5.7 | 21.3 |
| 6 | fragments | 1.6 | 10.3 | 30.0 |

### Example 10.2

### Fragmentation of J695 (2 mg/mL) at Various Levels of Iron and at Different Temperatures

Additionally, J695 is formulated at 2 mg/mL, pH 6.0 in the nominal compositions (1) to (9) as listed above. The resulting 9 formulations are filled into sterile, non-pyrogenic polypropylene cryogenic vials and incubated at 5°, 25° and 40° C. for up to 6 months. Additionally, all 9 formulations are stored at the recommended storage temperature at 2-8° C. for up to 12 months. At pre-determined points of time, samples of all formulations are pulled, and the extent of antibody fragmentation in the various formulations is determined by SEC.

### INCORPORATION BY REFERENCE

The contents of all cited references (including literature references, patents, patent applications, and websites) that maybe cited throughout this application are hereby expressly incorporated by reference in their entirety, as are the references cited therein. The practice of the present invention will employ, unless otherwise indicated, conventional techniques of protein formulation, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

We claim:

1. A method for inhibiting or preventing cleavage of a molecule comprising at least a portion of a lambda light chain in a histidine containing formulation, the method comprising the step of inhibiting or preventing the ability of metals to cleave the molecule.

2. The method of claim 1, wherein the inhibiting or preventing comprises including at least one metal chelator in the formulation.

3. The method of claim 1, wherein the inhibiting or preventing comprises subjecting the formulation to at least one procedure selected from the group consisting of filtration, buffer exchange, chromatography, and resin exchange.

4. The method of claim 3, wherein the filtration is selected from the group consisting of ultrafiltration and diafiltration.

5. The method of claim 3, wherein the buffer exchange comprises dialysis with a buffer selected from the group consisting of a buffer comprising histidine, a buffer comprising citrate and phosphate and a buffer comprising imidazole.

6. The method of claim 1, wherein the inhibiting or preventing comprises including a citrate buffer or a phosphate buffer in the formulation.

7. The method of claim 1, wherein the inhibiting or preventing comprises inhibiting or preventing cleavage by altering at least one amino acid in the lambda light chain.

8. The method of claim 1, wherein the inhibiting or preventing comprises inhibiting or preventing cleavage by altering the amino acid sequence in the lambda chain such that an amino acid sequence glutamic acid-cysteine-serine is changed.

9. The method of claim 1, wherein the formulation comprises about 10 mM histidine.

10. The method of claim 1, wherein the formulation comprises about 1-100 mM histidine.

11. The method of claim 1, wherein the cleavage occurs in a hinge region of the lambda chain.

12. The method of claim 1, wherein the at least a portion of a lambda light chain comprises the amino acid sequence of glutamic acid-cysteine-serine, or at least one modification that does not inhibit antibody binding.

13. The method of claim 12, wherein the cleavage occurs between the glutamic acid and the cysteine.

14. The method of claim 1, wherein the molecule comprises at least a portion of a heavy chain.

15. The method of claim 1, wherein the portion of a heavy chain comprises the amino acid sequence SCDK, or at least one modification that does not inhibit antibody binding.

16. The method of claim 10, wherein the cleavage occurs between the serine and the cysteine.

17. The method of claim 10, wherein the cleavage occurs between the cysteine and the aspartic acid.

18. The method of claim 1, wherein the metal is Fe2+.

19. The method of claim 1, wherein the metal is Fe3+.

20. The method of claim 1, wherein the metal is Cu2+.

21. The method of claim 1, wherein the metal is Cu1+.

22. The method of claim 1, wherein the molecule is present in a concentration range of about 1 mg/ml to about 300 mg/ml.

23. The method of claim 1, wherein the molecule is present in a concentration of about 2 mg/ml.

24. The method of claim 1, wherein the molecule is present in a concentration of about 7 mg/ml.

25. The method of claim 1, wherein the molecule is present in a concentration of about 100 mg/ml.

26. The method of claim 1, wherein the molecule is an immunoglobulin.

27. The method of claim 1, wherein the molecule is a monoclonal antibody.

28. The method of claim 1, wherein the molecule is selected from the group consisting of a DVD-Ig™, a Fab fragment, a F(ab′)₂ fragment, a chimeric antibody, a CDR-grafted antibody, a humanized antibody, a human antibody, a disulfide linked Fv, a single domain antibody, a multispecific antibody, a dual specific antibody, and a bispecific antibody.

29. The method of claim 1, wherein the molecule is an anti-IL-12/23 antibody.

30. The method of claim 1, wherein the molecule is J695.

31. The method of claim 1, wherein the molecule is an anti-CD80 or anti-IGF1,2 antibody.

32. The method of claim 1, wherein the cleavage occurs at a temperature of about 2° C. to about 25° C.

33. The method of claim 1, wherein the cleavage occurs at a temperature of about 2° C. to about 8° C.

34. The method of claim 1, wherein the cleavage occurs at a pH of about 4 to about 8.

35. The method of claim 1, wherein the cleavage occurs at a pH of about 5 to about 6.

36. The method of claim 1, wherein the inhibiting or preventing comprises lowering the pH to about 5 or less.

37. The method of claim 2, wherein the at least one metal chelator is selected from the group consisting of citrate, a siderophore, calixerenes, an aminopolycarboxylic acid, a hydroxyaminocarboxylic acid, an N-substituted glycine, a 2-(2-amino-2-oxoethyl)aminoethane sulfonic acid (BES), a bidentate, tridentate or hexadentate iron chelator, and derivatives, analogues, and combinations thereof.

38. The method of claim 2, wherein the at least one metal chelator is a siderophore selected from the group consisting of aerobactin, agrobactin, azotobactin, bacillibactin, N-(5-C3-L (5 aminopentyl)hydroxycarbamoyl)-propionamido)pentyl)-3(5-(N-hydroxyacetoamido)-pentyl)carbamoyl)-proprionhydroxamic acid (deferoxamine, desferrioxamine or DFO or DEF), desferrithiocin, enterobactin, erythrobactin, ferrichrome, ferrioxamine B, ferrioxamine E, fluviabactin, fusarinine C, mycobactin, parabactin, pseudobactin, vibriobactin, vulnibactin, yersiniabactin, ornibactin and derivatives, analogues, and combinations thereof.

39. The method of claim 38, wherein the metal chelator is desferrioxamine.

40. The method of claim 2, wherein the at least one metal chelator is citrate.

41. The method of claim 2, wherein the at least one metal chelator is an aminopolycarboxylic acid selected from the group consisting of ethylenediaminetetraacetic acid (EDTA), nitriloacetic acid (NTA), trans-diaminocyclohexane tetraacetic acid (DCTA), diethylenetriamine pentaacetic acid (DTPA), N-2-acetamido-2-iminodiacetic acid (ADA), aspartic acid, bis(aminoethyl)glycolether N,N,N′N′-tetraacetic acid (EGTA), glutamic acid, and N,N′-bis(2-hydroxybenzyl)ethylenediamine-N,N′-diacetic acid (HBED), and derivatives, analogues, and combinations thereof.

42. The method of claim 2, wherein the at least one metal chelator is a hydroxyaminocarboxylic acid selected from the group consisting of N-hydroxyethyliminodiacetic acid (HIMDA), N,N-bishydroxyethylglycine (bicine), and N-(trishydroxymethylmethyl)glycine (tricine), and derivatives, analogues, and combinations thereof.

43. The method of claim 2, wherein the at least one metal chelator is an N-substituted glycine, or derivative, analogue, or combination thereof.

44. The method of claim 43, wherein the N-substituted glycine is selected from the group consisting of glycylglycine, and derivatives, analogues, and combinations thereof.

45. The method of claim 2, wherein the at least one metal chelator is 2-(2-amino-2-oxoethyl)aminoethane sulfonic acid (BES), and derivatives, analogues, and combinations thereof.

46. The method of claim 2, wherein the at least one metal chelator comprises a combination of DTPA and DEF.

47. The method of claim 2, wherein the at least one metal chelator comprises a combination of EDTA, EGTA and DEF.

48. The method of claim 2, wherein the at least one metal chelator is a calixarene, selected from the group consisting of a macrocycle or cyclic oligomer based on a hydroxyalkylation product of a phenol and an aldehyde, and derivatives, analogues, and combinations thereof.

49. The method of claim 2, wherein the at least one metal chelator is a hydroxypyridine-derivate, a hydrazone-derivate, and hydroxyphenyl-derivate, or a nicotinyl-derivate, such as 1,2-dimethyl-3-hydroxypyridin-4-one (Deferiprone, DFP or Ferriprox); 2-deoxy-2-(N-carbamoylmethyl-[N′-2′-methyl-3′-hydroxypyridin-4′-one])-D-glucopyranose (Feralex-G), pyridoxal isonicotinyl hydrazone (PIH); 4,5-dihydro-2-(2,4-dihydroxyphenyl)-4-methylthiazole-4-carboxylic acid (GT56-252), 4-[3,5-bis(2-hydroxyphenyl)-[1,2,4]triazol-1-yl]benzoic acid (ICL-670); N,N′-bis(o-hydroxybenzyl)ethylenediamine-N,N′-diacetic acid (HBED), 5-chloro-7-iodo-quinolin-8-ol (clioquinol), and derivatives, analogues, and combinations thereof.

50. The method of claim 2, wherein the at least one metal chelator is a copper chelator selected from the group consisting of triethylenetetramine (trientine), etraethylenepentamine, D-penicillamine, ethylenediamine, bispyridine, phenantroline, bathophenanthroline, neocuproine, bathocuproine sulphonate, cuprizone, cis,cis-1,3,5,-triaminocyclohexane (TACH), tachpyr, and derivatives, analogues, and combinations thereof.

51. The method of claim 1, wherein the formulation comprises at least one additional excipient selected from the group consisting of amino acid, sugar, sugar alcohol, buffer, salt, and surfactant.

52. The method of claim 1, wherein the formulation comprises at least one additional excipient selected from the group consisting of about 1 to 60 mg/ml mannitol, about 1 to about 50 mM methionine, about 0.001% to about 0.5% (w/v) polysorbate 80, about 0.001% to about 1% (w/v) polyoxamer 188, about 1 to about 150 mM sodium chloride, about 1 to about 30 mM acetate, about 1 to about 30 mM citrate, about 1 to about 30 mM phosphate, and about 1 to about 30 mM arginine.

53. A method for detecting cleavage of a molecule comprising at least a portion of a lambda light chain in a histidine containing formulation, the method comprising the steps of including at least one metal chelator in the formulation and analyzing the at least a portion of the molecule for cleavage.

54. A stable formulation comprising a molecule comprising at least a portion of a lambda light chain and a buffer system comprising histidine, wherein said formulation is substantially free of metal.

55. The formulation of claim 54, wherein the metal is Fe2+ or Fe3+.

56. The formulation of claim 54, wherein the metal is Cu2+ or Cu1+.

57. The formulation of claim 54, wherein the formulation is substantially free of metal following subjection to at least one procedure selected from the group consisting of filtration, buffer exchange, chromatography and resin exchange.

58. The formulation of claim 57, wherein the buffer exchange comprises dialysis with a buffer selected from the group consisting of a buffer comprising histidine, a buffer comprising citrate and phosphate and a buffer comprising imidazole.

59. The formulation of claim 54, wherein the metal is present at a concentration selected from the group consisting of less than about 5,060 ppb, less than about 1,060 ppb, less than about 560 ppb, less than about 310 ppb, less than about 160 ppb, less than about 110 ppb and less than about 70 ppb.

60. The formulation of claim 59, wherein the metal is present at a concentration of less than about 160 ppb.

61. The formulation of claim 59, wherein the metal is present at a concentration of less than about 70 ppb.

62. The formulation of claim 54, further comprising at least one additional excipient selected from the group consisting of a polyol and a surfactant.

63. The formulation of claim 62, further comprising a stabilizer.

64. The formulation of claim 54, further comprising mannitol, polysorbate 80 and methionine.

65. The formulation of claim 54, wherein the buffer system further comprises citrate or phosphate.

66. The formulation of claim 54, wherein the pH is about 5 or less.

67. The formulation of claim 64, which comprises
(a) 1-10% mannitol,
(b) 0.001-0.1% polysorbate-80,
(c) a buffer system comprising 1-100 mM histidine and 1-50 mM methionine, with a pH of 5 to 7.

68. The formulation of claim 64, which comprises
(a) 2-6% mannitol,
(b) 0.005-0.05% polysorbate-80,
(c) a buffer system comprising 5-50 mM histidine and 5-20 mM methionine, with a pH of 5 to 7.

69. The formulation of claim 64, which comprises
(a) about 4% mannitol,
(b) about 0.01% polysorbate-80,
(c) a buffer system comprising about 10 mM histidine and about 10 mM methionine, with a pH of about 6.

70. The formulation of any one of claims 54-69, wherein the molecule is a monoclonal antibody, or antigen binding portion thereof.

71. The formulation of claim 70, wherein the concentration of the antibody, or antigen binding portion thereof, is between about 1 and about 250 mg/ml.

72. The formulation of claim 70, wherein the concentration of the antibody, or antigen binding portion thereof, is between about 40 and about 200 mg/ml.

73. The formulation of claim 70, wherein the concentration of the antibody, or antigen binding portion thereof, is about 100 mg/ml.

74. The formulation of claim 70, wherein the antibody is a human antibody, or antigen binding portion thereof, capable of binding to an epitope of a p40 subunit of IL-12/IL-23.

75. The formulation of claim 74, wherein the human antibody is the antibody J695, or an antigen binding portion thereof.

76. The formulation of claim 74 or 75, which has a shelf life of at least 24 months.

77. The formulation of claim 74 or 75, which maintains stability following at least 5 freeze/thaw cycles of the formulation.

78. The formulation of claim 74 or 75, further comprising an additional agent.

79. The formulation of claim 78, wherein the additional agent is a therapeutic agent.

80. The formulation of claim 79, wherein the therapeutic agent is selected from the group consisting of budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β monoclonal antibody, an anti-IL-6 monoclonal antibody, a growth factor, an elastase inhibitor, a pyridinyl-imidazole compound, an antibody or agonist of TNF, LT, IL-1, IL-2, IL-6, IL-7, IL-8, IL-15, IL-16, IL-18, EMAP-II, GM-CSF, FGF, and PDGF, an antibody to CD2, CD3, CD4, CD8, CD25, CD28, CD30, CD40, CD45, CD69, CD90 or ligand thereof, methotrexate, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα converting enzyme inhibitor, a T-cell signalling inhibitor, a metalloproteinase inhibitor, an angiotensin converting enzyme inhibitor, a soluble cytokine receptor, soluble p55 TNF receptor, soluble p75 TNF receptor, sIL-1RI, sIL-1RII, sIL-6R, an antiinflammatory cytokine, IL-4, IL-10, IL-11, IL-13, and TGFβ.

81. The formulation of claim 79, wherein the therapeutic agent is selected from the group consisting of an anti-TNF antibody and antibody fragments thereof, a TNFR-Ig construct, a TACE inhibitor, a PDE4 inhibitor, a corticosteroid, budenoside, dexamethasone, sulfasalazine, 5-aminosalicylic acid, olsalazine, an IL-1β converting enzyme inhibitor, IL-1ra, a tyrosine kinase inhibitor, a 6-mercaptopurine, and IL-11.

82. The formulation of claim 79, wherein the therapeutic agent is selected from the group consisting of methylprednisolone, cyclophosphamide, 4-aminopyridine, tizanidine, interferon-β1a, interferon-β1b, Copolymer 1, hyperbaric oxygen, intravenous immunoglobulin, clabribine, a TACE inhibitor, a kinase inhibitor, sIL-13R, an anti-P7, and p-selectin glycoprotein ligand (PSGL).

83. A stable formulation comprising a molecule comprising at least a portion of a lambda light chain, a buffer system comprising imidazole, and a metal, wherein the molecule is not cleaved within the hinge region in the presence of a metal.

84. A stable formulation comprising a molecule comprising at least a portion of a lambda light chain, a buffer system comprising histidine, and a metal chelator, wherein the molecule is not cleaved within the hinge region or is cleaved within the hinge region at a level which is less than the level of cleavage observed in the absence of the metal chelator.

85. The formulation of claim 83 or 84, wherein the metal Fe2+ or Fe3+.

86. The formulation of claim 83 or 84, wherein the metal is Cu2+ or Cu1+.

87. The formulation of claim 84, wherein the metal chelator is selected from the group consisting of a siderophore, calixerenes, an aminopolycarboxylic acid, a hydroxyaminocarboxylic acid, an N-substituted glycine, a 2-(2-amino-2-oxoethyl)aminoethane sulfonic acid (BES), a bidentate, tridentate or hexadentate iron chelator, a copper chelator, citrate and derivatives, analogues, and combinations thereof.

88. The formulation of claim 87, wherein the metal chelator is desferrioxamine.

89. The formulation of claim 83 or 84, further comprising at least one additional excipient selected from the group consisting of a polyol and a surfactant.

90. The formulation of claim 89, further comprising a stabilizer.

91. The formulation of claim 84, further comprising mannitol, polysorbate 80 and methionine.

92. The formulation of claim 84, wherein the buffer system further comprises citrate or phosphate.

93. The formulation of claim 83 or 84, wherein the pH of the formulation is about 5 or less.

94. The formulation of claim 91, which comprises
(a) 1-10% mannitol,
(b) 0.001%-0.1% polysorbate-80,
(c) a buffer system comprising 1-100 mM histidine and 1-50 mM methionine, with a pH of 5 to 7.

95. The formulation of claim 91, which comprises
(a) 2-6% mannitol,
(b) 0.005-0.05% polysorbate-80,
(c) a buffer system comprising 5-50 mM histidine and 5-20 mM methionine, with a pH of 5 to 7.

96. The formulation of claim 91, which comprises
(a) about 4% mannitol,
(b) about 0.01% polysorbate-80,
(c) a buffer system comprising about 10 mM histidine and about 10 mM methionine, with a pH of about 6.

97. The formulation of any one of claims 84-96, wherein the molecule is a monoclonal antibody, or antigen binding portion thereof.

98. The formulation of claim 97, wherein the concentration of the antibody, or antigen binding portion thereof, is between about 1 and about 250 mg/ml.

99. The formulation of claim 97, wherein the concentration of the antibody, or antigen binding portion thereof, is between about 40 and about 200 mg/ml.

100. The formulation of claim 97, wherein the concentration of the antibody, or antigen binding portion thereof, is about 100 mg/ml.

101. The formulation of claim 97, wherein the antibody is a human antibody, or antigen binding portion thereof, capable of binding to an epitope of a p40 subunit of IL-12/IL-23.

102. The formulation of claim 101, wherein the human antibody is the antibody J695, or an antigen binding portion thereof.

103. The formulation of claim 101 or 102, which has a shelf life of at least 24 months.

104. The formulation of claim 101 or 102, which maintains stability following at least 5 freeze/thaw cycles of the formulation.

105. The formulation of claim 101 or 102, further comprising an additional agent.

106. The formulation of claim 105, wherein the additional agent is a therapeutic agent.

107. The formulation of claim 106, wherein the therapeutic agent is selected from the group consisting of budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β monoclonal antibody, IL-1 receptor antibody, an anti-IL-6 monoclonal antibody, an IL-6 receptor antibody, a growth factor, an elastase inhibitor, a pyridinyl-imidazole compound, an antibody or agonist of TNF, LT, IL-1, IL-2, IL-6, IL-7, IL-8, IL-15, IL-16, IL-17, IL-18, EMAP-II, GM-CSF, FGF, and PDGF, an antibody to CD2, CD3, CD4, CD8, CD20, CD25, CD28, CD30, CD40, CD45, CD69, CD90 or ligand thereof, methotrexate, FK506, rapamycin, mycophenolate mofetil, leflunomide, an NSAID, ibuprofen, prednisolone, a phosphodiesterase inhibitor, an adenosine agonist, an antithrombotic agent, a S1P1 agonist, a bcl-2 inhibitor, a complement inhibitor, an adrenergic agent, IRAK, NIK, IKK, p38, a MAP kinase inhibitor, an IL-1β converting enzyme inhibitor, a TNFα converting enzyme inhibitor, a T-cell signalling inhibitor, a metalloproteinase inhibitor, an angiotensin converting enzyme inhibitor, a soluble cytokine receptor, soluble p55 TNF receptor, soluble p75 TNF receptor, sIL-1RI, sIL-1RII, sIL-6R, an antiinflammatory cytokine, IL-4, IL-10, IL-11, IL-13, and TGFβ.

108. The formulation of claim 106, wherein the therapeutic agent is selected from the group consisting of an anti-TNF antibody and antibody fragments thereof, a TNFR-Ig construct, a TACE inhibitor, a PDE4 inhibitor, a corticosteroid, budenoside, dexamethasone, sulfasalazine, 5-aminosalicylic acid, olsalazine, an IL-1β converting enzyme inhibitor, IL-1ra, a tyrosine kinase inhibitor, a 6-mercaptopurine, and IL-11.

109. The formulation of claim 106, wherein the therapeutic agent is selected from the group consisting of methylprednisolone, cyclophosphamide, 4-aminopyridine, tizanidine, interferon-β1a, interferon-β1b, copolymer 1, hyperbaric oxygen, intravenous immunoglobulin, clabribine, a TACE inhibitor, a kinase inhibitor, sIL-13R, an anti-P7, and p-selectin glycoprotein ligand (PSGL).

110. A stable formulation comprising a therapeutically effective amount of an antibody comprising a lambda light chain in a buffered solution comprising histidine with a pH of about 5 to about 7, wherein metal is present in a concentration that does not result in cleavage of the lambda light chain in the presence of histidine.

111. The formulation of claim 110, wherein the cleavage occurs in a hinge region of the lambda chain.

112. The formulation of claim 110, wherein the metal is Fe2+ or Fe3+.

113. The formulation of claim 110, wherein the metal is Cu2+ or Cu1+.

114. The formulation of claim 110, wherein the metal is present at a concentration selected from the group consisting of less than about 5,060 ppb, less than about 1,060 ppb, less than about 560 ppb, less than about 310 ppb, less than about 160 ppb, less than about 110 ppb and less than about 70 ppb.

115. The formulation of claim 110, wherein the metal is present at a concentration of less than about 160 ppb.

116. The formulation of claim 110, wherein the metal is present at a concentration of less than about 70 ppb.

117. The formulation of claim 110, which comprises at least one additional excipient selected from the group consisting of a polyol and a surfactant.

118. The formulation of claim 117, further comprising a stabilizer.

119. The formulation of claim 110, further comprising mannitol, polysorbate 80 and methionine.

120. The formulation of claim 110, wherein the buffered solution further comprises phosphate or citrate.

121. The formulation of claim 110, wherein the pH is about 5 or less.

122. The formulation of claim 110, wherein the antibody is a human antibody, or antigen binding portion thereof, capable of binding to an epitope of a p40 subunit of IL-12/IL-23.

123. The formulation of claim 122, wherein the human antibody, or antigen binding portion thereof, binds to an epitope of the p40 subunit of IL-12/IL-23 to which an antibody selected from the group consisting of Y61 and J695 binds.

124. The formulation of claim 123, wherein the human antibody is the antibody J695, or an antigen binding portion thereof.

125. The formulation of any one of claims 110-124, which has a shelf life of at least 24 months.

126. The formulation of any one of claims 110-124, which maintains stability following at least 5 freeze/thaw cycles of the formulation.

127. An aqueous pharmaceutical formulation comprising
(a) 1-250 mg/ml of a human antibody that binds to an epitope of a p40 subunit of IL-12/IL-23,
(b) 1-10% mannitol,
(c) 0.001%-0.1% polysorbate-80,
(d) 1-50 mM methionine, and
(e) 1-100 mM histidine, with a pH of 5 to 7,
wherein the formulation is substantially free of metal.

128. The formulation of claim 127, wherein the metal is present at a concentration selected from the group consisting of less than about 5,060 ppb, less than about 1,060 ppb, less than about 560 ppb, less than about 310 ppb, less than about 160 ppb, less than about 110 ppb and less than about 70 ppb.

129. The formulation of claim 128, wherein the metal is present at a concentration of less than about 160 ppb.

130. The formulation of claim 128, wherein the metal is present at a concentration of less than about 70 ppb.

131. The formulation of claim 127, wherein the human antibody, or antigen binding portion thereof, binds to an epitope of the p40 subunit of IL-12/IL-23 to which an antibody selected from the group consisting of Y61 and J695 binds.

132. The formulation of claim 131, wherein the human antibody is the antibody J695, or an antigen binding portion thereof.

133. The formulation of any one of claims 127-132, which has a shelf life of at least 24 months.

134. The formulation of any one of claims 127-132, which maintains stability following at least 5 freeze/thaw cycles of the formulation.

135. An aqueous pharmaceutical formulation comprising
(a) about 100 mg/ml of a human antibody that binds to an epitope of a p40 subunit of IL-12/IL-23,
(b) about 4% mannitol,
(b) about 0.01% polysorbate-80,
(c) about 10 mM methionine, and
(d) about 10 mM histidine, with a pH of about 6.

136. The formulation of claim 135, wherein the human antibody, or antigen binding portion thereof, binds to an epitope of the p40 subunit of IL-12/IL-23 to which an antibody selected from the group consisting of Y61 and J695 binds.

137. The formulation of claim 136, wherein the human antibody is the antibody J695, or an antigen binding portion thereof.

138. The formulation of any one of claims 135-137, which is substantially free of metal.

139. The formulation of any one of claims 135-137, further comprising a metal chelator.

140. The formulation of claim 135, wherein the metal is present at a concentration selected from the group consisting of less than about 5,060 ppb, less than about 1,060 ppb, less than about 560 ppb, less than about 310 ppb, less than about 160 ppb, less than about 110 ppb and less than about 70 ppb.

141. The formulation of claim 140, wherein the metal is present at a concentration of less than about 160 ppb.

142. The formulation of claim 140, wherein the metal is present at a concentration of less than about 70 ppb.

143. The formulation of any one of claims 135-137, which has a shelf life of at least 24 months.

144. The formulation of any one of claims 135-137, which maintains stability following at least 5 freeze/thaw cycles of the formulation.
